(19) United States
(12) Patent Application Publication
(10) Pub. No.: US 2011/0230471 A1
(43) Pub. Date: Sep. 22, 2011

(54) BENZONAPHTYRIDINE COMPOUNDS AS INHIBITORS OF AUTOTAXIN

(75) Inventors: Wolfgang Staehle, Ingelheim (DE); Ingo Kober, Gross-Gerau (DE); Kai Schiemann, Seeheim-Jugenheim (DE); Melanie Schultz, Darmstadt (DE); Dirk Wienke, Darmstadt (DE)

(73) Assignee: MERCK PATENT GESELLSCHAFT MIT BESCHRANKTER HAFTUNG, DARMSTADT (DE)

(21) Appl. No.: 13/131,696

(22) PCT Filed: Nov. 5, 2009
(86) PCT No.: PCT/EP2009/007930

§ 371 Date: May 27, 2011

(30) Foreign Application Priority Data
Nov. 28, 2008 (DE) 10 2008 059 578.0

(51) Int. Cl. A61K 31/538; C07D 471/04; A61K 31/496; A61K 31/551; A61K 31/5377; A61K 31/4745; A61P 35/00

(52) U.S. Cl. 514/218; 544/361; 514/253.03; 540/575; 544/121; 514/230.8; 514/232.8; 544/105; 514/230.5; 544/126; 546/81; 514/292

(57) ABSTRACT

Compounds of the formula (I), in which R¹, R², R³, R⁴, R⁵, D, Z, X, Y, m and p have the meanings indicated in Claim 1, can and be employed for the treatment of tumours.

### BACKGROUND OF THE INVENTION

The invention was based on the object of finding novel compounds having valuable properties, in particular those which can be used for the preparation of medicaments.

The present invention relates to compounds and to the use of compounds for the treatment of diseases which are accompanied by an increase in the lysophosphatidic acid level, furthermore to pharmaceutical compositions which comprise these compounds.

In detail, the present invention relates to compounds of the formula I, which preferably inhibit one or more enzymes which regulate and/or modulate the lysophosphatidic acid (or LPA for short) level, to compositions which comprise these compounds, and to processes for the use thereof for the treatment of diseases and complaints, such as angiogenesis, cancer, tumour formation, growth and propagation, arteriosclerosis, ocular diseases, choroidal neovascularisation and diabetic retinopathy, inflammatory diseases, arthritis, neurodegeneration, restenosis, wound healing or transplant rejection. In particular, the compounds according to the invention are suitable for the therapy or prophylaxis of cancer diseases.

Autotaxin (ATX) is an enzyme which is responsible for the increase in the lysophosphatidic acid level in ascites and plasma (Xu et al. 1995, Clinical Cancer Research Vol. 1, page 1223 and Xu et al. 1995, Biochem. J. Vol-309, page 933). ATX converts lysophatidylcholine (LPC) into lysophosphatidic acid (Tokumura et al. 2002, J. Biol. Chem., Vol 277, page 39436 and Umezu-Gozo et al. 2002, J. Biol. Chem., Vol. 158, page 227) LPA is an intercellular lipid mediator which influences a multiplicity of biological and biochemical processes, such as, for example, smooth muscle contraction, thrombocyte aggregation and apoptosis (Tigyi et al. 2003 Prog. Lipid Res. Vol 42, page. 498 and Mills et al. 2003 Nat. Rev. Cancer Vol. 3, page 582 and Lynch et al. 2001 Prost. Lipid Med. Vol. 64, page 33). In addition, LPA can be found in increased concentrations in plasma and ascites fluid from ovarian cancer patients in the early and late phase. LPA plays a role there in tumour cell proliferation and invasion thereof into neighbouring tissue, which can result in metastasisation (Xu et al. 1995, Clinical Cancer Research Vol. 1, page 1223 and Xu et al. 1995, Biochem. J. Vol-309, page 933). These biological and phatobiological processes are switched on by the activation by LPA of G-protein-coupled receptors (Contos et al. 2000, Mol. Pharm. Vol 58, page. 1188).

For this reason, it is desirable to lower the LPA level for the treatment of tumour patients. This can be achieved by the inhibition of enzymes which are involved in LPA biosynthesis, such as, for example, autotaxin (ATX, Sano et al. 2002, J. Biol. Chem. Vol. 277, page 21197 and Aoki et al. 2003, J. Biol. Chem. Vol. 277 page 48737). Autotaxin belongs to the enzyme family of the nucleotides pyrophosphatases and phosphodiesterases (Goding et al. 1998, Immunol. Rev. Vol. 161, page 11) and represents an important starting point in antitumour therapy (Mills et al. 2003 Nat. Rev. Cancer Vol. 3, page 582 and Goto et al. 2004 J. Cell. Biochem. Vol. 92, page 1115) since it is expressed to an increased extent in tumours and causes tumour cell proliferation and invasion into neighbouring tissue, which can result in metastases formation (Nam et al. 2000, Oncogene, Vol. 19 page 241). In addition, autotaxin together with other angiogenetic factors causes blood vessel formation in the course of angiogenesis (Nam et al. 2001, Cancer Res. Vol. 61 page. 6938). Angiogenesis is an important process in tumour growth, which ensures supply of the tumour with nutrients.

For this reason, inhibition of angiogenesis is an important starting point in cancer and tumour therapy, with which the tumour can be starved to a certain extent (Folkman, 2007, Nature Reviews Drug Discovery Vol. 6, page 273-286).

Surprisingly, it has been found that the compounds according to the invention cause specific inhibition of the enzyme family of the nucleotides pyrophosphatases and phosphodiesterases, in particular autotaxin. The compounds according to the invention preferably exhibit an advantageous biological activity, which can easily be detected in the test described, for example, herein. In tests of this type, the compounds according to the invention preferably exhibit and cause an inhibiting effect, which is usually documented by 1050 values in a suitable range, preferably in the micromolar range and more preferably in the nanomolar range.

In general, all solid and non-solid tumours can be treated with the compounds of the formula I, such as, for example, monocytic leukaemia, brain, urogenital, lymphatic system, stomach, laryngeal, ovarian and lung carcinoma, including lung adenocarcinoma and small-cell lung carcinoma. Further examples include prostate, pancreatic and breast carcinoma.

As discussed herein, effects of the compound according to the invention are relevant for various diseases. Accordingly, the compounds according to the invention are useful in the prophylaxis and/or treatment of diseases which are influenced by inhibition of one or more nucleotides pyrophosphatases and/or phosphodiesterases, in particular autotaxin.

The present invention therefore relates to compounds according to the invention as medicaments and/or medicament active ingredients in the treatment and/or prophylaxis of the said diseases and to the use of compounds according to the invention for the preparation of a pharmaceutical agent for the treatment and/or prophylaxis of the said diseases, and also to a method for the treatment of the said diseases comprising the administration of one or more compounds according to the invention to a patient in need of such administration.

It can be shown that the compounds according to the invention have an advantageous action in a xenotransplant tumour model.

The host or patient can belong to any mammalian species, for example a primate species, in particular humans; rodents, including mice, rats and hamsters; rabbits; horses, cattle, dogs, cats, etc. Animal models are of interest for experimental investigations, where they provide a model for the treatment of a human disease.

The sensitivity of a certain cell to treatment with the compounds according to the invention can be determined by testing in vitro. Typically, a culture of the cell is combined with a compound according to the invention at various concentrations for a time which is sufficient to enable the active agents to induce cell death or to inhibit cell migration or to block the cellular secretion of angiogenesis-promoting substances, usually between approximately one hour and one week. For testing in vitro, cultivated cells from a biopsy sample can be used. The viable cells remaining after the treatment are then counted.

The dose varies depending on the specific compound used, the specific disease, the patient status, etc. Typically, a therapeutic dose is sufficient to considerably reduce the undesired cell population in the target tissue, while the viability of the patient is maintained. The treatment is generally continued until a considerable reduction has occurred, for example at least about a 50% reduction in the cell burden, and can be continued until essentially no undesired cells can be detected in the body.

### PRIOR ART

Compounds which are capable of inhibiting autotaxin are described in Peng et al. Bioorganic & Medicinal Chemistry Letters (17, 2007, page 1634-1640). The compounds described therein are lipid analogues, which do not have any structural features in common with the compounds according to the invention.

Other naphthyridine derivatives are described in EP 0 997 462.

### SUMMARY OF THE INVENTION

The invention relates to compounds of the formula I

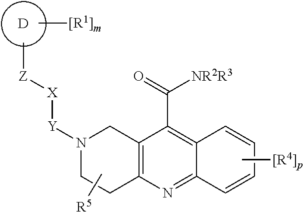

in which
D denotes Ar or Het¹,
Het¹ denotes a mono- or bicyclic, saturated, unsaturated or aromatic heterocycle having 1 to 4 N, O and/or S atoms, which is unsubstituted or may be mono-, di or trisubstituted by Hal, A, OA, Ar, OH and or ═O,
R¹ in each case, independently of one another, denotes H, Hal, OA, OH, A, phenyl, Het² or CN mono- or polysubstituted,
Het² denotes a monocyclic, saturated heterocycle having 1-3N and/or O atoms, which is unsubstituted or may be mono- or disubstituted by ═O,
R⁴ in each case, independently of one another, denotes H, Hal, OA, OH, A, mono- or polysubstituted,
X, Y each, independently of one another, are absent, or denote —CH₂—, —(CH₂)₂—, —CO— or —CHOH—, where only one of the radicals X or Y may be absent,
R², R³ each, independently of one another, denote R; R² and R³ together also denote an alkylene chain having 2-6C atoms, in which, in addition, one CH₂ group replaced by O, NH or NA',
A′ denotes alkyl having 1-6C atoms, or CH₂CH₂OH, COO(CH₂)ₙAr, (CH₂)ₙAr, (CH₂)ₙHet², (CH₂)ₙNA₂ or Cyc,
R⁵ denotes H, Hal, NH₂, OH, OA or A,
R denotes H, A, Cyc, (CH₂)ₙAr or (CH₂)ₙHet mono- or polysubstituted,
Z denotes O, NH, —CH(CONHA)NH—, CH₂NHCONH, —CH═CH— or is absent,
Cyc denotes cyclic alkyl having 3-7C atoms,
A denotes linear or branched alkyl having 1-10C atoms, in which 1-7 H atoms may be replaced by OR, CN, NR₂, F and/or Cl and/or in which one or two non-adjacent CH₂ groups may be replaced by O, NH, S, SO, SO₂ and/or by CH═CH groups, or cyclic alkyl having 3-7C atoms,
Ar denotes phenyl, indanyl, naphthyl or biphenyl, each of which is unsubstituted or mono-, di-, tri-, tetra- or pentasubstituted by Hal, A, (CR₂)ₙOR, O(CR₂)ₙAr², (CR₂)ₙNR₂, SR, NO₂, CN, COOR, CONR₂, NRCOA, NRSO₂A, SO₂NR₂, S(O)ₘA, CO-Het, (CR₂)ₙHet, O(CR₂)ₙNR₂, O(CR₂)ₙHet, NHCOOA, NHCONR₂, NHCOO(CR₂)ₙNR₂, NHCOO(CR₂)ₙHet, CR═CRAr², SO₂Het, NHCONH(CR₂)ₙNR₂, NHCONH(CR₂)ₙHet, OCONH(CR₂)ₙNR₂, CONH(CR₂)ₙHet, CONR(CR₂)ₙNR₂, CONR(CR₂)ₙHet and/or COA,
Het denotes a mono-, bi- or tricyclic saturated, unsaturated or aromatic heterocycle having 1 to 4 N, O and/or S atoms, which may be unsubstituted or mono-, di- or trisubstituted by Hal, A, Ar², O(CR₂)ₙAr², (CR₂)ₙOR, (CR₂)ₙNR₂, SR, NO₂, CN, COOR, CONR₂, NRCOA, NRSO₂A, SO₂NR₂, S(O)ₙA, CO-Het², (CR₂)ₙHet², O(CR₂)ₙNR₂, O(CR₂)ₙHet², NHCOOA, NHCONR₂, NHCOO(CR₂)ₙNR₂, NHCOO(CR₂)ₙHet², NHCONH(CR₂)ₙNR₂, NHCONH(CR₂)ₙHet², OCONH(CR₂)ₙNR₂, OCONH(CR₂)ₙHet², CO—Het², CHO, COA, ═S, ═NH, ═NA and/or ═O (carbonyl oxygen),
Hal denotes F, Cl, Br or I,
n denotes 0, 1 or 2,
m denotes 0, 1, 2, 3, 4, or,
p denotes 1, 2, 3, or 4,
   and pharmaceutically usable salts and stereoisomers thereof, including mixtures thereof in all ratios.

Compounds of the formula I also mean pharmaceutically usable derivatives thereof, optically active forms (stereoisomers), tautomers, polymorphs, enantiomers, racemates, diastereomers and the hydrates and solvates of these compounds. The term solvates of the compounds is taken to mean adductions of inert solvent molecules onto the compounds which form owing to their mutual attractive force. solvates are, for example, mono- or dihydrates or alcoholates.

Pharmaceutically usable derivatives are taken to mean, for example, the salts of the compounds according to the invention and also so-called prodrug compounds.

Prodrug derivatives are taken to mean compounds of the formula I which have been modified by means of, for example, alkyl or acyl groups, sugars or oligopeptides and which are rapidly cleaved in the organism to form the effective compounds according to the invention.

These also include biodegradable polymer derivatives of the compounds according to the invention, as described, for example, in Int. J. Pharm. 115, 61-67 (1995).

The expression “effective amount” denotes the amount of a medicament or of a pharmaceutical active ingredient which causes in a tissue, system, animal or human biological or medical response which is sought or desired, for example, by a researcher or physician.

In addition, the expression “therapeutically effective amount” denotes an amount which, compared with a corresponding subject who has not received this amount, has the following consequence:

improved treatment, healing, prevention or elimination of a disease, syndrome, condition, complaint, disorder or side effects or also the reduction in the advance of a disease, complaint or disorder.

The expression “therapeutically effective amount” also encompasses the amounts which are effective for increasing normal physiological function.

The invention also relates to the use of mixtures of the compounds of the formula I, for example mixtures of two diastereomers, for example in the ratio 1:1, 1:2, 1:3, 1:4, 1:5, 1:10, 1:100 or 1:1000.

These are particularly preferably mixtures of stereoisomeric compounds.

The invention relates to the compounds of the formula I and salts thereof and to a process for the preparation of compounds of the formula I according to the patent claims and pharmaceutically usable salts, and stereoisomers thereof, characterised in that
for the preparation of compounds of the formula I,
a compound of the formula II

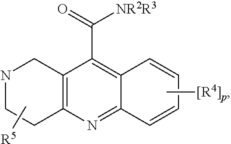

in which R², R³, R⁴, R⁵ and p, have the meanings indicated in claim 1,
   is reacted with a compound of the formula III or IV

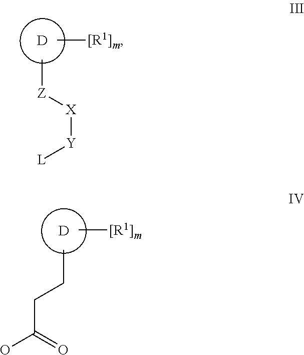

in which
R¹, m, D, Z, X and Y have the meanings indicated in claim 1 and L is a halogen, tosylate, mesylate or triflate,
   and/or a base or acid of the formula I is converted into one of its salts.

A denotes alkyl and is preferably unbranched (linear) or branched, and has 1, 2, 3, 4, 5, 6, 7, 8, 9 or 10C atoms. Alkyl preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, further preferably, for example, trifluoromethyl.

Alkyl very particularly preferably denotes alkyl having 1, 2, 3, 4, 5 or 6C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. Alkyl also denotes cycloalkyl.

Cycloalkyl preferably denotes cyclopropyl, cyclobutyl, cylopentyl, cyclohexyl or cycloheptyl.

Alk preferably denotes unbranched or branched alkylene having 1, 2, 3 or 4 C atoms, particularly preferably methylene, ethylene, propylene or butylene.

Ar′ preferably denotes phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl, o-, m- or p-bromo-phenyl, o-, m- or p-chlorophenyl, o-, m- or p-(methylsulfonamido)phenyl, o-, m- or p-(methylsulfonyl)phenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodo-phenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl, 2,5-dimethyl-4-chlorophenyl, naphthyl or biphenyl.

Ar preferably denotes phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl, 2,5-dimethyl-4-chlorophenyl, naphthyl or biphenyl.

Ar furthermore preferably denotes phenyl, naphthyl or biphenyl substituted phenyl, indanyl, naphthyl or biphenyl, each of which is unsubstituted or mono-, di-, tri-, tetra- or pentasubstituted by Hal, A and/or (CR₂)ₙOR.

Ar² preferably denotes phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-methoxyphenyl, o-, m- or p-ethoxyphenyl o-, m- or p-fluorophenyl, o-, m- or p-bromophenyl, o-, m- or p-chlorophenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,5- or 3,4-dimethoxyphenyl, 2,3,4-, 2,3,5-, 2,3,6-, 2,4,6- or 3,4,5-trichlorophenyl, 2,4,6-trimethoxyphenyl, 2-hydroxy-3,5-dichlorphenyl, p-iodophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-fluoro-4-methoxyphenyl, 2,5-dimethyl-4-chlorophenyl.

Irrespective of further substitutions, Het¹ denotes, for example, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-iso-indolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-5-yl, 2,1,3-benzoxadiazol-5-yl or dibenzofuranyl. The heterocyclic radicals may also be partially or fully hydrogenated.

Irrespective of further substitutions, Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, further preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-dihydrobenzoxazolyl, 2,3-dihydrobenzimidazolyl, 1,3-dihydroindole, 2-oxo-1,3-dihydroindole or 2-oxo-2,3-dihydrobenzimidazolyl. Het¹ furthermore preferably denotes a monocyclic aromatic heterocycle having 1 to 4 N, O and/or S atoms.

Het¹ particularly preferably denotes piperazyl, morpholinyl, piperidinyl, pyrrolidinyl, furanyl, thienyl, pyrrolyl, imidazolyl, pyrazolyl, oxazolyl, isoxazolyl, thiazolyl, isothiazolyl, pyridyl, pyrimidinyl, triazolyl, benzotriazylyl, benzofuranyl, 2,3-dihydrobenzoxazolyl, benzoxazolyl, dihydrobenzofuranyl or tetrazolyl, each of which is unsubstituted or mono-, di- or trisubstituted by A and/or (CH₂)ₙAᵣ.

Het¹ furthermore denotes a saturated or aromatic heterocycle, which may be substituted by piperazine, morpholine, piperidine and pyrrolidine.

Irrespective of further substitutions, Het denotes, for example, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-5-yl, 2,1,3-benzoxadiazol-5-yl or dibenzofuranyl. The heterocyclic radicals may also be partially or fully hydrogenated. Irrespective of further substitutions, Het can thus also denote, for example, 2,3-dihydro-2-, -3-, -4- or -5-furyl, 2,5-dihydro-2-, -3-, -4- or 5-furyl, tetra-hydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, further preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-dihydrobenzoxazolyl, 2,3-dihydrobenzimidazolyl, 1,3-dihydroindole, 2-oxo-1,3-dihydroindole or 2-oxo-2,3-dihydrobenzimidazolyl.

Het furthermore preferably denotes a monocyclic saturated, unsaturated or aromatic heterocycle having 1 to 4 N, O and/or S atoms, which may be unsubstituted or mono-, di- or trisubstituted by A, Ar², (CR₂)ₙHet² and/or (CR₂)ₙOR.

Het very particularly preferably denotes piperidinyl, piperazinyl, pyrrolidinyl, morpholinyl, furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl or pyrazinyl, each of which is unsubstituted or mono- or disubstituted by A, Ar², (CR₂)ₙHet² and/or (CR₂)ₙOR.

Hal preferably denotes F, Cl or Br, but also I, particularly preferably Br or Cl.

The indices have the following preferred meanings

m 1 or 2,
n 0, 1, 2, 3, 4 or 5,
p 1, 2, 3 or 4.

Throughout the invention, all radicals which occur more than once, such as, for example, R, may be identical or different, i.e. are independent of one another.

The compounds of the formula I may have one or more chiral centres and can therefore occur in various stereoisomeric forms. The formula I encompasses all these forms.

Accordingly, the invention relates, in particular, to the compounds of the formula I in which at least one of the said radicals has one of the preferred meanings indicated above.

Some preferred groups of compounds may be expressed by the following sub-formulae Ia to Ie, which conform to the formula I and in which the radicals not designated in greater detail have the meaning indicated for the formula I, but in which
in Ia R¹ denotes H, Hal, CN, phenyl, OA or OH;
in Ib R⁴ denotes H, Hal, A or OH;
in Ic R⁵H;
in Id R², R³ together denotes morpholinyl, piperazinyl, 1-methylpiperazinyl, 1-ethyl-4-methylpiperazinyl, 2-(4-methylpiperazin-1-yl)-ethyl, 1-methyl-4-propylpiperazinyl, 1-cyclopentyl-4-methylpiperazinyl, 1-benzyl-4-methyl-1,4-diazepanyl or 1-benzyl-4-methylpiperazinyl;
in Ie Het¹ particularly preferably denotes piperazyl, morpholinyl, pperidinyl, pyrrolidinyl, furanyl, thienyl, pyrrolyl, imidazolyl, pyrazolyl, oxazolyl, isoxazolyl, thiazolyl, isothiazolyl, pyridyl, pyrimidinyl, triazolyl, benzotriazylyl, benzofuranyl, 2,3-dihydrobenzoxazolyl, benzoxazolyl, dihydrobenzofuranyl or tetrazolyl, each of which is unsubstituted or mono-, di- or trisubstituted by A and/or (CH₂)ₙAr.
In If Het² particularly preferably denotes pyrrolidinyl, morpholinyl, piperidinyl or piperazinyl, each of which is unsubstituted or mono- or disubstituted by Hal, OH, OA, A and/or ═O.
in Ig R¹ denotes H, Hal, CN, phenyl, OA or OH;
   R⁴ denotes H, Hal, A or OH;
   R⁵ denotes H and
   R², R³ together denote morpholinyl, piperazinyl, 1-methylpiperazinyl, 1-ethyl-4-methylpiperazinyl, 2-(4-methylpiperazin-1-yl)-ethyl, 1-methyl-4-propylpiperazinyl, 1-cyclopentyl-4-methylpiperazinyl, 1-benzyl-4-methyl-1,4-diazepanyl or 1-benzyl-4-methylpiperazinyl,
   Het¹ particularly preferably denotes piperazyl, morpholinyl, pperidinyl, pyrrolidinyl, furanyl, thienyl, pyrrolyl, imidazolyl, pyrazolyl, oxazolyl, isoxazolyl, thiazolyl, isothiazolyl, pyridyl, pyrimidinyl, triazolyl, benzotriazylyl, benzofuranyl, 2,3-dihydrobenzoxazolyl, benzoxazolyl, dihydrobenzofuranyl or tetrazolyl, each of which is unsubstituted or mono-, di- or trisubstituted by A and/or (CH₂)ₙAr,
   Het² particularly preferably denotes pyrrolidinyl, morpholinyl, piperidinyl or piperazinyl, each of which is unsubstituted or mono- or di substituted by Hal, OH, OA, A and/or ═O,
      and pharmaceutically usable salts and stereoisomers thereof, including mixtures thereof in all ratios.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants known per se which are not mentioned here in greater detail.

The starting materials can, if desired, also be formed in situ by not isolating them from the reaction mixture, but instead immediately converting them further into the compounds of the formula I.

Compounds of the formula I can preferably be obtained by reacting a compound of the formula II with a compound of the formula III.

Depending on the conditions used, the reaction time is between a few minutes and 14 days, the reaction temperature is between about −30° and 140°, normally between −10° and 90°, in particular between about 0° and about 70°.

Examples of suitable inert solvents are hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Particular preference is given to pyridine, acetonitrile, dichloromethane and/or DMF.

The starting compounds of the formulae II, II and IV are generally known. If they are novel, however, they can be prepared by methods known per se. The starting materials are generally also commercially available.

The said compounds according to the invention can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds of the formula I are for the most part prepared by conventional methods. If the compound of the formula I contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline-earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds of the formula I are likewise included. In the case of certain compounds of the formula I, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds of the formula I include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.

Furthermore, the base salts of the compounds according to the invention include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline-earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine(benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)-methylamine (tromethamine), but this is not intended to represent a restriction.

Compounds of the present invention which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds of the formula I are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the formula I are formed with metals or amines, such as alkali metals and alkaline-earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the expression “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The invention furthermore relates to medicaments comprising at least one compound of the formula I and/or pharmaceutically usable salts and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or drypressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a prespecified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula I and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, solvates and physiologically functional derivatives thereof can also be delivered using monoclonal anti-bodies as individual carriers to which the compound molecules are coupled.

The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I depends on a number of factors, including, for example, the age and weight of the animal, the precise condition that requires treatment, and its severity, the nature of the formulation and the method of administration, and is ultimately determined by the treating doctor or vet. However, an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or more usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The invention furthermore relates to medicaments comprising at least one compound of the formula I and/or pharmaceutically usable derivatives, solvates and stereoisomers thereof, including mixtures thereof in all ratios, and at least one further medicament active ingredient.

The invention also relates to a set (kit) consisting of separate packs of
(a) an effective amount of a compound of the formula I and/or pharmaceutically usable derivatives, solvates and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules, each containing an effective amount of a compound of the formula I and/or pharmaceutically usable derivatives, solvates and stereoisomers thereof, including mixtures thereof in all ratios, and an effective amount of a further medicament active ingredient in dissolved or lyophilised form.

The medicaments from Table 1 are preferably, but not exclusively, combined with the compounds of the formula I. A combination of the formula I and medicaments from Table I can also be combined with compounds of the formula VI.

TABLE 1

| | | |
|---|---|---|
| Alkylating agents | Cyclophosphamide | Lomustine |
| | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum agents | Cisplatin | Carboplatin |
| | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 |
| | Iproplatin | (Hoffrnann-La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-fluorouracil | Fludarabine |
| | Floxuridine | Pentostatin |
| | 2-chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-fluorodesoxycytidine | Irofulven (MGI Pharrna) |
| | Methotrexate | DMDC (Hoffmann-La |
| | Idatrexate | Roche) |
| | | Ethynylcytidine (Taiho) |
| Topoisomerase | Amsacrine | Rubitecan (SuperGen) |
| inhibitors | Epirubicin | Exatecan mesylate (Daiichi) |
| | Etoposide | Quinamed (ChemGenex) |
| | Teniposide or | Gimatecan (Sigma-Tau) |
| | mitoxantrone | Diflomotecan (Beaufour- |
| | Irinotecan (CPT-11) | Ipsen) |
| | 7-Ethyl-10- | TAS-103 (Taiho) |
| | hydroxycamptothecin | Elsamitrucin (Spectrum) |
| | Topotecan | J-107088 (Merck & Co) |
| | Dexrazoxanet | BNP-1350 (BioNumerik) |
| | (TopoTarget) | CKD-602 (Chong Kun |
| | Pixantrone (Novuspharrna) | Dang) |
| | Rebeccamycin analogue | KW-2170 (Kyowa Hakko) |
| | (Exelixis) | |
| | BBR-3576 (Novuspharrna) | |
| Antitumour | Dactinomycin (Actinomycin | Amonafide |
| antibiotics | D) | Azonafide |
| | Doxorubicin (Adriamycin) | Anthrapyrazole |
| | Deoxyrubicin | Oxantrazole |
| | Valrubicin | Losoxantrone |
| | Daunorubicin | Bleomycin sulfate |
| | (Daunomycin) | (Blenoxan) |
| | Epirubicin | Bleomycinic acid |
| | Therarubicin | Bleomycin A |
| | Idarubicin | Bleomycin B |
| | Rubidazone | Mitomycin C |
| | Plicamycinp | MEN-10755 (Menarini) |
| | Porfiromycin | GPX-100 (Gem |
| | Cyanomorpholinodoxorubicin | Pharmaceuticals) |
| | Mitoxantrone (Novantrone) | |
| Antimitotic agents | Paclitaxel | SB 408075 |
| | Docetaxel | (GlaxoSmithKline) |
| | Colchicine | E7010 (Abbott) |
| | Vinblastine | PG-TXL (Cell |
| | Vincristine | Therapeutics) |
| | Vinorelbine | IDN 5109 (Bayer) |
| | Vindesine | A 105972 (Abbott) |
| | Dolastatin 10 (NCI) | A 204197 (Abbott) |
| | Rhizoxin (Fujisawa) | LU 223651 (BASF) |
| | Mivobulin (Warner- | D 24851 (ASTA Medica) |
| | Lambert) | ER-86526 (Eisai) |
| | Cemadotin (BASF) | Combretastatin A4 (BMS) |
| | RPR 109881A (Aventis) | Isohomohalichondrin-B |
| | TXD 258 (Aventis) | (PharmaMar) |
| | Epothilone B (Novartis) | ZD 6126 (AstraZeneca) |
| | T 900607 (Tularik) | PEG-Paclitaxel (Enzon) |
| | T 138067 (Tularik) | AZ10992 (Asahi) |
| | Cryptophycin 52 (Eli Lilly) | !DN-5109 (Indena) |
| | Vinflunine (Fabre) | AVLB (Prescient |
| | Auristatin PE (Teikoku | NeuroPharma) |
| | hormone) | Azaepothilon B (BMS) |
| | BMS 247550 (BMS) | BNP-7787 (BioNumerik) |
| | BMS 184476 (BMS) | CA-4-prodrug (OXiGENE) |
| | BMS 188797 (BMS) | Dolastatin-10 (NrH) |
| | Taxoprexin (Protarga) | CA-4 (OXiGENE) |
| Aromatase | Aminoglutethimide | Exemestan |
| inhibitors | Letrozole | Atamestan (BioMedicines) |
| | Anastrazole | YM-511 (Yamanouchi) |
| | Formestan | |
| Thymidylate synthase | Pemetrexed (Eli Lilly) | Nolatrexed (Eximias) |
| inhibitors | ZD-9331 (BTG) | CoFactor ™ (BioKeys) |
| DNA antagonists | Trabectedin (PharmaMar) | Mafosfamide (Baxter |
| | Glufosfamide (Baxter | International) |
| | International) | Apaziquone (Spectrum |
| | Albumin + 32P (Isotope | Pharmaceuticals) |
| | Solutions) | O6-benzylguanine |
| | Thymectacin (NewBiotics) | (Paligent) |
| | Edotreotid (Novartis) | |
| Farnesyl | Arglabin (NuOncology | Tipifarnib (Johnson & |
| transferase | Labs) | Johnson) |
| inhibitors | Ionafarnib (Schering- | Perrillyl alcohol (DOR |
| | Plough) | BioPharma) |
| | BAY-43-9006 (Bayer) | |
| Pump inhibitors | CBT-1 (CBA Pharma) | Zosuquidar |
| | Tariquidar (Xenova) | trihydrochloride (Eli Lilly) |
| | MS-209 (Schering AG) | Biricodar dicitrate (Vertex) |
| Histone acetyl | Tacedinaline (Pfizer) | Pivaloyloxymethyl butyrate |
| transferase inhibitors | SAHA (Aton Pharma) | (Titan) |
| | MS-275 (Schering AG) | Depsipeptide (Fujisawa) |
| Metalloproteinase | Neovastat (Aeterna Laboratories) | CMT-3 (CollaGenex) |
| inhibitors | Marimastat (British Biotech) | BMS-275291 (Celltech) |
| Ribonucleoside | Gallium maltolate (Titan) | Tezacitabine (Aventis) |
| reductase inhibitors | Triapin (Vion) | Didox (Molecules for |
| | | Health) |
| TNF-alpha | Virulizin (Lorus Therapeutics) | Revimid (Celgene) |
| agonists/ | CDC-394 (Celgene) | |
| antagonists | | |
| Endothelin-A receptor | Atrasentan (Abbot) | YM-598 (Yamanouchi) |
| antagonists | ZD-4054 (AstraZeneca) | |
| Retinoic acid receptor | Fenretinide (Johnson & | Alitretinoin (Ligand) |
| agonists | Johnson) | |
| | LGD-1550 (Ligand) | |
| Immunomodulators | Interferon | Dexosome therapy (Anosys) |
| | Oncophage (Antigenics) | Pentrix (Australian Cancer |
| | GMK (Progenics) | Technology) |
| | Adenocarcinoma vaccine | JSF-154 (Tragen) |
| | (Biomira) | Cancer vaccine (Intercell) |
| | CTP-37 (AVI BioPharma) | Norelin (Biostar) |
| | JRX-2 (Immuno-Rx) | BLP-25 (Biomira) |
| | PEP-005 (Peplin Biotech) | MGV (Progenics) |
| | Synchrovax vaccines (CTL | !3-Alethin (Dovetail) |
| | Immuno) | CLL-Thera (Vasogen) |
| | Melanoma vaccine (CTL | |
| | Immuno) | |
| | p21-RAS vaccine (Gem- | |
| | Vax) | |
| Hormonal and | Oestrogens | Prednisone |
| antihormonal | Conjugated oestrogens | Methylprednisolone |
| agents | Ethynyloestradiol | Prednisolone |
| | chlorotrianisene | Aminoglutethimide |
| | Idenestrol | Leuprolide |
| | Hydroxyprogesterone | Goserelin |
| | caproate | Leuporelin |
| | Medroxyprogesterone | Bicalutamide |
| | Testosterone | Flutamide |
| | Testosterone propionate | Octreotide |
| | Fluoxymesterone | Nilutamide |
| | Methyltestosterone | Mitotan |
| | Diethylstilbestrol | P-04 (Novogen) |
| | Megestrol | 2-Methoxyoestradiol (EntreMed) |
| | Tamoxifen | Arzoxifen (Eli Lilly) |
| | Toremofin | |
| | Dexamethasone | |
| Photodynamic | Talaporfin (Light Sciences) | Pd-bacteriopheophorbide |
| agents | Theralux (Theratechnologies) | (Yeda) |
| | Motexafin gadolinium | Lutetium texaphyrin |
| | (Pharmacyclics) | (Pharmacyclics) |
| | | Hypericin |
| Tyrosine kinase | Imatinib (Novartis) | Kahalide F (PharmaMar) |
| inhibitors | Leflunomide (Sugen/ | CEP-701 (Cephalon) |
| | Pharmacia) | CEP-751 (Cephalon) |
| | ZDI839 (AstraZeneca) | MLN518 (Millenium) |
| | Erlotinib (Oncogene | PKC412 (Novartis) |
| | Science) | Phenoxodiol O |
| | Canertjnib (Pfizer) | Trastuzumab (Genentech) |
| | Squalamine (Genaera) | C225 (ImClone) |
| | SU5416 (Pharmacia) | rhu-Mab (Genentech) |
| | SU6668 (Pharmacia) | MDX-H210 (Medarex) |
| | ZD4190 (AstraZeneca) | 2C4 (Genentech) |
| | ZD6474 (AstraZeneca) | MDX-447 (Medarex) |
| | Vatalanib (Novartis) | ABX-EGF (Abgenix) |
| | PKI166 (Novartis) | IMC-1C11 (ImClone) |
| | GW2016 (GlaxoSmith- | |
| | Kline) | |
| | EKB-509 (Wyeth) | |
| | EKB-569 (Wyeth) | |
| Various agents | SR-27897 (CCK-A | BCX-1777 (PNP inhibitor, |
| | inhibitor, Sanofi- | BioCryst) |
| | Synthelabo) | Ranpirnase (ribonuclease |
| | Tocladesine (cyclic AMP | stimulant, Alfacell) |
| | agonist, Ribapharm) | Galarubicin (RNA synthesis |
| | Alvocidib (CDK inhibitor, | inhibitor, Dong-A) |
| | Aventis) | Tirapazamine (reducing |
| | CV-247 (COX-2 inhibitor, | agent, SRI International) |
| | Ivy Medical) | N-Acetylcysteine (reducing |
| | P54 (COX-2 inhibitor, | agent, Zambon) |
| | Phytopharm) | R-Flurbiprofen (NF-kappaB |
| | CapCell ™ (CYP450 | inhibitor, Encore) |
| | stimulant, Bavarian Nordic) | 3CPA (NF-kappaB |
| | GCS-IOO (gal3 antagonist, | inhibitor, Active Biotech) |
| | GlycoGenesys) | Seocalcitol (vitamin D |
| | G17DT immunogen | receptor agonist, Leo) |
| | (gastrin inhibitor, Aphton) | 131-I-TM-601 (DNA |
| | Efaproxiral (oxygenator, | antagonist, |
| | Allos Therapeutics) | TransMolecular) |
| | PI-88 (heparanase | Eflornithin (ODC inhibitor, |
| | inhibitor, Progen) | ILEX Oncology) |
| | Tesmilifen (histamine antagonist, | Minodronic acid |
| | YM BioSciences) | (osteoclast inhibitor, |
| | Histamine (histamine H2 | Yamanouchi) |
| | receptor agonist, Maxim) | Indisulam (p53 stimulant, |
| | Tiazofurin (IMPDH | Eisai) |
| | inhibitor, Ribapharm) | Aplidin (PPT inhibitor, |
| | Cilengitide (integrin | PharmaMar) |
| | antagonist, Merck KGaA) | Rituximab (CD20 antibody, |
| | SR-31747 (IL-1 antagonist, | Genentech) |
| | Sanofi-Synthelabo) | Gemtuzumab (CD33 |
| | CCI-779 (mTOR kinase | antibody, Wyeth Ayerst) |
| | inhibitor, Wyeth) | PG2 (haematopoiesis |
| | Exisulind (PDE-V inhibitor, | promoter, |
| | Cell Pathways) | Pharmagenesis) |
| | CP-461 (PDE-V inhibitor, | Immunol ™ (triclosan |
| | Cell Pathways) | mouthwash, Endo) |
| | AG-2037 (GART inhibitor, | Triacetyluridine (uridine |
| | Pfizer) | prodrug, Wellstat) |
| | WX-UK1 | SN-4071 (sarcoma agent, |
| | (plasminogen activator | Signature BioScience) |
| | inhibitor, Wilex) | TransMID-107 ™ |
| | PBI-1402 (PMN stimulant, | (immunotoxin, KS |
| | ProMetic LifeSciences) | Biomedix) |
| | Bortezomib (proteasome | PCK-3145 (apoptosis |
| | inhibitor, Millennium) | promoter, Procyon) |
| | SRL-172 (T-cell stimulant, | Doranidazole (apoptosis |
| | SR Pharma) | promoter, Pola) |
| | TLK-286 (glutathione-S | CHS-828 (cytotoxic agent, |
| | transferase inhibitor, | Leo) |
| | Telik) | trans-Retinoic acid |
| | PT-100 (growth factor | (differentiator, NIH) |
| | agonist, Point | MX6 (apoptosis promoter, |
| | Therapeutics) | MAXIA) |
| | Midostaurin (PKC inhibitor, | Apomine (apoptosis |
| | Novartis) | promoter, ILEX Oncology) |
| | Bryostatin-1 (PKC | Urocidin (apoptosis |
| | stimulant, GPC Biotech) | promoter, Bioniche) |
| | CDA-II (apoptosis | Ro-31-7453 (apoptosis |
| | promoter, Everlife) | promoter, La Roche) |
| | SDX-101 (apoptosis | Brostallicin (apoptosis |
| | promoter, Salmedix) | promoter, Pharmacia) |
| | Ceflatonin (apoptosis | |
| | promoter, ChemGenex) | |
| Alkylating agents | Cyclophosphamide | Lomustine |
| | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum agents | Cisplatin | Carboplatin |
| | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 |
| | Iproplatin | (Hoffrnann-La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-fluorouracil | Fludarabine |
| | Floxuridine | Pentostatin |
| | 2-chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-fluorodesoxycytidine | Irofulven (MGI Pharrna) |
| | Methotrexate | DMDC (Hoffmann-La |
| | Idatrexate | Roche) |
| | | Ethynylcytidine (Taiho) |
| Topoisomerase | Amsacrine | Rubitecan (SuperGen) |
| inhibitors | Epirubicin | Exatecan mesylate (Daiichi) |
| | Etoposide | Quinamed (ChemGenex) |
| | Teniposide or | Gimatecan (Sigma-Tau) |
| | mitoxantrone | Diflomotecan (Beaufour- |
| | Irinotecan (CPT-11) | Ipsen) |
| | 7-Ethyl-10- | TAS-103 (Taiho) |
| | hydroxycamptothecin | Elsamitrucin (Spectrum) |
| | Topotecan | J-107088 (Merck & Co) |
| | Dexrazoxanet | BNP-1350 (BioNumerik) |
| | (TopoTarget) | CKD-602 (Chong Kun |
| | Pixantrone (Novuspharrna) | Dang) |
| | Rebeccamycin analogue | KW-2170 (Kyowa Hakko) |
| | (Exelixis) | |
| | BBR-3576 (Novuspharrna) | |
| Antitumour | Dactinomycin (Actinomycin | Amonafide |
| antibiotics | D) | Azonafide |
| | Doxorubicin (Adriamycin) | Anthrapyrazole |
| | Deoxyrubicin | Oxantrazole |
| | Valrubicin | Losoxantrone |
| | Daunorubicin | Bleomycin sulfate |
| | (Daunomycin) | (Blenoxan) |
| | Epirubicin | Bleomycinic acid |
| | Therarubicin | Bleomycin A |
| | Idarubicin | Bleomycin B |
| | Rubidazone | Mitomycin C |
| | Plicamycinp | MEN-10755 (Menarini) |
| | Porfiromycin | GPX-100 (Gem |
| | Cyanomorpholinodoxorubicin | Pharmaceuticals) |
| | Mitoxantrone (Novantrone) | |
| Antimitotic agents | Paclitaxel | SB 408075 |
| | Docetaxel | (GlaxoSmithKline) |
| | Colchicine | E7010 (Abbott) |
| | Vinblastine | PG-TXL (Cell |
| | Vincristine | Therapeutics) |
| | Vinorelbine | IDN 5109 (Bayer) |
| | Vindesine | A 105972 (Abbott) |
| | Dolastatin 10 (NCI) | A 204197 (Abbott) |
| | Rhizoxin (Fujisawa) | LU 223651 (BASF) |
| | Mivobulin (Warner- | D 24851 (ASTA Medica) |
| | Lambert) | ER-86526 (Eisai) |
| | Cemadotin (BASF) | Combretastatin A4 (BMS) |
| | RPR 109881A (Aventis) | Isohomohalichondrin-B |
| | TXD 258 (Aventis) | (PharmaMar) |
| | Epothilone B (Novartis) | ZD 6126 (AstraZeneca) |
| | T 900607 (Tularik) | PEG-Paclitaxel (Enzon) |
| | T 138067 (Tularik) | AZ10992 (Asahi) |
| | Cryptophycin 52 (Eli Lilly) | !DN-5109 (Indena) |
| | Vinflunine (Fabre) | AVLB (Prescient |
| | Auristatin PE (Teikoku | NeuroPharma) |
| | hormone) | Azaepothilon B (BMS) |
| | BMS 247550 (BMS) | BNP-7787 (BioNumerik) |
| | BMS 184476 (BMS) | CA-4-prodrug (OXiGENE) |
| | BMS 188797 (BMS) | Dolastatin-10 (NrH) |
| | Taxoprexin (Protarga) | CA-4 (OXiGENE) |
| Aromatase | Aminoglutethimide | Exemestan |
| inhibitors | Letrozole | Atamestan (BioMedicines) |
| | Anastrazole | YM-511 (Yamanouchi) |
| | Formestan | |
| Thymidylate synthase | Pemetrexed (Eli Lilly) | Nolatrexed (Eximias) |
| inhibitors | ZD-9331 (BTG) | CoFactor ™ (BioKeys) |
| DNA antagonists | Trabectedin (PharmaMar) | Mafosfamide (Baxter |
| | Glufosfamide (Baxter | International) |
| | International) | Apaziquone (Spectrum |
| | Albumin + 32P (Isotope | Pharmaceuticals) |
| | Solutions) | O6-benzylguanine |
| | Thymectacin (NewBiotics) | (Paligent) |
| | Edotreotid (Novartis) | |
| Farnesyl | Arglabin (NuOncology | Tipifarnib (Johnson & |
| transferase | Labs) | Johnson) |
| inhibitors | Ionafarnib (Schering- | Perillyl alcohol (DOR |
| | Plough) | BioPharma) |
| | BAY-43-9006 (Bayer) | |
| Pump inhibitors | CBT-1 (CBA Pharma) | Zosuquidar |
| | Tariquidar (Xenova) | trihydrochloride (Eli Lilly) |
| | MS-209 (Schering AG) | Biricodar dicitrate (Vertex) |
| Histone acetyl | Tacedinaline (Pfizer) | Pivaloyloxymethyl butyrate |
| transferase inhibitors | SAHA (Aton Pharma) | (Titan) |
| | MS-275 (Schering AG) | Depsipeptide (Fujisawa) |
| Metalloproteinase | Neovastat (Aeterna Laboratories) | CMT-3 (CollaGenex) |
| inhibitors | Marimastat (British Biotech) | BMS-275291 (Celltech) |
| Ribonucleoside | Gallium maltolate (Titan) | Tezacitabine (Aventis) |
| reductase inhibitors | Triapin (Vion) | Didox (Molecules for |
| | | Health) |
| TNF-alpha | Virulizin (Lorus Therapeutics) | Revimid (Celgene) |
| agonists/ | CDC-394 (Celgene) | |
| antagonists | | |
| Endothelin-A receptor | Atrasentan (Abbot) | YM-598 (Yamanouchi) |
| antagonists | ZD-4054 (AstraZeneca) | |
| Retinoic acid receptor | Fenretinide (Johnson & | Alitretinoin (Ligand) |
| agonists | Johnson) | |
| | LGD-1550 (Ligand) | |
| Immunomodulators | Interferon | Dexosome therapy (Anosys) |
| | Oncophage (Antigenics) | Pentrix (Australian Cancer |
| | GMK (Progenics) | Technology) |
| | Adenocarcinoma vaccine | JSF-154 (Tragen) |
| | (Biomira) | Cancer vaccine (Intercell) |
| | CTP-37 (AVI BioPharma) | Norelin (Biostar) |
| | JRX-2 (Immuno-Rx) | BLP-25 (Biomira) |
| | PEP-005 (Peplin Biotech) | MGV (Progenics) |
| | Synchrovax vaccines (CTL | !3-Alethin (Dovetail) |
| | Immuno) | CLL-Thera (Vasogen) |
| | Melanoma vaccine (CTL | |
| | Immuno) | |
| | p21-RAS vaccine | |
| | (GemVax) | |
| Hormonal and | Oestrogens | Prednisone |
| antihormonal | Conjugated oestrogens | Methylprednisolone |
| agents | Ethynyloestradiol | Prednisolone |
| | chlorotrianisene | Aminoglutethimide |
| | Idenestrol | Leuprolide |
| | Hydroxyprogesterone | Goserelin |
| | caproate | Leuporelin |
| | Medroxyprogesterone | Bicalutamide |
| | Testosterone | Flutamide |
| | Testosterone propionate | Octreotide |
| | Fluoxymesterone | Nilutamide |
| | Methyltestosterone | Mitotan |
| | Diethylstilbestrol | P-04 (Novogen) |
| | Megestrol | 2-Methoxyoestradiol (Entre |
| | Tamoxifen | Med) |
| | Toremofin | Arzoxifen (Eli Lilly) |
| | Dexamethasone | |
| Photodynamic | Talaporfin (Light Sciences) | Pd-bacteriopheophorbide |
| agents | Theralux (Theratechnologies) | (Yeda) |
| | Motexafin gadolinium | Lutetium texaphyrin |
| | (Pharmacyclics) | (Pharmacyclics) |
| | | Hypericin |
| Tyrosine kinase | Imatinib (Novartis) | Kahalide F (PharmaMar) |
| inhibitors | Leflunomide (Sugen/ | CEP-701 (Cephalon) |
| | Pharmacia) | CEP-751 (Cephalon) |
| | ZDI839 (AstraZeneca) | MLN518 (Millenium) |
| | Erlotinib (Oncogene | PKC412 (Novartis) |
| | Science) | Phenoxodiol O |
| | Canertjnib (Pfizer) | Trastuzumab (Genentech) |
| | Squalamine (Genaera) | C225 (ImClone) |
| | SU5416 (Pharmacia) | rhu-Mab (Genentech) |
| | SU6668 (Pharmacia) | MDX-H210 (Medarex) |
| | ZD4190 (AstraZeneca) | 2C4 (Genentech) |
| | ZD6474 (AstraZeneca) | MDX-447 (Medarex) |
| | Vatalanib (Novartis) | ABX-EGF (Abgenix) |
| | PKI166 (Novartis) | IMC-1C11 (ImClone) |
| | GW2016 (GlaxoSmith- | |
| | Kline) | |
| | EKB-509 (Wyeth) | |
| | EKB-569 (Wyeth) | |
| Various agents | SR-27897 (CCK-A | BCX-1777 (PNP inhibitor, |
| | inhibitor, Sanofi- | BioCryst) |
| | Synthelabo) | Ranpirnase (ribonuclease |
| | Tocladesine (cyclic AMP | stimulant, Alfacell) |
| | agonist, Ribapharm) | Galarubicin (RNA |
| | Alvocidib (CDK inhibitor, | synthesis inhibitor, |
| | Aventis) | Dong-A) |
| | CV-247 (COX-2 inhibitor, | Tirapazamine (reducing |
| | Ivy Medical) | agent, SRI International) |
| | P54 (COX-2 inhibitor, | N-Acetylcysteine (reducing |
| | Phytopharm) | agent, Zambon) |
| | CapCell ™ (CYP450 | R-Flurbiprofen (NF-kappaB |
| | stimulant, Bavarian | inhibitor, Encore) |
| | Nordic) | 3CPA (NF-kappaB |
| | GCS-IOO (gal3 | inhibitor, Active Biotech) |
| | antagonist, | Seocalcitol (vitamin D |
| | GlycoGenesys) | receptor agonist, Leo) |
| | G17DT immunogen | 131-I-TM-601 (DNA |
| | (gastrin inhibitor, Aphton) | antagonist, |
| | Efaproxiral (oxygenator, | TransMolecular) |
| | Allos Therapeutics) | Eflornithin (ODC inhibitor, |
| | PI-88 (heparanase | ILEX Oncology) |
| | inhibitor, Progen) | Minodronic acid |
| | Tesmilifen (histamine | (osteoclast inhibitor, |
| | antagonist, YM | Yamanouchi) |
| | BioSciences) | Indisulam (p53 stimulant, |
| | Histamine (histamine H2 | Eisai) |
| | receptor agonist, | Aplidin (PPT inhibitor, |
| | Maxim) | PharmaMar) |
| | Tiazofurin (IMPDH | Rituximab (CD20 antibody, |
| | inhibitor, Ribapharm) | Genentech) |
| | Cilengitide (integrin | Gemtuzumab (CD33 |
| | antagonist, Merck KGaA) | antibody, Wyeth Ayerst) |
| | SR-31747 (IL-1 antagonist, | PG2 (haematopoiesis |
| | Sanofi-Synthelabo) | promoter, Pharmagenesis) |
| | CCI-779 (mTOR kinase | Immunol ™ (triclosan |
| | inhibitor, Wyeth) | mouthwash, Endo) |
| | Exisulind (PDE-V inhibitor, | Triacetyluridine (uridine |
| | Cell Pathways) | prodrug, Wellstat) |
| | CP-461 (PDE-V inhibitor, | SN-4071 (sarcoma agent, |
| | Cell Pathways) | Signature BioScience) |
| | AG-2037 (GART inhibitor, | TransMID-107 ™ |
| | Pfizer) | (immunotoxin, KS |
| | WX-UK1 | Biomedix) |
| | (plasminogen activator | PCK-3145 (apoptosis |
| | inhibitor, Wilex) | promoter, Procyon) |
| | PBI-1402 (PMN | Doranidazole (apoptosis |
| | stimulant, ProMetic | promoter, Pola) |
| | LifeSciences) | CHS-828 (cytotoxic agent, |
| | Bortezomib (proteasome | Leo) |
| | inhibitor, Millennium) | trans-Retinoic acid |
| | SRL-172 (T-cell stimulant, | (differentiator, NIH) |
| | SR Pharma) | MX6 (apoptosis promoter, |
| | TLK-286 (glutathione-S | MAXIA) |
| | transferase inhibitor, | Apomine (apoptosis |
| | Telik) | promoter, ILEX Oncology) |
| | PT-100 | Urocidin (apoptosis |
| | (growth factor | promoter, Bioniche) |
| | agonist, Point | Ro-31-7453 (apoptosis |
| | Therapeutics) | promoter, La Roche) |
| | Midostaurin (PKC | Brostallicin (apoptosis |
| | inhibitor, Novartis) | promoter, Pharmacia) |
| | Bryostatin-1 (PKC | |
| | stimulant, GPC Biotech) | |
| | CDA-II (apoptosis | |
| | promoter, Everlife) | |
| | SDX-101 (apoptosis | |
| | promoter, Salmedix) | |
| | Ceflatonin (apoptosis | |
| | promoter, ChemGenex) | |

The compounds of the formula I are preferably combined with the with known anti-cancer agents:

These known anti-cancer agents include the following: oestrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors and other angiogenesis inhibitors. The present compounds are particularly suitable for administration at the same time as radiotherapy. The synergistic effects of inhibition of VEGF in combination with radiotherapy have been described in the art (see WO 00/61186). “Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646. “Androgen receptor modulators” refers to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide and N-4-carboxyphenylretinamide.

“Cytotoxic agents” refers to compounds which result in cell death primarily through direct action on the cellular function or inhibit or interfere with cell myosis, including alkylating agents, tumour necrosis factors, intercalators, microtubulin inhibitors and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cisaminedichloro(2-methylpyridine)platinum, benzylguanine, glufosfamide, GPX100, (trans,trans,trans)bis-mu-(hexane-1,6-diamine)mu-[diamineplatinum(II)]bis[diamine(chloro)platinum(II)]tetrachloride, diarisidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplastone, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzenesulfonamide, anhydrovinblastine, N,N-dimethyl-L-valyl-L-valyl-N-methyl-L-valyl-L-prolyl-L-proline-t-butylamide, TDX258 and BMS188797.

Some examples of topoisomerase inhibitors are topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)-ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those listed under “angiogenesis inhibitors”, such as trastuzumab, and tumour suppressor genes, such as p53, which can be delivered via recombinant virus-mediated gene transfer (see U.S. Pat. No. 6,069,134, for example).

Particular preference is given to the use of the compound according to the invention for the treatment and prophylaxis of tumour diseases.

The tumour is preferably selected from the group of tumours of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx and/or of the lung.

The tumour is furthermore preferably selected from the group lung adenocarcinoma, small-cell lung carcinomas, pancreatic cancer, ovarian carcinoma, glioblastomas, colon carcinoma and breast carcinoma.

Preference is furthermore given to the use for the treatment of a tumour of the blood and immune system, preferably for the treatment of a tumour selected from the group of acute myeloid leukaemia, chronic myeloid leukaemia, acute lymphatic leukaemia and/or chronic lymphatic leukaemia.

In another aspect, the invention encompasses a for the treatment of a patient who has a neoplasm, such as a cancer, by administration of a compound of the formula (I) in combination with an antiproliferative agent. Suitable antiproliferative agents encompass those provided in Table 1.

Above and below, all temperatures are indicated in ° C. In the following examples, “conventional work-up” means: if necessary, water is added, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rt values are determined by HPLC using eluents mentioned.
Mass spectrometry (MS): EI (electron impact ionisation) M⁺
   FAB (fast atom bombardment) (M+H)⁺
   ESI (electrospray ionisation) (M+H)⁺
APCI-MS (atmospheric pressure chemical ionisation—mass spectrometry) (M+H)⁺

### LC/MS Method:

Solvent A: water+0.1% of HCOOH
Solvent B: acetonitrile+0.1% of HCOOH
Flow: 2.4 ml/min
Gradient: 0.0 min 4% of B
   2.6 min 100% of B
Column: Chromolith® Speed ROD RP-18e 50-4, 6 mm

### HPLC Method:

Solvent A: water+0.1% of HCOOH
Solvent B: acetonitrile+0.08% of HCOOH
Flow: 1.5 ml/min
Gradient: 0.0-0.5 min 100% of A
   0.5-3.5 min auf 100% of B
   3.5-4.5 min 100% of B
   4.5-4.6 min auf 100% of A
   4.6-5.0 min 1000% of A
Column: Si-ROD® UM9423/100, 3 mm

### EXAMPLE 1

### Synthesis of N-(5-chloro-2-methoxyphenyl)-2-[10-(4-methylpiperazine-1-carbonyl)-3,4-dihydro-1H-benzo[b][1,6]naphthyridin-2-yl]acetamide (“A1”, rt[min] 2.45)

is carried out analogously to the following scheme

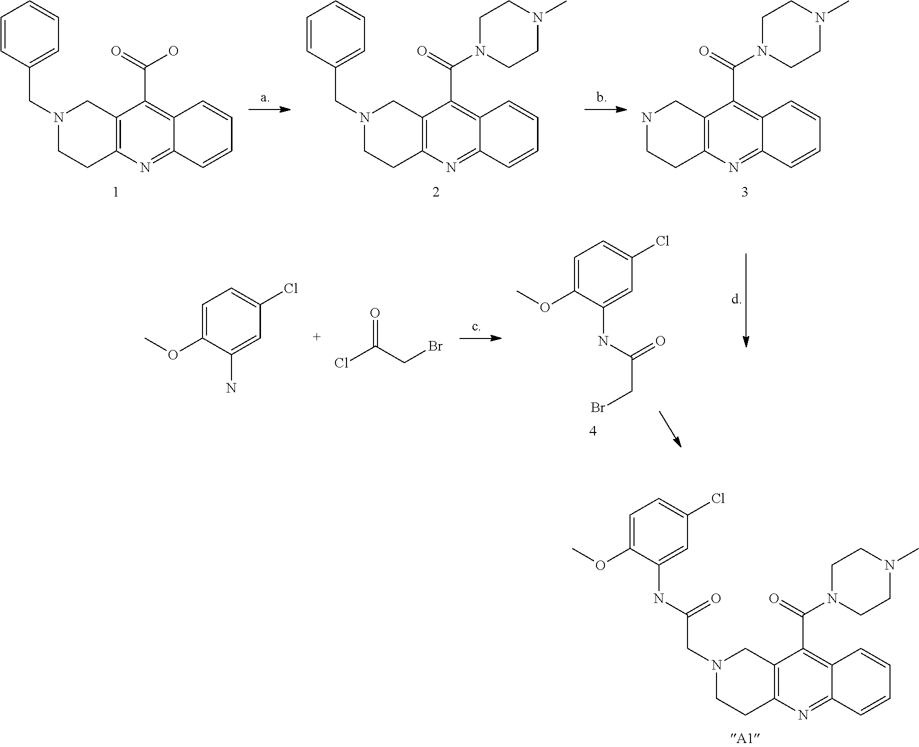

a. 0.48 g (1.5 mmol) of 1 and 0.70 g (1.5 mmol) of PyBroP are dissolved in 20 ml of DMF and stirred for 5 min. 1.67 ml (1.5 mmol) of methylpiperazine are then added, and the mixture is stirred at room temperature (RT) for 2 hours. The solvent is removed in a rotary evaporator, diluted with water (100 ml) and extracted 2× with EA. The organic phase is dried over magnesium sulfate, filtered off and evaporated to dryness. Purification by means of preparative HPLC gives 0.29 g (48.3%) of 2 as colourless amorphous product.

b. 0.26 g (0.65 mmol) of 2 are dissolved in 5 ml of THF, 2 ml of methanol and 0.3 ml of acetic acid (100%), 0.3 g of 5% Pd/C is added, and the mixture is hydrogenated at RT for 32 h. The catalyst is then filtered off, and the solvent is evaporated in vacuo. The residue is purified by means of preparative HPLC, giving 76 mg (37.7%) of starting material 3 as amorphous product.

c. 0.79 g (5 mmol) of 5-chloro-2-methoxyphenylamine are dissolved in 50 ml of DCM. 0.7 ml (5 mmol) of triethylamine are added. 0.42 ml (5 mmol) of bromoacetyl chloride is then added dropwise with ice-cooling. The mixture is then stirred at RT for 2 h. The mixture is washed with water. The organic phase is then dried over sodium sulfate, filtered off, and the solvent is evaporated in vacuo. The starting material (4, 1.2 g, 86%) is reacted further without purification.

d. 72 mg (0.23 mmol) of 3, 65 mg (0.23 mmol) of 4 and 76 mg (0.23 mmol) of caesium carbonate are stirred in 5 ml of DMF for 18 hours. Water is then added to the batch, which is then extracted with glacial acetic acid. The organic phase is dried over magnesium sulfate, filtered off and evaporated to dryness. The residue is purified by means of preparative HPLC, giving 54 mg (46%) of substance “A1” as amorphous product.

1H-NMR (DMSO-d6): d [ppm] for “A1” 2.86 (s, 3H), 3.14-3.57 (m, 9H), 3.68 (m, 1H), 3.90 (m, 5H), 4.46-4.85 (m, 4H), 7.15 (d, 1H), 7.23 (dd, 1H), 7.77 (m, 1H), 7.83-8.03 (m, 2H), 8.10 (m, 1H), 8.15 (d, 1H), 10.20 (s, 1H)*

The following compounds are prepared analogously to Example 1 using the corresponding precursors (3 and 4):

### (2-Benzyl-1,2,3,4-tetrahydrobenzo[b][1,6]naphthyridin-10-yl)-(4-methylpiperazin-1-yl)methanone (“A2”, rt[min] 1.63)

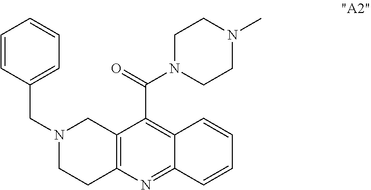

### N-(5-Chloro-2-methoxyphenyl)-2-[10-(4-ethylpiperazine-1-carbonyl)-3,4-dihydro-1H-benzo[b][1,6]naphthyridin-2-yl]acetamide (“A23”, rt[min] 2.37)

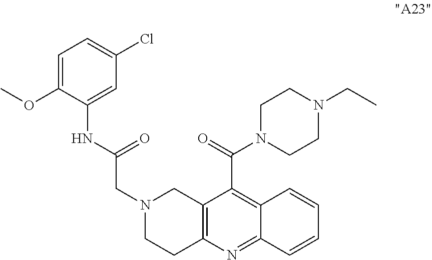

1H-NMR (DMSO-d6): d [ppm] for “A23” 1.25 (m, 3H), 2.97-4.05 (3m, 17H), 4.43-4.88 (m, 4H), 7.12 (d, 1H), 7.21 (dd, 1H), 7.74 (m, 1H), 7.80-8.10 (3m, 3H), 8.13 (d, 1H), 10.20 (s, 1H)*

Compound N-(5-chloro-2-methoxyphenyl)-2-[10-(4-ethylpiperazine-1-carbonyl)-3,4-dihydro-1H-benzo[b][1,6]naphthyridin-2-yl]acetamide (“A24”, rt[min] 2.37)

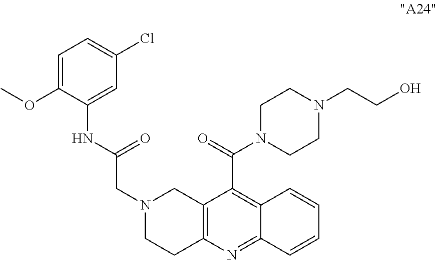

1H-NMR (DMSO-d6): d [ppm] for “A24” 2.11-3.92 (9m, 22H), 4.36 (t, 2H), 7.04 (d, 1H), 7.10 (dd, 1H), 7.63 (m, 2H), 7.77 (dt, 1H), 8.00 (d, 1H), 8.29 (d, 1H), 9.73 (s, 1H)

### 4-{2-[(5-Chloro-2-methoxyphenylcarbamoyl)methyl]-1,2,3,4-tetrahydrobenzo[b][1,6]naphthyridine-10-carbonyl}piperazine-1-carboxylic acid benzyl ester (“25”, rt[min] 4.08)

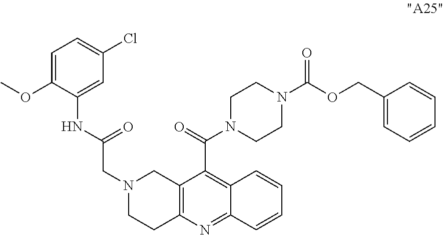

1H-NMR (DMSO-d6): d [ppm] for “A25” 3.11 (m, 3H), 3.37 (m, 2H), 3.53 (m, 1H), 3.75 (m, 2H), 3.80-3.99 (m, 7H), 4.55 dd, 2H), 4.70 (dd, 2H), 5.07 (m, 2H), 7.05 (d, 1H), 7.15 dd, 1H), 7.30 (m, 5H), 7.78 (m, 1H), 7.90 (d, 1H), 7.98 (m, 1H), 8.09 (d, 1H), 8.18 (d, 1H), 8.33 (m, 1H), 10.12 (s, 1H)*

### 2-[10-(4-Benzyl-1,4-diazepan-1-carbonyl)-3,4-dihydro-1H-benzo[b][1,6]-naphthyridin-2-yl]-N-(5-chloro-2-methoxyphenyl)acetamide (“A26”, rt[min] 2.83)

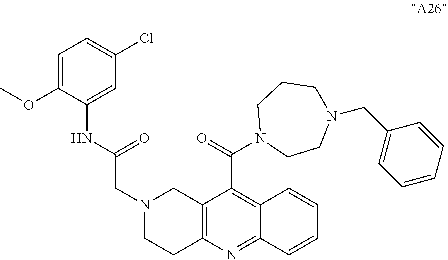

Compound N-(5-chloro-2-methoxyphenyl)-2-[10-(piperazine-1-carbonyl)-3,4-dihydro-1H-benzo[b][1,6]naphthyridin-2-yl]acetamide (“A27”, rt[min] 2.37)

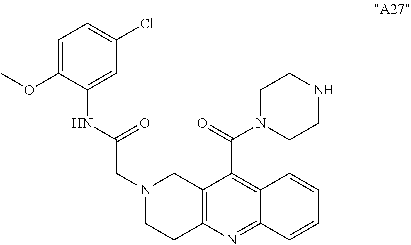

### N-(5-Chloro-2-methoxyphenyl)-2-[10-(4-cyclopentylpiperazine-1-carbonyl)-3,4-dihydro-1H-benzo[b][1,6]naphthyridin-2-yl]acetamide (“A28”, rt[min] 2.64)

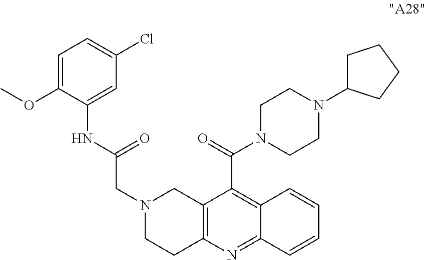

1H-NMR (DMSO-d6): d [ppm] for “A28” 1.43-2.11 (4m, 12H), 2.72-3.96 (6m, 12H), 4.43-4.85 (m, 4H), 7.08 (d, 1H), 7.17 (m, 1H), 7.74 (m, 1H), 7.83 (m, 1H), 7.92 (m, 1H), 8.00 (m, 1H), 8.10 (d, 1H), 9.75 (s, 1H)*

### N-(5-Chloro-2-methoxyphenyl)-2-[10-(4-propylpiperazine-1-carbonyl)-3,4-dihydro-1H-benzo[b][1,6]naphthyridin-2-yl]acetamide (“A29”, rt[min] 2.48)

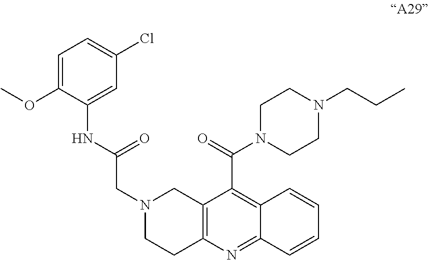

1H-NMR (DMSO-d6): d [ppm] for “A29” 0.87 (m, 3H), 1.65 (m, 2H), 2.93-3.97 (5m, 17H), 4.47-4.82 (m, 4H), 7.08 (d, 1H), 7.17 (m, 1H), 7.75 (m, 1H), 7.86 (m, 1H), 7.93 (m, 1H), 8.04 (m, 1H), 8.13 (d, 1H), 9.78 (s, 1H)*

### 2-[10-(4-Benzylpiperazine-1-carbonyl)-3,4-dihydro-1H-benzo[b][1,6]naphthyridin-2-yl]-N-(5-chloro-2-methoxyphenyl)acetamide (“A30”, rt[min] 3.01)

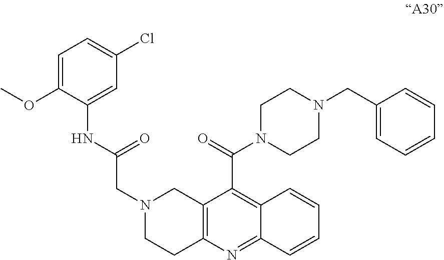

### {2-[(5-Chlorobenzofuran-7-ylamino)methyl]-1,2,3,4-tetrahydrobenzo[b][1,6]-naphthyridin-10-yl}-[4-(2-hydroxyethyl)piperazin-1-yl]methanone (“A57”, rt[min] 2.40)

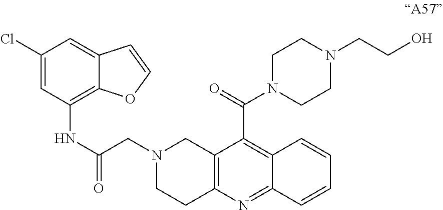

### 4-Chloro-2-({10-[4-(2-hydroxyethyl)piperazine-1-carbonyl]-3,4-dihydro-1H-benzo[b][1,6]naphthyridin-2-ylmethyl}amino)benzonitrile (“A58”, rt[min] 2.27)

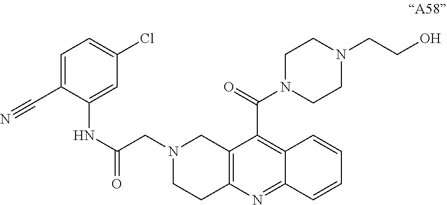

### 4-Chloro-2-({10-[4-(2-hydroxyethyl)piperazine-1-carbonyl]-3,4-dihydro-1H-benzo[b][1,6]naphthyridin-2-ylmethyl}amino)benzoic acid methyl ester (“A59”, rt[min] 2.75)

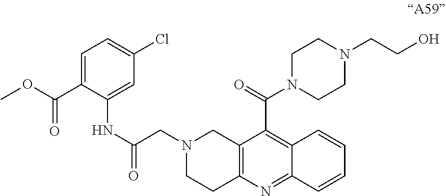

### N-(5-Chloro-2-isopropoxyphenyl)-2-{10-[4-(2-hydroxyethyl)piperazine-1-carbonyl]-3,4-dihydro-1H-benzo[b][1,6]naphthyridin-2-yl}acetamide (“A60”, rt[min] 2.88)

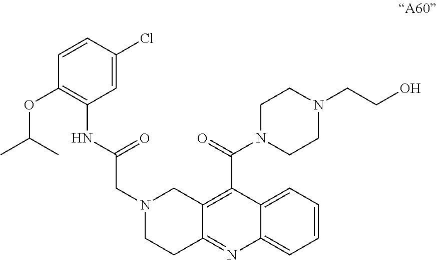

### N-[5-Chloro-2-(2-hydroxyethoxy)phenyl]-2-{10-[4-(2-hydroxyethyl)-piperazine-1-carbonyl]-3,4-dihydro-1H-benzo[b][1,6]naphthyridin-2-yl}-acetamide (“A61”, rt[min] 2.16)

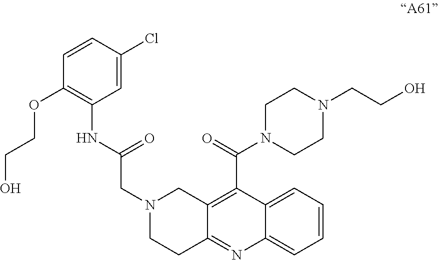

### N-(5-Fluoro-2-methoxyphenyl)-2-{10-[4-(2-hydroxyethyl)piperazine-1-carbonyl]-3,4-dihydro-1H-benzo[b][1,6]naphthyridin-2-yl}acetamide (“A62”, rt[min] 2.24)

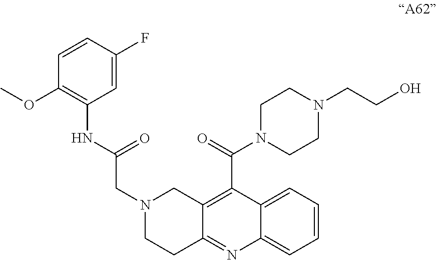

### 4-Chloro-2-(2-{10-[4-(2-hydroxyethyl)piperazine-1-carbonyl]-3,4-dihydro-1H-benzo[b][1,6]naphthyridin-2-yl}acetylamino)benzoic acid (“A63”, rt[min] 2.27)

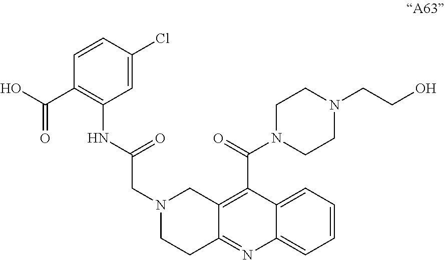

### N-[3-Chloro-4-(2-oxopyrrolidin-1-yl)phenyl]-2-{10-[4-(2-hydroxyethyl)-piperazine-1-carbonyl]-3,4-dihydro-1H-benzo[b][1,6]naphthyridin-2-yl}-acetamide (“A64”, rt[min] 1.95)

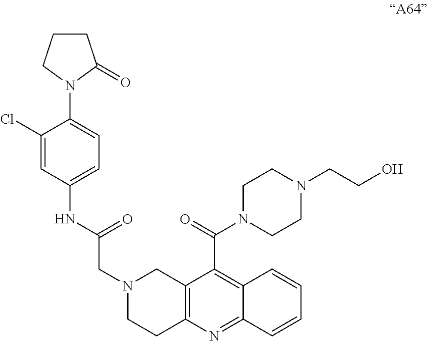

### N-[3-Chloro-4-(3-oxomorpholin-4-yl)phenyl]-2-{10-[4-(2-hydroxyethyl)-piperazine-1-carbonyl]-3,4-dihydro-1H-benzo[b][1,6]naphthyridin-2-yl}-acetamide (“A65”, rt[min] 1.87)

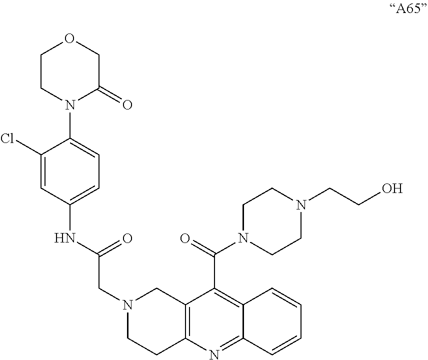

### N-(5-Chloro-2-methoxyphenyl)-2-{10-[4-(2-piperidin-1-ylethyl)piperazine-1-carbonyl]-3,4-dihydro-1H-benzo[b][1,6]naphthyridin-2-yl}acetamide (“A66”, rt[min] 2.61)

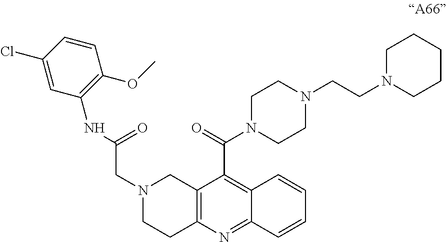

### N-(5-Chloro-2-methoxyphenyl)-2-{10-[4-(2-dimethylaminoethyl)piperazine-1-carbonyl]-3,4-dihydro-1H-benzo[b][1,6]naphthyridin-2-yl}acetamide (A“67”, rt[min] 2.53)

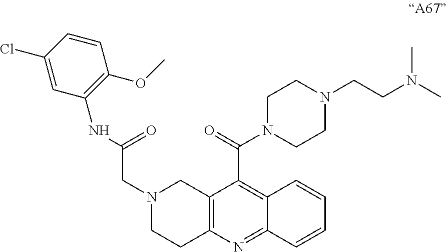

### N-(5-Chloro-2-methoxyphenyl)-2-{10-[4-(2-morpholin-4-ylethyl)piperazine-1-carbonyl]-3,4-dihydro-1H-benzo[b][1,6]naphthyridin-2-yl}acetamide (“A68”, rt[min] 2.48)

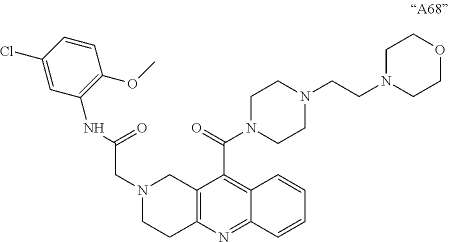

### N-(5-Bromo-2-methoxyphenyl)-2-{10-[4-(2-hydroxyethyl)piperazine-1-carbonyl]-3,4-dihydro-1H-benzo[b][1,6]naphthyridin-2-yl}acetamide (“A69”, rt[min] 2.53)

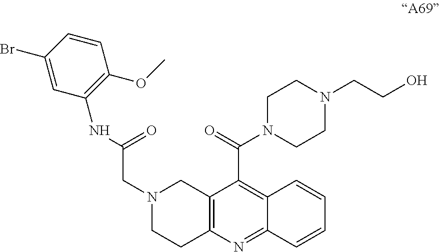

### 1-(6-Chloro-2,3-dihydrobenzo-1,4-oxazin-4-yl)-2-{10-[4-(2-hydroxyethyl)-piperazine-1-carbonyl]-3,4-dihydro-1H-benzo[b][1,6]naphthyridin-2-yl}-ethanone (“A70”, rt[min] 2.21)

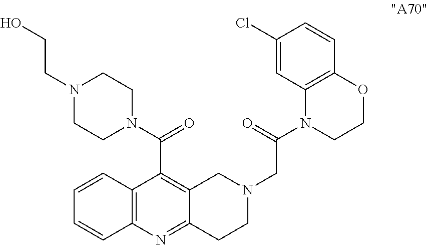

### N-(5-Chloro-2,4-dimethoxyphenyl)-2-{10-[4-(2-hydroxyethyl)piperazine-1-carbonyl]-3,4-dihydro-1H-benzo[b][1,6]naphthyridin-2-yl}acetamide (“A71”, rt[min] 2.38)

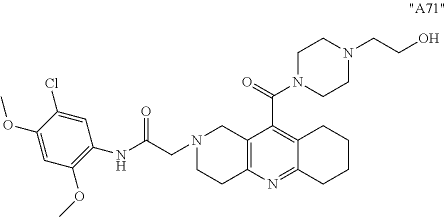

### N-(3-Chloro-4-methoxyphenyl)-2-{10-[4-(2-hydroxyethyl)piperazine-1-carbonyl]-3,4-dihydro-1H-benzo[b][1,6]naphthyridin-2-yl}acetamide (“A72”, rt[min] 2.31)

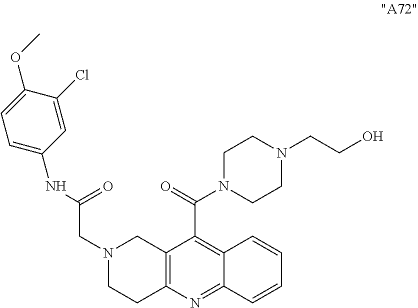

### N-(3-Chloro-4-methylphenyl)-2-{10-[4-(2-hydroxyethyl)piperazine-1-carbonyl]-3,4-dihydro-1H-benzo[b][1,6]naphthyridin-2-yl}acetamide (“A73”, rt[min] 2.44)

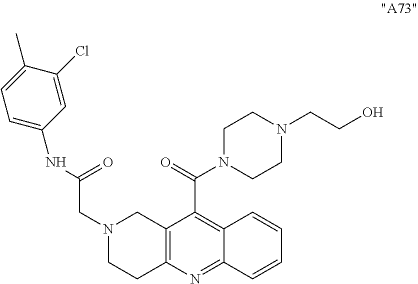

### N-(3-Chloro-4-fluorophenyl)-2-{10-[4-(2-hydroxyethyl)piperazine-1-carbonyl]-3,4-dihydro-1H-benzo[b][1,6]naphthyridin-2-yl}acetamide (“A74”, rt[min] 2.43)

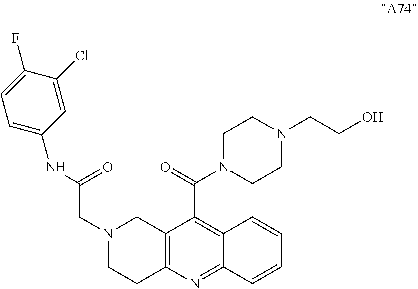

### N-(2,5-Dichlorophenyl)-2-{10-[4-(2-hydroxyethyl)piperazine-1-carbonyl]-3,4-dihydro-1H-benzo[b][1,6]naphthyridin-2-yl}acetamide (“A75”, rt[min] 2.42)

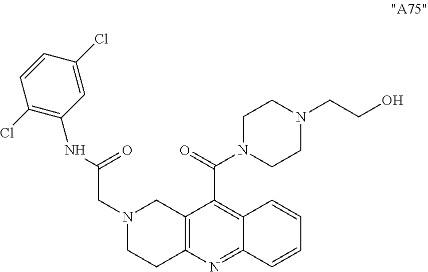

### N-(3,4-Dichlorophenyl)-2-{10-[4-(2-hydroxyethyl)piperazine-1-carbonyl]-3,4-dihydro-1H-benzo[b][1,6]naphthyridin-2-yl}acetamide (“A76”, rt[min] 2.52)

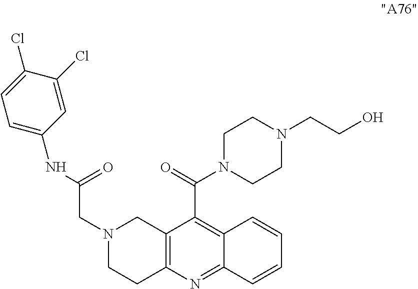

### N-(3-Chloro-2-fluorophenyl)-2-{10-[4-(2-hydroxyethyl)piperazine-1-carbonyl]-3,4-dihydro-1H-benzo[b][1,6]naphthyridin-2-yl}acetamide (“A77”, rt[min] 2.38)

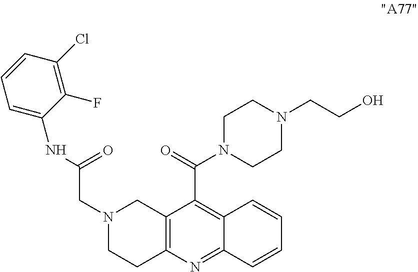

### N-(5-Chloro-2-fluorophenyl)-2-{10-[4-(2-hydroxyethyl)piperazine-1-carbonyl]-3,4-dihydro-1H-benzo[b][1,6]naphthyridin-2-yl}acetamide (“A78”, rt[min] 2.35)

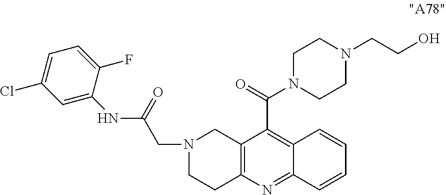

### N-(5-Chlorobenzooxazol-7-yl)-2-{10-[4-(2-hydroxyethyl)piperazine-1-carbonyl]-3,4-dihydro-1H-benzo[b][1,6]naphthyridin-2-yl}acetamide (“A79”, rt[min] 2.21)

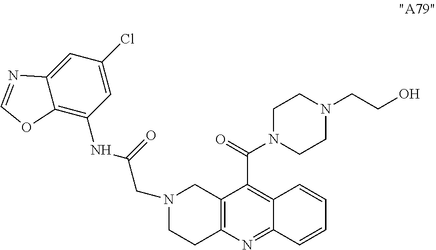

### N-(3,5-Dichloro-2-methoxyphenyl)-2-{10-[4-(2-hydroxyethyl)piperazine-1-carbonyl]-3,4-dihydro-1H-benzo[b][1,6]naphthyridin-2-yl}acetamide (“A80”, rt[min] 2.46)

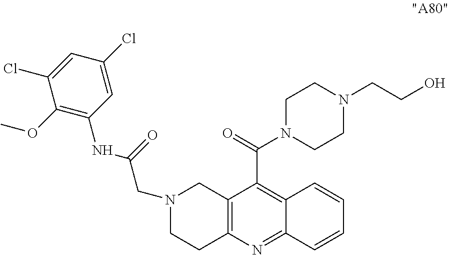

### N-(5-Chloro-2-oxo-2,3-dihydrobenzooxazol-7-yl)-2-{10-[4-(2-hydroxyethyl)-piperazine-1-carbonyl]-3,4-dihydro-1H-benzo[b][1,6]naphthyridin-2-yl}-acetamide (“A81”, rt[min] 2.11)

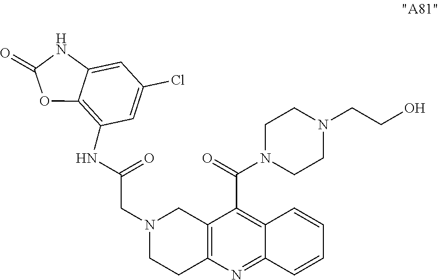

### N-(6-Chloro-3H-benzotriazol-4-yl)-2-{10-[4-(2-hydroxyethyl)piperazine-1-carbonyl]-3,4-dihydro-1H-benzo[b][1,6]naphthyridin-2-yl}acetamide (“A82”, rt[min] 2.14)

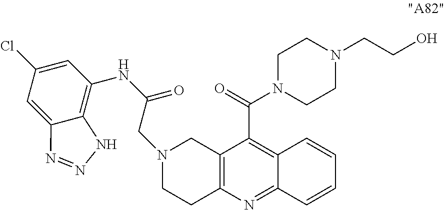

### EXAMPLE 2

### Synthesis of N-(5-chloro-2-methoxyphenyl)-2-[10-(morpholine-4-carbonyl)-3,4-dihydro-1H-benzo[b][1,6]naphthyridin-2-yl]acetamide (“A7”) is carried out analogously to the following scheme

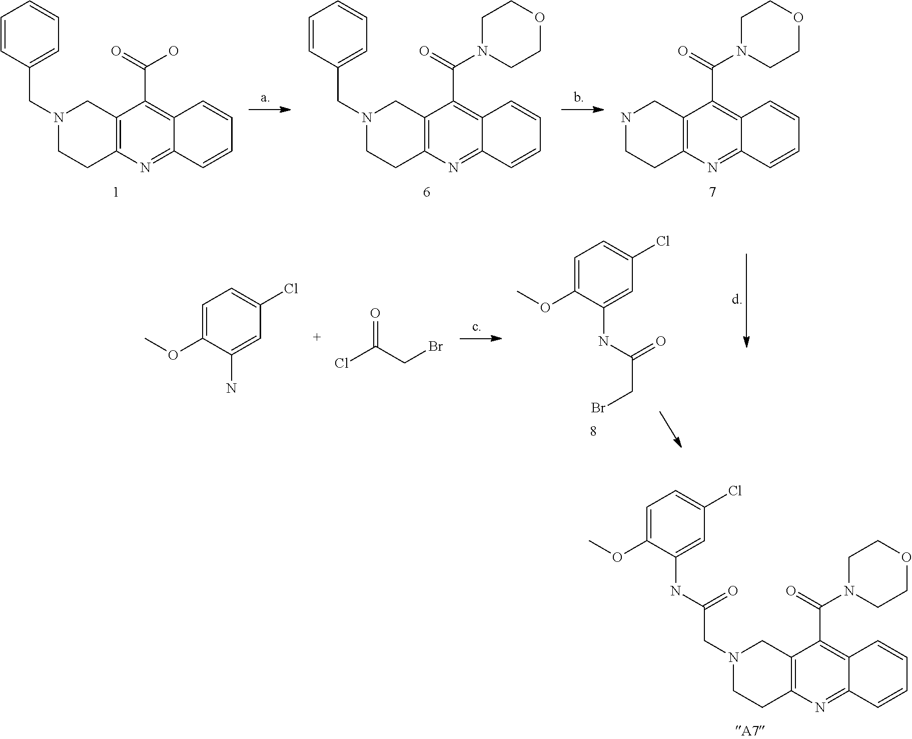

1H-NMR (DMSO-d6): d [ppm] for “A7” 3.07 (m, 4H), 3.43-3.59 (m, 4H), 3.77 (dt, 2H), 3.82-3.95 (m, 5H), 4.50-4.64 (m, 3H), 4.78 (m, 1H), 7.15 (d, 1H), 7.23 (dd, 1H), 7.81 (m, 1H), 7.91 (d, 1H), 7.99 (m, 1H), 8.16 (m, 2H), 10.08 (s, 1H)*

The following compounds are prepared analogously to Example 2 using the corresponding precursor (8):

### (2-Benzyl-1,2,3,4-tetrahydrobenzo[b][1,6]naphthyridin-10-yl)morpholin-4-ylmethanone (“A8”, rt[min] 1.80)

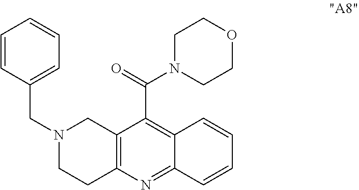

1H-NMR (DMSO-d6): d [ppm] for “A8”) 2.89-3.05 (m, 3H), 3.38-3.53 (m, 3H), 3.60 (m, 1H), 3.73-3.93 (m, 5H), 4.28 (m, 1H), 4.58 (dd, 2H), 4.72 (d, 1H), 7.52 (m, 3H), 7.62 (m, 2H), 7.73 (t, 1H), 7.80 (d, 1H), 7.91 (dt, 1H), 8.10 (d, 1H)*

### N-(3-Chlorophenyl)-2-[10-(morpholine-4-carbonyl)-3,4-dihydro-1H-benzo-[b][1,6]naphthyridin-2-yl]acetamide (“A9”, rt[min] 2.96)

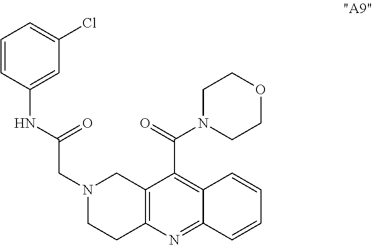

### N-(2-Methoxyphenyl)-2-[10-(morpholine-4-carbonyl)-3,4-dihydro-1H-benzo-[b][1,6]naphthyridin-2-yl]acetamide (“A10”, rt[min] 2.85)

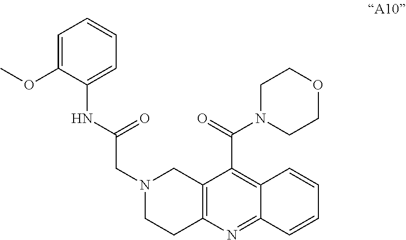

### N-(5-Chloro-2-methylphenyl)-2-[10-(morpholine-4-carbonyl)-3,4-dihydro-1H-benzo[b][1,6]naphthyridin-2-yl]acetamide (“A11”, rt[min] 3.07)

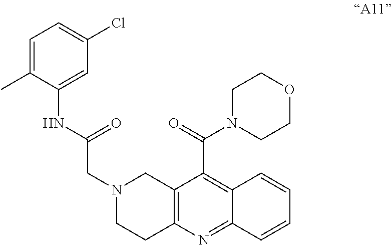

1H-NMR (DMSO-d6): d [ppm] for “A11”) 2.24 (s, 3H), 3.08 (m, 2H), 3.51 (m, 2H), 3.70-3.97 (m, 8H), 4.55 (dd, 2H), 4.72 (dd, 2H), 7.20 (d, 1H), 7.28 (d, 1H), 7.65 (s, 1H), 7.76 (t, 1H), 7.87 (d, 1H), 7.94 (t, 1H), 8.13 (d, 2H), 9.05 (s, 1H)*

### N-(2-Methoxy-5-methylphenyl)-2-[10-(morpholine-4-carbonyl)-3,4-dihydro-1H-benzo[b][1,6]naphthyridin-2-yl]acetamide (“A12”, rt[min] 3.07)

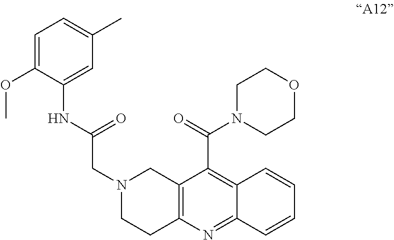

### N-(5-Bromo-2-methoxyphenyl)-2-[10-(morpholine-4-carbonyl)-3,4-dihydro-1H-benzo[b][1,6]naphthyridin-2-yl]acetamide (“A13”, rt[min] 3.39)

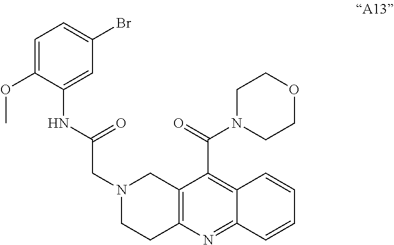

1H-NMR (DMSO-d6): d [ppm] for “A13”) 3.07 (m, 4H), 3.43-3.58 (m, 4H), 3.76 (m, 2H), 3.81-3.99 (m, 5H), 4.47-4.79 (m, 4H), 7.06 (d, 1H), 7.32 (dd, 1H), 7.76 (m, 1H), 7.86 (d, 1H), 7.94 (m, 1H), 8.14 (d, 1H), 8.23 (m, 1H), 10.08 (s, 1H)*

### N-(4-Methoxybiphenyl-3-yl)-2-[10-(morpholine-4-carbonyl)-3,4-dihydro-1H-benzo[b][1,6]naphthyridin-2-yl]acetamide (“A14”, rt[min] 3.71)

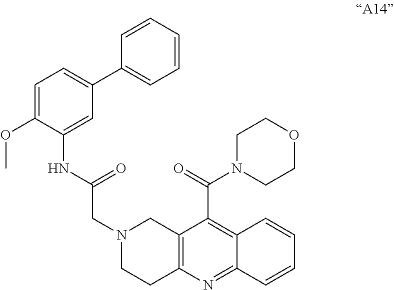

1H-NMR (DMSO-d6): d [ppm] for “A14”) 3.09 (m, 4H), 3.46-3.62 (m, 2H), 3.74 (m, 2H), 3.79-4.00 (m, 7H), 4.58 dd, 2H), 4.73 (dd, 2H), 7.20 (d, 1H), 7.32 (m, 1H), 7.44 (m, 3H), 7.58 (m, 2H), 7.76 (m, 1H), 7.86 (d, 1H), 7.94 (m, 1H), 8.14 (d, 1H), 8.33 (m, 1H), 10.11 (s, 1H)*

### N-(5-Chloro-2-ethoxyphenyl)-2-[10-(morpholine-4-carbonyl)-3,4-dihydro-1H-benzo[b][1,6]naphthyridin-2-yl]acetamide (“A41”)

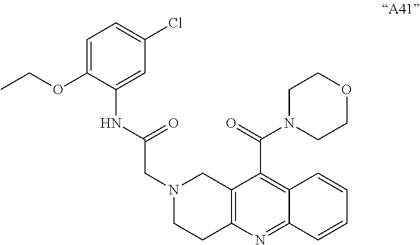

### N-(5-Bromobenzofuran-7-yl)-2-[10-(morpholine-4-carbonyl)-3,4-dihydro-1H-benzo[b][1,6]naphthyridin-2-yl]acetamide (“A43”, rt[min] 3.36)

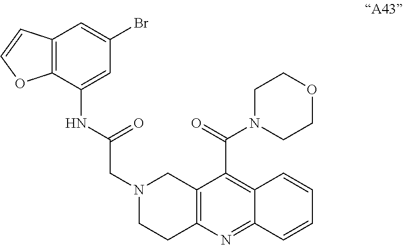

### 2-[10-(Morpholine-4-carbonyl)-3,4-dihydro-1H-benzo[b][1,6]naphthyridin-2-yl]-N-(2-trifluoromethoxy-5-trifluoromethylphenyl)acetamide (“A44”, rt[min] 3.92)

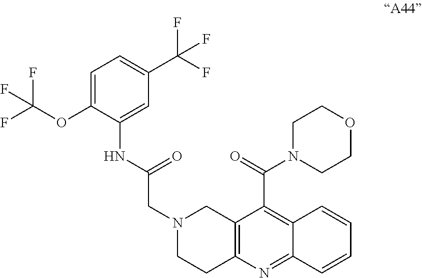

### N-Benzofuran-7-yl-2-[10-(morpholine-4-carbonyl)-3,4-dihydro-1H-benzo-[b][1,6]naphthyridin-2-yl]acetamide (“A45”, rt[min] 2.80)

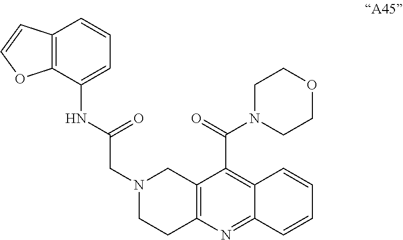

### N-(2,3-Dihydrobenzofuran-7-yl)-2-[10-(morpholine-4-carbonyl)-3,4-dihydro-1H-benzo[b][1,6]naphthyridin-2-yl]acetamide (“A46”, rt[min] 2.61)

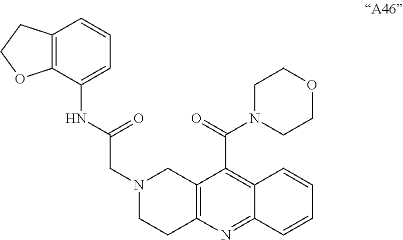

### N-(4-Chloropyridin-2-yl)-2-[10-(morpholine-4-carbonyl)-3,4-dihydro-1H-benzo[b][1,6]naphthyridin-2-yl]acetamide (“A47”, rt[min] 2.75)

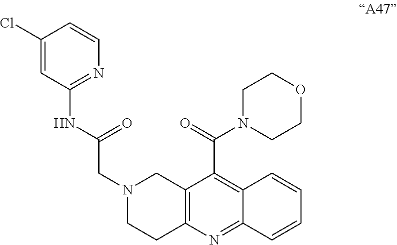

### N-(5-Chlorobenzofuran-7-yl)-2-[10-(morpholine-4-carbonyl)-3,4-dihydro-1H-benzo[b][1,6]naphthyridin-2-yl]acetamide (“A48”, rt[min] 3.33)

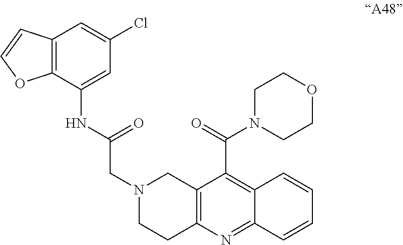

### N-(5-Chloro-2-isopropoxyphenyl)-2-[10-(morpholine-4-carbonyl)-3,4-dihydro-1H-benzo[b][1,6]naphthyridin-2-yl]acetamide (“A49”, rt[min] 3.81)

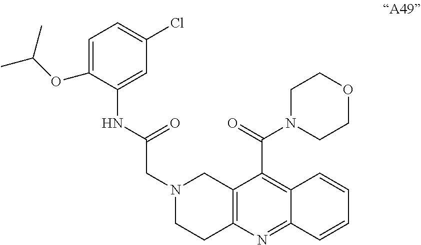

### N-(5-Chloropyridin-3-yl)-2-[10-(morpholine-4-carbonyl)-3,4-dihydro-1H-benzo[b][1,6]naphthyridin-2-yl]acetamide (“A50”, rt[min] 2.40)

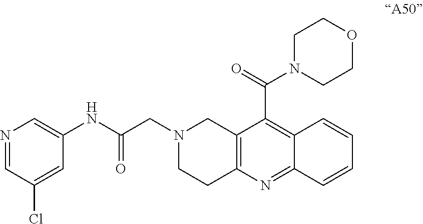

### N-(5-Chloro-2-methoxypyridin-3-yl)-2-[10-(morpholine-4-carbonyl)-3,4-dihydro-1H-benzo[b][1,6]naphthyridin-2-yl]acetamide (“A51”, rt[min] 3.09)

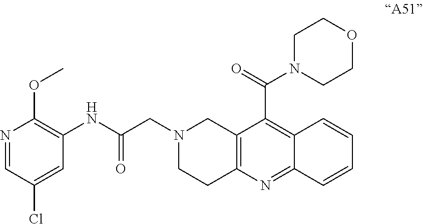

### N-(5-Chloro-2-ethoxypyridin-3-yl)-2-[10-(morpholine-4-carbonyl)-3,4-dihydro-1H-benzo[b][1,6]naphthyridin-2-yl]acetamide (“A52”, rt[min] 3.52)

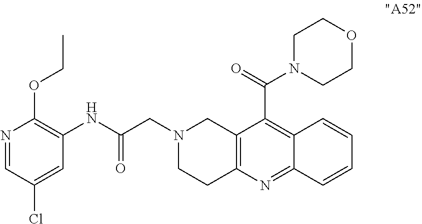

### N-(4-Chloro-2-hydroxyphenyl)-2-[10-(morpholine-4-carbonyl)-3,4-dihydro-1H-benzo[b][1,6]naphthyridin-2-yl]acetamide (“A53”, rt[min] 2.99)

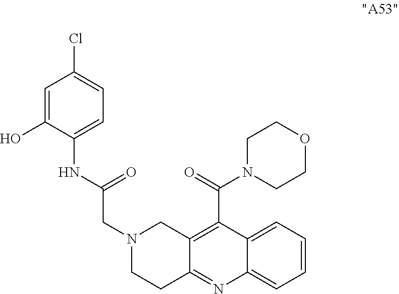

### N-[5-Chloro-2-(2-hydroxyethoxy)phenyl]-2-[10-(morpholine-4-carbonyl)-3,4-dihydro-1H-benzo[b][1,6]naphthyridin-2-yl]acetamide (“A54”, rt[min] 2.83)

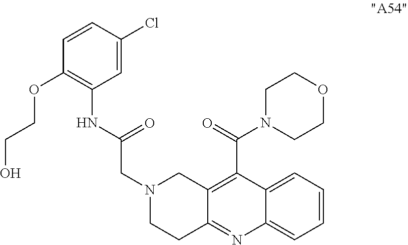

The following compounds are prepared analogously to Example 2 using the corresponding precursors (7 and 8):

### N-(5-Chloro-2-methoxyphenyl)-2-[7-chloro-10-(morpholine-4-carbonyl)-3,4-dihydro-1H-benzo[b][1,6]naphthyridin-2-yl]acetamide (“A15”, rt[min] 3.95)

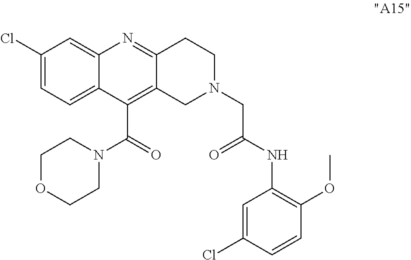

1H-NMR (DMSO-d6): d [ppm] for “A15” 3.09 (m, 2H), 3.53 (m, 2H), 3.77 (m, 2H), 3.84-3.97 (m, 9H), 4.51-4.85 (m, 4H), 7.14 (d, 1H), 7.22 (dd, 1H), 7.71 (dd, 1H), 7.85 (d, 1H), 8.13 (m, 2H), 10.08 (s, 1H)*

### N-(5-Chloro-2-methoxyphenyl)-2-[6-chloro-10-(morpholine-4-carbonyl)-3,4-dihydro-1H-benzo[b][1,6]naphthyridin-2-yl]acetamide (“A16”, rt[min] 3.87)

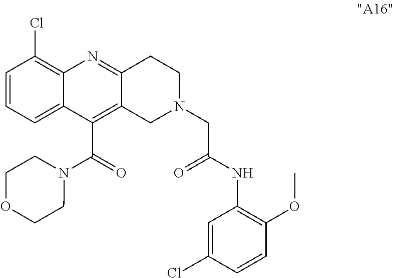

1H-NMR (DMSO-d6): d [ppm] for “A16” 3.05 (m, 2H), 3.41-3.57 (m, 4H), 3.74 (m, 2H), 3.81 (m, 1H), 3.84-3.97 (m, 6H), 4.48-4.82 (m, 4H), 7.10 (d, 1H), 7.18 (dd, 1H), 7.61 (t, 1H), 7.73 (d, 1H), 7.99 (dd, 1H), 8.11 (d, 1H), 10.11 (s, 1H)*

### N-(5-Chloro-2-methoxyphenyl)-2-[7-ethyl-10-(morpholine-4-carbonyl)-3,4-dihydro-1H-benzo[b][1,6]naphthyridin-2-yl]acetamide (“A17”, rt[min] 3.60)

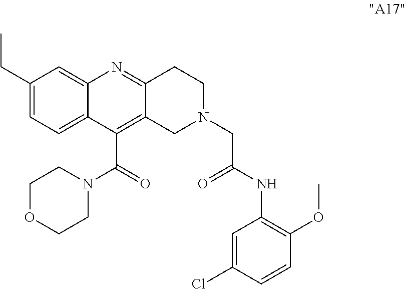

1H-NMR (DMSO-d6): d [ppm] for “A17” 1.30 (t, 3H), 2.91 (dd, 2H), 3.08 (m, 2H), 3.46-3.96 (5m, 13H), 4.49-4.79 (m, 4H), 7.10 (d, 1H), 7.19 (dd, 1H), 7.72 (d, 1H), 7.86 (d, 1H), 8.01 (s, 1H), 8.10 (d, 1H), 10.05 (s, 1H)*

### 3-{2-[10-(Morpholine-4-carbonyl)-3,4-dihydro-1H-benzo[b][1,6]naphthyridin-2-yl]-2-oxoethylamino}benzonitrile (“A21”, rt[min] 3.33)

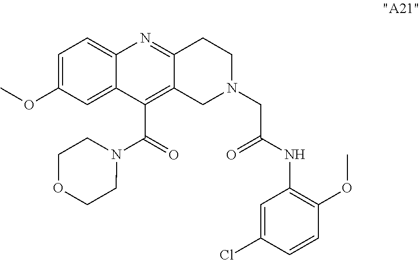

1H-NMR (DMSO-d6): d [ppm] for “A21” 3.04 (m, 4H), 3.19 (m, 2H), 3.36-3.55 (m, 4H), 3.68-3.93 (m, 12H), 6.86 (d, 1H), 7.06 (d, 1H), 7.12 (dd, 1H), 7.42 (d, 1H), 7.92 (d, 1H), 8.28 (d, 1H), 9.70 (s, 1H)

### 2-[6-Bromo-10-(morpholine-4-carbonyl)-3,4-dihydro-1H-benzo[b][1,6]naphthyridin-2-yl]-N-(5-chloro-2-methoxyphenyl)acetamide (“A40”)

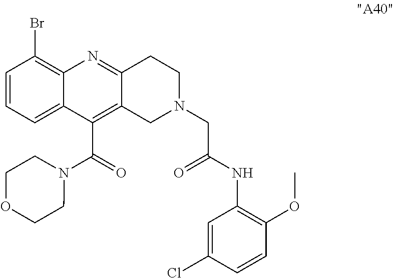

### 2-[7-Bromo-10-(morpholine-4-carbonyl)-3,4-dihydro-1H-benzo[b][1,6]-naphthyridin-2-yl]-N-(5-chloro-2-methoxyphenyl)acetamide (“A42”, rt[min] 3.84)

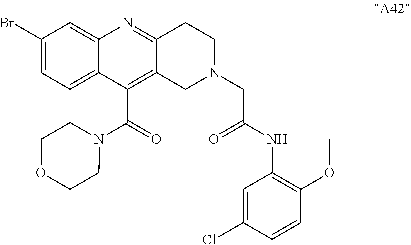

### 1-(6-Chloro-2,3-dihydroindol-1-yl)-2-[10-(morpholine-4-carbonyl)-3,4-dihydro-1H-benzo[b][1,6]naphthyridin-2-yl]ethanone (“A83”, rt[min] 2.67)

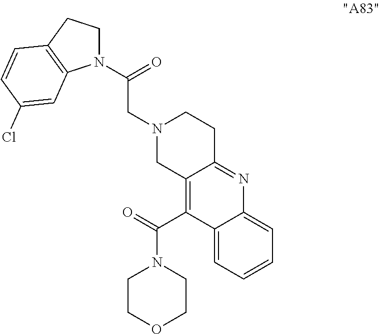

### 1-(3,4-Dihydro-2H-quinolin-1-yl)-2-[10-(morpholine-4-carbonyl)-3,4-dihydro-1H-benzo[b][1,6]naphthyridin-2-yl]ethanone (“A84”, rt[min] 2.52)

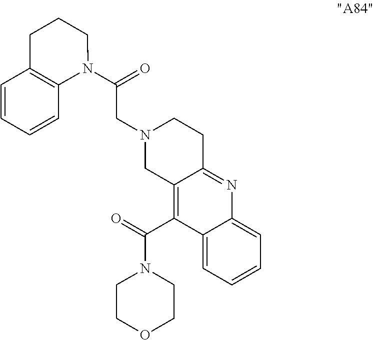

### 1-(2,3-Dihydrobenzo-1,4-oxazin-4-yl)-2-[10-(morpholine-4-carbonyl)-3,4-dihydro-1H-benzo[b][1,6]naphthyridin-2-yl]ethanone (“A85”, rt[min] 2.37)

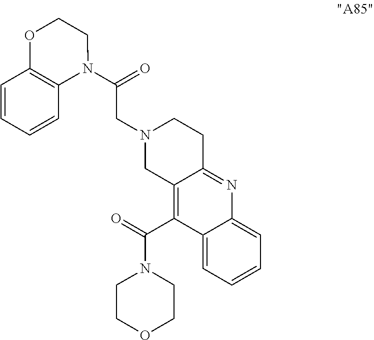

### N-(5-Chloro-2-methoxyphenyl)-N-methyl-2-[10-(morpholine-4-carbonyl)-3,4-dihydro-1H-benzo[b][1,6]naphthyridin-2-yl]acetamide (“A86”, rt[min] 2.51)

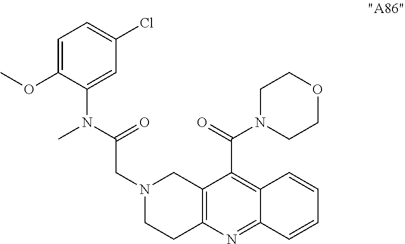

### N-(5-Chloro-2-methoxyphenyl)-N-methyl-2-[10-(morpholine-4-carbonyl)-3,4-dihydro-1H-benzo[b][1,6]naphthyridin-2-yl]acetamide (“A87”, rt[min] 2.46)

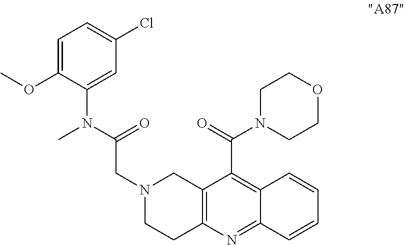

### EXAMPLE 3

### Synthesis of 3-(2,4-dichlorophenyl)-1-[10-(4-methylpiperazine-1-carbonyl)-3,4-dihydro-1H-benzo[b][1,6]naphthyridin-2-yl]propan-1-one (“A31”, rt[min] 2.75)

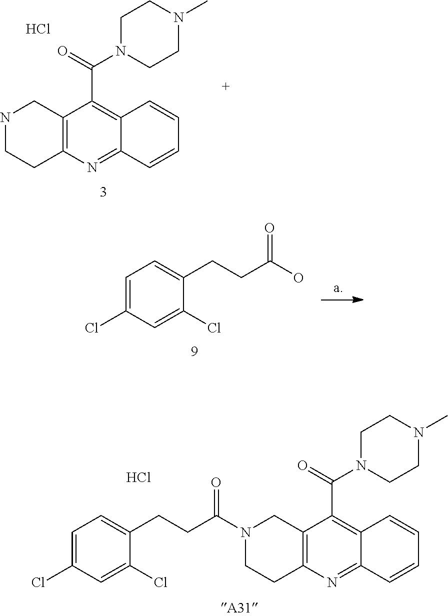

0.17 g (0.5 mmol) of 3 (synthesis is described in Example 1), 0.11 g (0.5 mmol) of 2,4-dichlorophenylpropionic acid, 96 mg (0.5 mmol) of N-(dimethylaminopropyl)-N′-ethylcarbodiimide hydrochloride, 68 mg (0.5 mmol) of HOBt and 0.07 ml (0.5 mmol) of triethylamine are dissolved in 5 ml of DMF and stirred at RT for 20 h. The solvent is then evaporated in a rotary evaporator. The residue is taken up in aqueous Na₂CO₃ solution and EA and extracted by shaking. The organic phase is separated off and dried over magnesium sulfate, filtered off and evaporated to dryness. The residue is dissolved in 1n HCl and freeze-dried, giving 0.18 g (66%) of 6 as colourless amorphous product.

The following compounds are prepared analogously to Example 3 using the corresponding precursor (9):

### 1-[10-(4-Methylpiperazine-1-carbonyl)-3,4-dihydro-1H-benzo[b][1,6]-naphthyridin-2-yl]-2-phenylaminoethanone (“A3”, rt[min] 2.11)

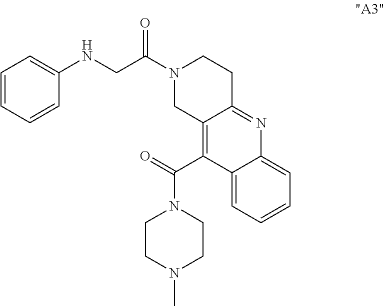

### 2-(2-Chloro-5-methoxyphenylamino)-1-[10-(4-methylpiperazine-1-carbonyl)-3,4-dihydro-1H-benzo[b][1,6]naphthyridin-2-yl]ethanone (“A4”, rt[min] 2.53)

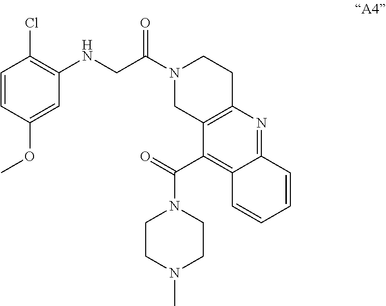

### 4-{2-[10-(4-Methylpiperazine-1-carbonyl)-3,4-dihydro-1H-benzo[b][1,6]-naphthyridin-2-yl]-2-oxoethylamino}benzonitrile (“A5”, rt[min] 2.11)

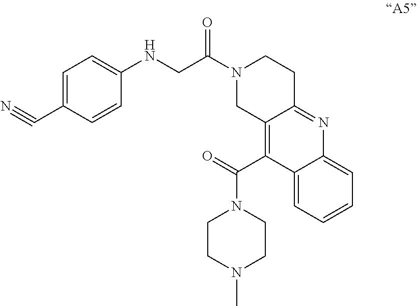

### 3-{2-[10-(4-Methylpiperazine-1-carbonyl)-3,4-dihydro-1H-benzo[b][1,6]-naphthyridin-2-yl]-2-oxoethylamino}benzonitrile (“A6”, rt[min] 2.21)

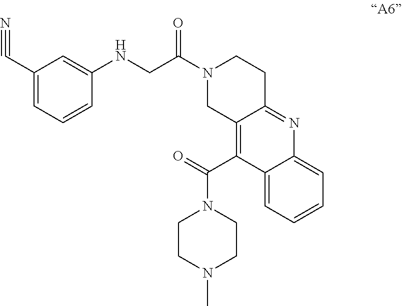

### 3-(2,5-Dimethoxyphenyl)-1-[10-(4-methylpiperazine-1-carbonyl)-3,4-dihydro-1H-benzo[b][1,6]naphthyridin-2-yl]propan-1-one (“A32”, rt[min] 2.27)

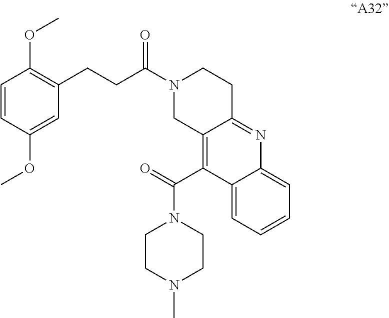

### 3-(4-Chloro-2-fluorophenyl)-1-[10-(4-methylpiperazine-1-carbonyl)-3,4-dihydro-1H-benzo[b][1,6]naphthyridin-2-yl]propan-1-one (“A33”, rt[min] 2.59)

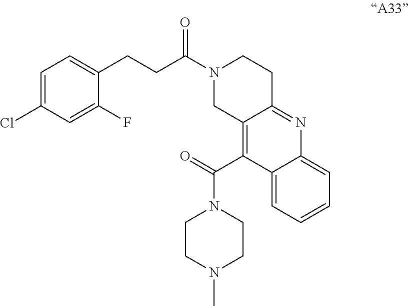

1H-NMR (DMSO-d6): d [ppm] for “A33” 2.74-2.95 (m, 8H), 3.05 (m, 1H), 3.15-3.52 (m, 6H), 3.66 (m, 1H), 3.90 (m, 1H), 4.06 (m, 1H), 4.67-5.02 (m, 3H), 7.16 (m, 1H), 7.25 (m, 1H), 7.35 (m, 1H) 7.90 (t, 1H), 7.99-8.15 (m, 2H), 8.24 (d, 1H)*

### EXAMPLE 4

### Synthesis of 3-(2,4-dichlorophenyl)-1-[10-(morpholine-4-carbonyl)-3,4-dihydro-1H-benzo[b][1,6]naphthyridin-2-yl]propan-1-one (“A35”, rt[min] 3.81)

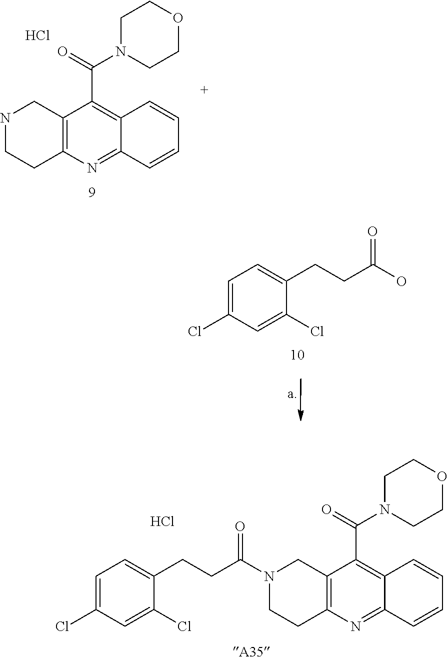

1H-NMR (DMSO-d6): d [ppm] for “A35” 2.85 (m, 2H), 3.00 (m, 2H), 3.16 (m, 3H), 3.41-3.60 (m, 3H) 3.69-4.09 (m, 6H), 4.69-4.95 (m, 2H), 7.34 (m, 1H), 7.44 (m, 1H), 7.53 (m, 1H) 7.97 (t, 1H), 8.07 (d, 1H), 8.15 (t, 1H), 8.28 (d, 1H)*

The following compounds are prepared analogously to Example 4 using the corresponding precursor (10):

### 3-(4-Chloro-2-fluorophenyl)-1-[10-(morpholine-4-carbonyl)-3,4-dihydro-1H-benzo[b][1,6]naphthyridin-2-yl]propan-1-one (“A34”, rt[min] 3.63)

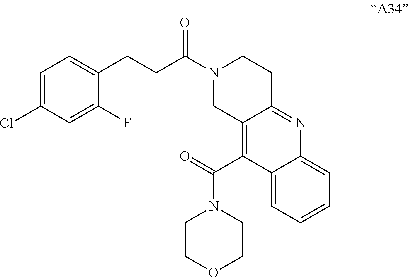

### 1-[10-(Morpholine-4-carbonyl)-3,4-dihydro-1H-benzo[b][1,6]naphthyridin-2-yl]-2-phenylaminoethanone (“A18”, rt[min] 3.04)

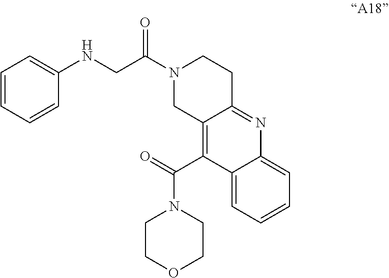

### 4-{2-[10-(Morpholine-4-carbonyl)-3,4-dihydro-1H-benzo[b][1,6]naphthyridin-2-yl]-2-oxoethylamino}benzonitrile (“A19”, rt[min] 3.04)

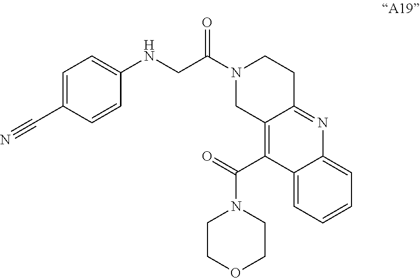

### 3-{2-[10-(Morpholine-4-carbonyl)-3,4-dihydro-1H-benzo[b][1,6]naphthyridin-2-yl]-2-oxoethylamino}benzonitrile (“A20”, rt[min] 3.15)

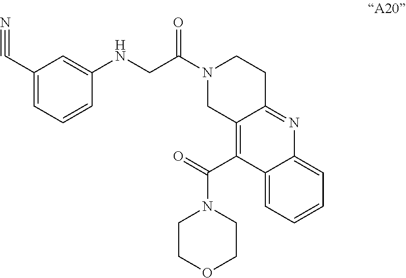

### 3-(2,5-Dimethoxyphenyl)-1-[10-(morpholine-4-carbonyl)-3,4-dihydro-1H-benzo[b][1,6]naphthyridin-2-yl]propan-1-one (“A22”, rt[min] 3.65)

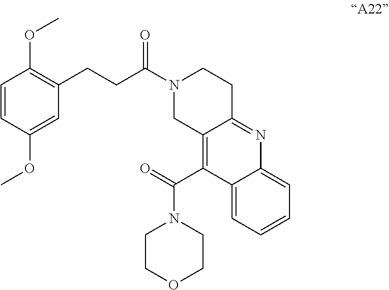

### 3-(2,5-Dimethoxyphenyl)-1-[10-(morpholine-4-carbonyl)-3,4-dihydro-1H-benzo[b][1,6]naphthyridin-2-yl]propan-1-one (“A36”, rt[min] 3.20)

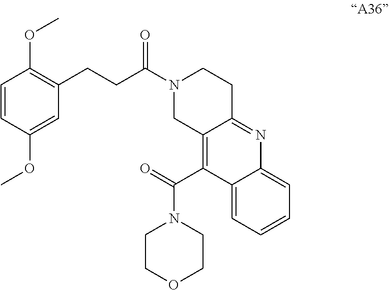

1H-NMR (DMSO-d6): d [ppm] for “A36” 2.69-2.85 (m, 5H), 3.12 (m, 2H), 3.30-3.58 (m, 5H), 3.59-3.77 (m, 6H), 3.77-4.04 (m, 4H), 4.64-4.88 (m, 2H), 6.63-6.90 (m, 3H), 7.93 (t, 1H), 8.02 (d, 1H), 8.12 (t, 1H), 8.23 (d, 1H)*

### 3-(3-Chlorophenyl)-1-[10-(morpholine-4-carbonyl)-3,4-dihydro-1H-benzo-[b][1,6]naphthyridin-2-yl]propan-1-one (“A37”, rt[min] 3.55)

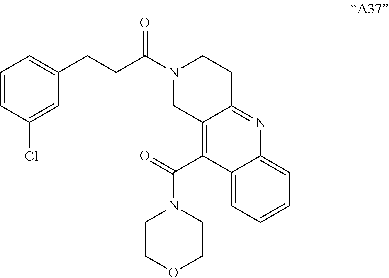

1H-NMR (DMSO-d6): d [ppm] for “A37” 2.83-2.98 (m, 5H), 3.11-3.24 (m, 3H), 3.41-3.60 (m, 2H), 3.72-4.10 (m, 6H), 4.71-4.98 (m, 2H), 7.20-7.38 (m, 4H), 7.96 (t, 1H), 8.08 (d, 1H), 8.16 (t, 1H), 8.31 (d, 1H)*

### 3-(3,4-Dichlorophenyl)-1-[10-(morpholine-4-carbonyl)-3,4-dihydro-1H-benzo[b][1,6]naphthyridin-2-yl]propan-1-one (“A38”, rt[min] 3.71)

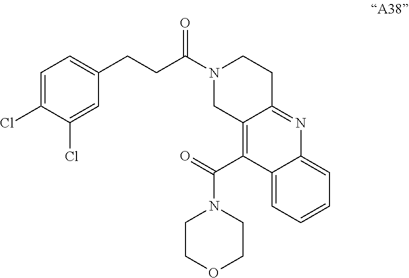

### 1-[10-(Morpholine-4-carbonyl)-3,4-dihydro-1H-benzo[b][1,6]naphthyridin-2-yl]-3-phenylpropan-1-one (“A39”, rt[min] 3.20)

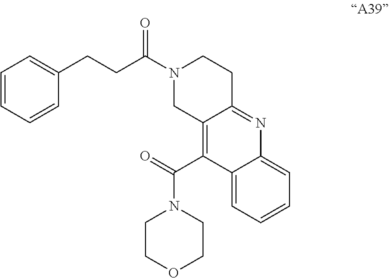

1H-NMR (DMSO-d6): d [ppm] for “A39” 2.76-2.93 (m, 5H), 3.04-3.20 (m, 2H), 3.35-3.58 (m, 3H), 3.63-4.04 (m, 6H), 4.64-4.90 (m, 2H), 7.12-7.30 (m, 5H), 7.93 (t, 1H), 8.03 (d, 1H), 8.12 (t, 1H), 8.30 (d, 1H)*

### N-(5-Chloro-2-methoxyphenyl)-2-[10-(morpholine-4-carbonyl)-3,4-dihydro-1H-benzo[b][1,6]naphthyridin-2-yl]-2-oxoacetamide (“A55”, rt[min] 3.73)

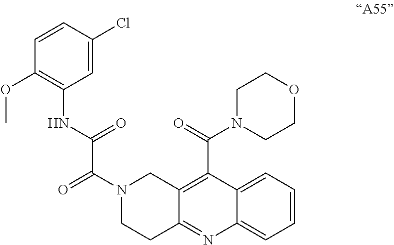

The following compounds can be prepared by the person skilled in the art analogously to the methods described above:

### HPLC Method:

Solvent A: water+0.05% of HCOOH
Solvent B: acetonitrile+0.04% of HCOOH
Flow: 2.0 ml/min
Gradient: 0.0-0.5 min 99% of A
   0.2-3.8 min to 100% of B
   3.8-4.4 min 100% of B
   4.4-5.0 min to 100% of A
Column: Si-ROD® UM9423/100, 3 mm

### 2-{10-[4-(2-Hydroxyethyl)piperazine-1-carbonyl]-3,4-dihydro-1H-benzo-[b][1,6]naphthyridin-2-yl}-N-(1-methyl-1H-pyrazol-4-yl)acetamide (“B1”, rt[min] 2.08)

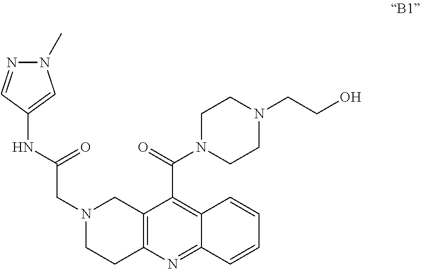

### 2-(4-Chlorophenyl)-2-(2-{10-[4-(2-hydroxyethyl)piperazine-1-carbonyl]-3,4-dihydro-1H-benzo[b][1,6]naphthyridin-2-yl}acetylamino)-N-methylacetamide (“B2”, rt[min] 2.03)

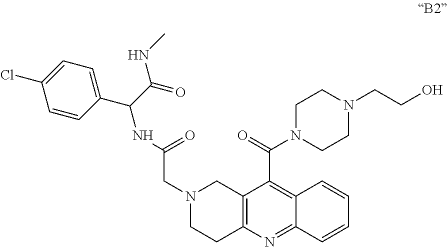

### 2-(2-{10-[4-(2-Hydroxyethyl)piperazine-1-carbonyl]-3,4-dihydro-1H-benzo-[b][1,6]naphthyridin-2-yl}acetylamino)-N-methyl-2-phenylacetamide (“B3”, rt[min] 1.66)

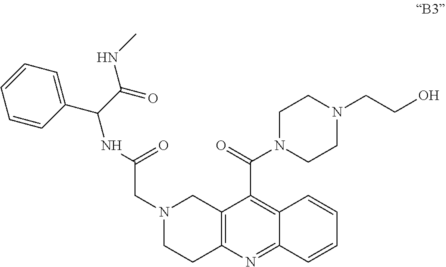

### N-(5-Chloro-2-trifluoromethoxyphenyl)-2-{10-[4-(2-hydroxyethyl)piperazine-1-carbonyl]-3,4-dihydro-1H-benzo[b][1,6]naphthyridin-2-yl}acetamide (“B4”, rt[min] 2.75)

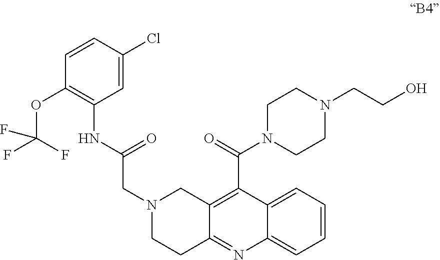

### N-(5-Chloro-2-trifluoromethoxyphenyl)-2-[10-(morpholine-4-carbonyl)-3,4-dihydro-1H-benzo[b][1,6]naphthyridin-2-yl]acetamide (“B5”, rt[min] 3.73)

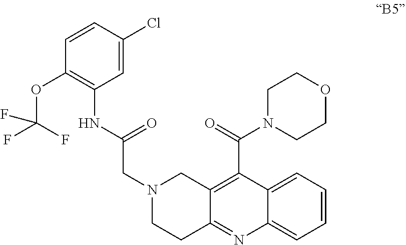

### N-(1H-Benzotriazol-5-yl)-2-[(5-chloro-2-methoxyphenylcarbamoyl)methyl]-1,2,3,4-tetrahydrobenzo[b][1,6]naphthyridine-10-carboxamide (“B22”, rt[min] 4.19)

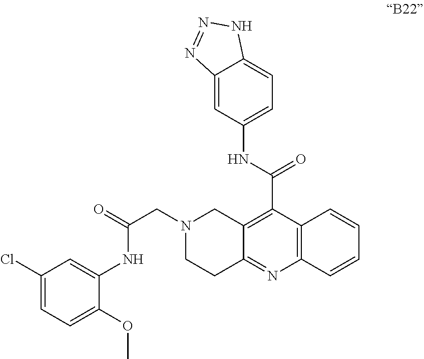

### {2-[2-(3-Chlorophenyl)-2H-pyrazol-3-ylmethyl]-1,2,3,4-tetrahydrobenzo-[b][1,6]naphthyridin-10-yl}morpholin-4-ylmethanone (“B23”, rt[min] 3.28)

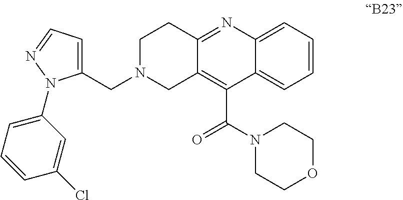

### N-(5-Bromo-3-fluoro-2-methoxyphenyl)-2-[10-(morpholine-4-carbonyl)-3,4-dihydro-1H-benzo[b][1,6]naphthyridin-2-yl]acetamide (“B24”, rt[min] 3.71)

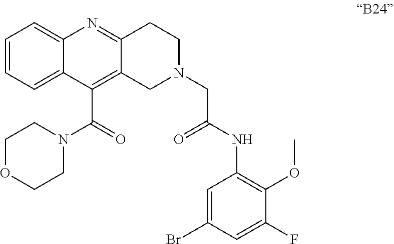

### N-(4′-Amino-5-fluoro-4-methoxybiphenyl-3-yl)-2-[10-(morpholine-4-carbonyl)-3,4-dihydro-1H-benzo[b][1,6]naphthyridin-2-yl]acetamide (“B35”, rt[min] 3.07)

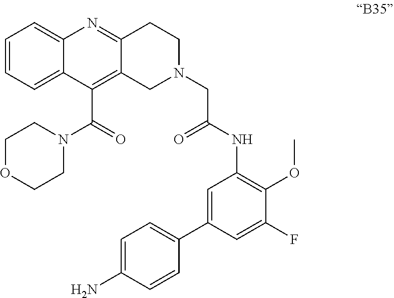

The following HPLC method applies to the following compounds:
Solvent A: water+0.05% of HCOOH
Solvent B: acetonitrile+0.04% of HCOOH
Flow: 2.0 ml/min
Gradient: 0.0-0.5 min 99% of A
   0.2-3.8 min to 100% of B
   3.8-4.4 min 100% of B
   4.4-5.0 min to 100% of A
Column: Si-ROD® UM9423/100, 3 mm

### {2-[2-(1H-Benzoimidazol-2-yl)ethyl]-1,2,3,4-tetrahydrobenzo[b][1,6]naphthyridin-10-yl}-[4-(2-hydroxyethyl)piperazin-1-yl]methanone (“B6”, rt[min] 3.39)

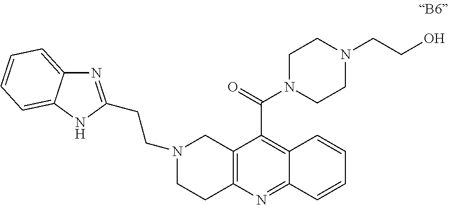

### [2-(1H-Benzoimidazol-2-ylmethyl)-1,2,3,4-tetrahydrobenzo[b][1,6]naphthyridin-10-yl]-[4-(2-hydroxyethyl)piperazin-1-yl]methanone (“B7”, rt[min] 2.08)

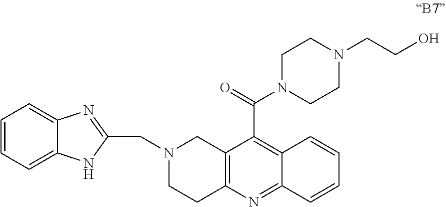

### N-(5-Chlorobenzothiazol-7-yl)-2-{10-[4-(2-hydroxyethyl)piperazine-1-carbonyl]-3,4-dihydro-1H-benzo[b][1,6]naphthyridin-2-yl}acetamide (“B8”, rt[min] 2.20)

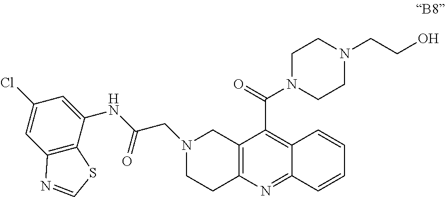

Acetic acid 4-chloro-2-(2-{10-[4-(2-hydroxyethyl)piperazine-1-carbonyl]-3,4-dihydro-1H-benzo[b][1,6]naphthyridin-2-yl}acetylamino)benzyl ester (“B9”, rt[min] 2.33)

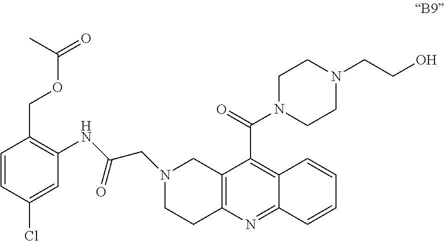

### N-(5-Chloro-2-hydroxymethylphenyl)-2-{10-[4-(2-hydroxyethyl)piperazine-1-carbonyl]-3,4-dihydro-1H-benzo[b][1,6]naphthyridin-2-yl}acetamide (“B10”, rt[min] 2.20)

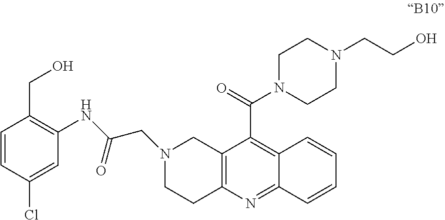

### {2-[5-(5-Chloro-2-methoxyphenyl)-1,2,4-oxadiazol-3-ylmethyl]-1,2,3,4-tetrahydrobenzo[b][1,6]naphthyridin-10-yl}-[4-(2-hydroxyethyl)piperazin-1-yl]-methanone (“B11”, rt[min] 2.30)

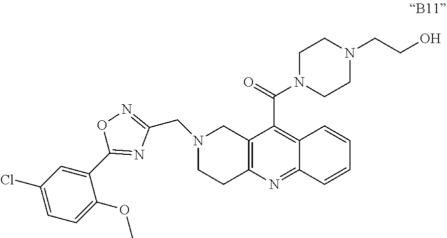

### 2-{10-[4-(2-Hydroxyethyl)piperazine-1-carbonyl]-3,4-dihydro-1H-benzo-[b][1,6]naphthyridin-2-yl}-N-phenylacetamide (“B12”, rt[min] 2.04)

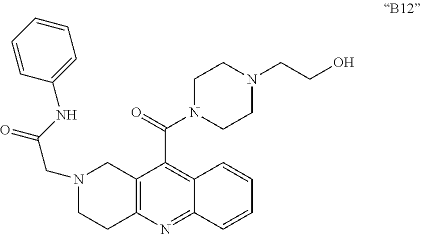

### 2-{10-[4-(2-Hydroxyethyl)piperazine-1-carbonyl]-3,4-dihydro-1H-benzo-[b][1,6]naphthyridin-2-yl}-N-(2-trifluoromethylsulfanylphenyl)acetamide (“B13”, rt[min] 2.36)

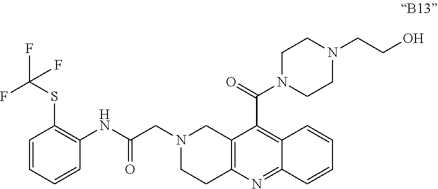

### {2-[2-(5-Chloro-2-methoxyphenylamino)ethyl]-1,2,3,4-tetrahydrobenzo-[b][1,6]naphthyridin-10-yl}-[4-(2-hydroxyethyl)piperazin-1-yl]methanone (“B14”, rt[min] 2.21)

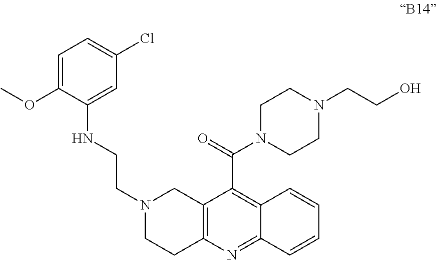

### [2-(6-Chloro-1H-benzoimidazol-2-ylmethyl)-1,2,3,4-tetrahydrobenzo-[b][1,6]naphthyridin-10-yl]-[4-(2-hydroxyethyl)piperazin-1-yl]methanone (“B15”, rt[min] 2.12)

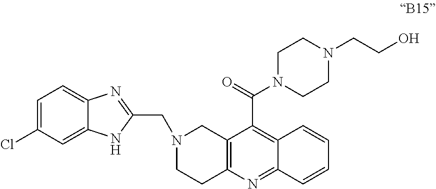

### 1-(4-Chlorobenzyl)-3-{2-[10-(morpholine-4-carbonyl)-3,4-dihydro-1H-benzo-[b][1,6]naphthyridin-2-yl]ethyl}urea (“B16”, rt[min] 2.37)

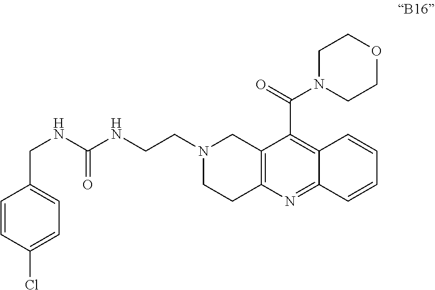

### N-(5-Chloro-2-methylsulfanylphenyl)-2-{10-[4-(2-hydroxyethyl)piperazine-1-carbonyl]-3,4-dihydro-1H-benzo[b][1,6]naphthyridin-2-yl}acetamide (“B17”, rt[min]2.42)

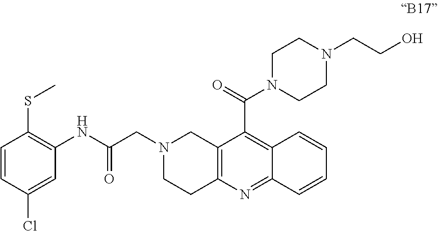

### [4-(2-Hydroxyethyl)piperazin-1-yl]-[2-(5-methylamino-1,3,4-oxadiazol-2-yl-methyl)-1,2,3,4-tetrahydrobenzo[b][1,6]naphthyridin-10-yl]methanone (“B18”, rt[min] 2.03)

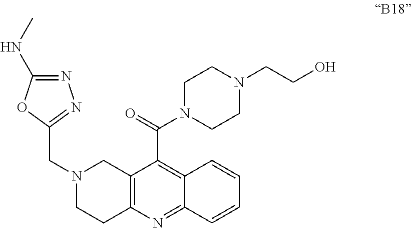

### N-(1-Methylpiperidin-4-yl)-2-(4-chloro-3H-imidazo[4,5-c]pyridin-2-ylmethyl)-1,2,3,4-tetrahydrobenzo[b][1,6]naphthyridine-10-carboxamide (“B19”, rt[min] 2.02)

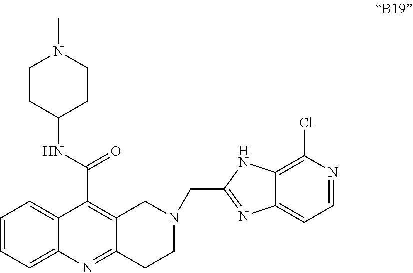

### N-(1-Methylpiperidin-4-yl)-2-[5-(5-chloro-2-methoxyphenyl)-1,2,4-oxadiazol-3-ylmethyl]-1,2,3,4-tetrahydrobenzo[b][1,6]naphthyridine-10-carboxamide (“B20”, rt[min] 2.35)

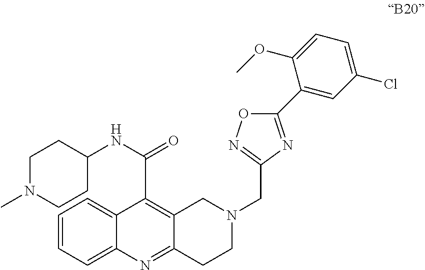

### N-Biphenyl-3-yl-2-{10-[4-(2-hydroxyethyl)piperazine-1-carbonyl]-3,4-dihydro-1H-benzo[b][1,6]naphthyridin-2-yl}acetamide (“B21”, rt[min] 2.37)

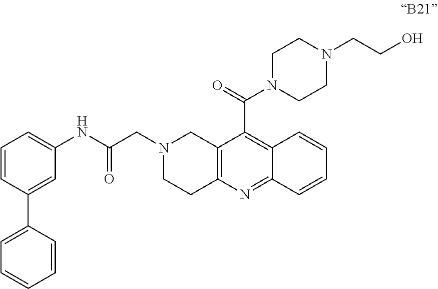

### N-(1-Methylpiperidin-4-yl)-2-[2-(5-fluoro-2-methoxyphenyl)-1H-imidazol-4-yl-methyl]-1,2,3,4-tetrahydrobenzo[b][1,6]naphthyridine-10-carboxamide (“B25”, rt[min] 2.05)

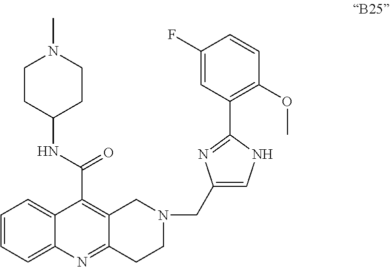

### N-(1-Methylpiperidin-4-yl)-2-(5-chlorobenzothiazol-2-ylmethyl)-1,2,3,4-tetrahydrobenzo[b][1,6]naphthyridine-10-carboxamide (“B26”, rt[min] 2.35)

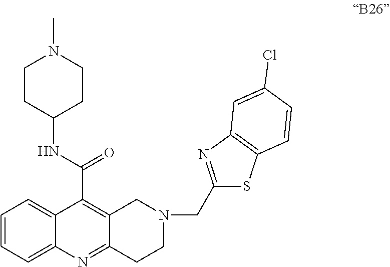

### N-(1-Methylpiperidin-4-yl)-2-[3-(5-chloro-2-methoxyphenyl)-1,2,4-oxadiazol-5-ylmethyl]-1,2,3,4-tetrahydrobenzo[b][1,6]naphthyridine-10-carboxamide (“B27”, rt[min] 2.35)

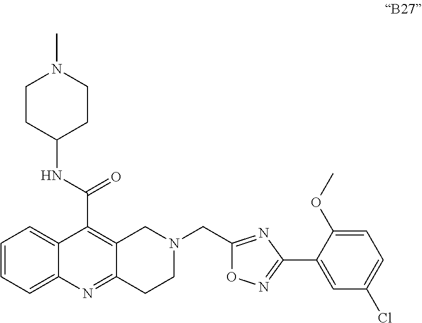

### N-(1-Methylpiperidin-4-yl)-2-(7-chloro-1H-benzoimidazol-2-ylmethyl)-1,2,3,4-tetrahydrobenzo[b][1,6]naphthyridine-10-carboxamide (“B28”, rt[min] 2.11)

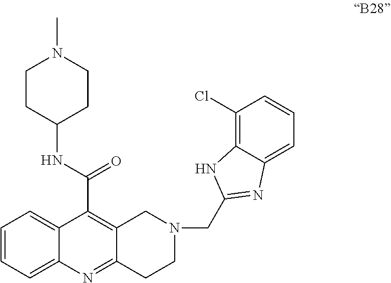

### N-(2-Dimethylaminoethyl)-2-[5-(5-chloro-2-methoxyphenyl)-1,3,4-oxadiazol-2-ylmethyl]-1,2,3,4-tetrahydrobenzo[b][1,6]naphthyridine-10-carboxamide (“B29”, rt[min] 2.33)

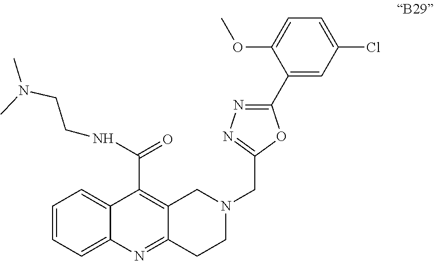

### [5-(5-Chloro-2-methoxyphenyl)-1,3,4-oxadiazol-2-ylmethyl]-[2-({2-[5-(5-chloro-2-methoxyphenyl)-1,3,4-oxadiazol-2-ylmethyl]-1,2,3,4-tetrahydrobenzo[b][1,6]naphthyridine-10-carbonyl}amino)ethyl]dimethylammonium (“B30”, rt[min] 2.57)

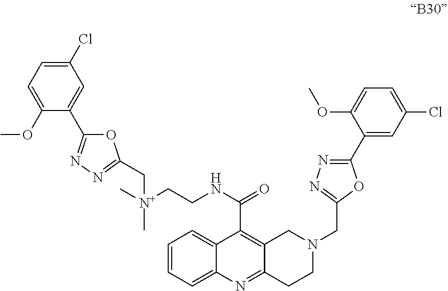

### N-(2-Methoxyethyl)-2-[5-(5-chloro-2-methoxyphenyl)-1,3,4-oxadiazol-2-yl-methyl]-1,2,3,4-tetrahydrobenzo[b][1,6]naphthyridine-10-carboxamide (“B31”, rt[min] 2.68)

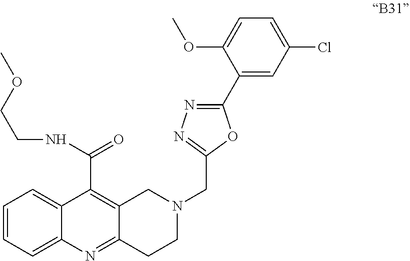

### N-Ethyl-2-[5-(5-chloro-2-methoxyphenyl)-1,3,4-oxadiazol-2-ylmethyl]-1,2,3,4-tetrahydrobenzo[b][1,6]naphthyridine-10-carboxamide (“B32”, rt[min] 2.71)

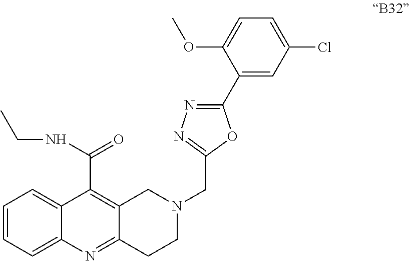

### N-(1-Methylpiperidin-4-yl)-2-[5-(5-chloro-2-methoxyphenyl)-1,3,4-oxadiazol-2-ylmethyl]-1,2,3,4-tetrahydrobenzo[b][1,6]naphthyridine-10-carboxamide (“B33”, rt[min] 2.31)

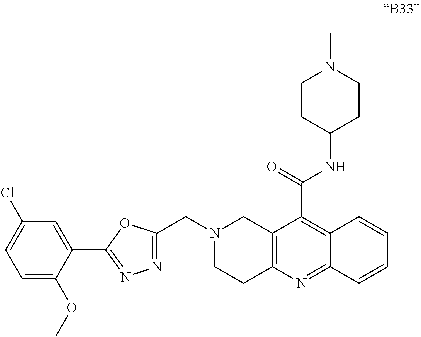

### N-(1-Methylpiperidin-4-yl)-2-{2-[N′-(5-chloro-2-methoxybenzoyl)hydrazino]-2-oxoethyl}-1,2,3,4-tetrahydrobenzo[b][1,6]naphthyridine-10-carboxamide (“B34”, rt[min] 2.10)

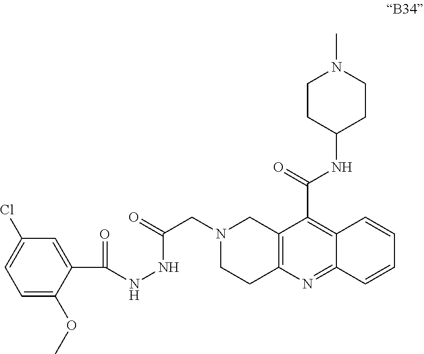

### (2-{2-[5-(5-Chloro-2-methoxyphenyl)-1,3,4-oxadiazol-2-yl]ethyl}-1,2,3,4-tetrahydrobenzo[b][1,6]naphthyridin-10-yl)morpholin-4-ylmethanone (“B36”, rt[min] 2.51)

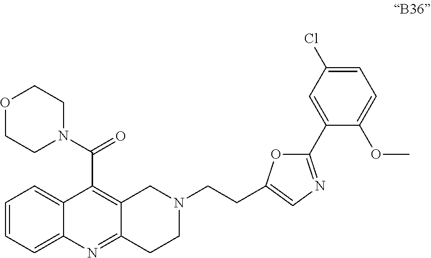

### N-(1-Methylpiperidin-4-yl)-2-[2-(2-amino-5-chlorophenylcarbamoylethyl]-1,2,3,4-tetrahydrobenzo[b][1,6]naphthyridine-10-carboxamide (“B37”, rt[min] 2.03)

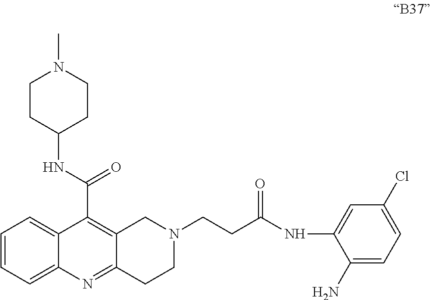

### N-(1-Methylpiperidin-4-yl)-2-[3-(5-chloro-2-methoxyphenyl)propionyl]-1,2,3,4-tetrahydrobenzo[b][1,6]naphthyridine-10-carboxamide (“B38”, rt[min] 2.42)

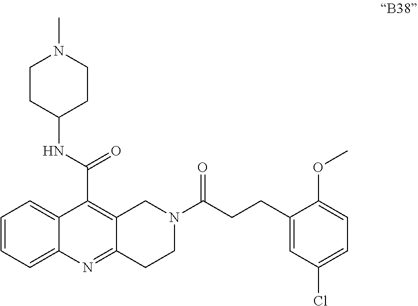

### 2-[5-(5-Chloro-2-methoxyphenyl)-1,3,4-oxadiazol-2-ylmethyl]-1,2,3,4-tetrahydrobenzo[b][1,6]naphthyridine-10-carboxamide (“B39”, rt[min] 2.55)

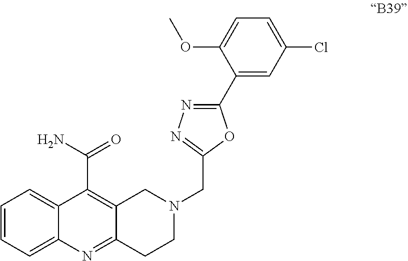

### N-(1-Methylpiperidin-4-yl)-2-[(E)-3-(5-chloro-2-methoxyphenyl)acryloyl]-1,2,3,4-tetrahydrobenzo[b][1,6]naphthyridine-10-carboxamide (“B40”, rt[min] 2.51)

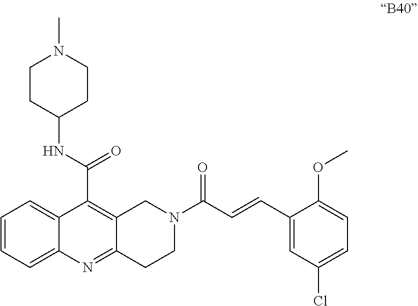

### 5-Chloro-2-methoxybenzyl 10-(1-methylpiperidin-4-ylcarbamoyl)-3,4-dihydro-1H-benzo[b][1,6]naphthyridine-2-carboxylate (“B41”, rt[min] 2.44)

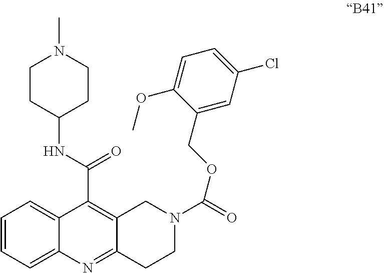

### N-(2-Fluoroethyl)-2-[(5-chloro-2-methoxyphenylcarbamoyl)methyl]-1,2,3,4-tetrahydrobenzo[b][1,6]naphthyridine-10-carboxamide (“B42”, rt[min] 2.74)

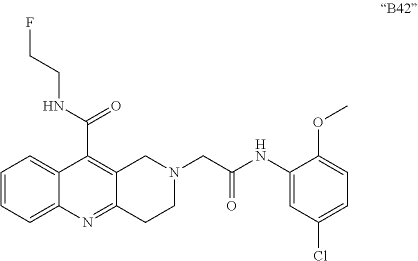

### N-(Tetrahydropyran-4-yl)-2-[(5-chloro-2-methoxyphenylcarbamoyl)methyl]-1,2,3,4-tetrahydrobenzo[b][1,6]naphthyridine-10-carboxamide (“B43, rt[min] 2.68)

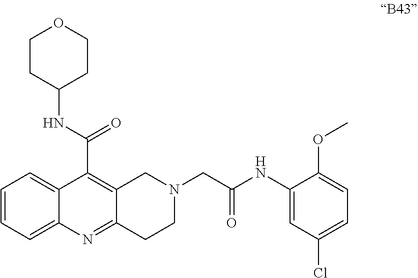

### N-(1-Methylpiperidin-4-yl)-2-[1-(5-chloro-2-methoxyphenyl)-2-oxopyrrolidin-3-ylmethyl]-1,2,3,4-tetrahydrobenzo[b][1,6]naphthyridine-10-carboxamide (“B44”, rt[min] 2.14)

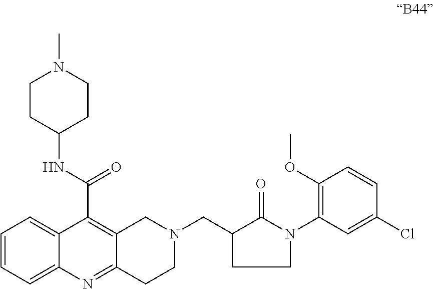

### N-(5-Chloro-2-methoxyphenyl)-2-[10-(4-dimethylaminopiperidine-1-carbonyl)-3,4-dihydro-1H-benzo[b][1,6]naphthyridin-2-yl]acetamide (“B45”, rt[min] 2.42)

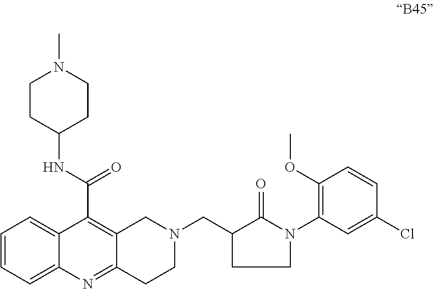

### N-(1-Methylpiperidin-4-yl)-2-((S)-7-chloro-2,3-dihydrobenzo-1,4-dioxin-2-yl-methyl)-1,2,3,4-tetrahydrobenzo[b][1,6]naphthyridine-10-carboxamide (“B46”, rt[min] 2.23)

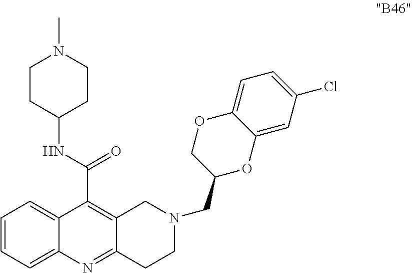

### N-(2-Oxopiperidin-3-yl)-2-[(5-chloro-2-methoxyphenylcarbamoyl)methyl]-1,2,3,4-tetrahydrobenzo[b][1,6]naphthyridine-10-carboxamide (“B47”, rt[min] 2.58)

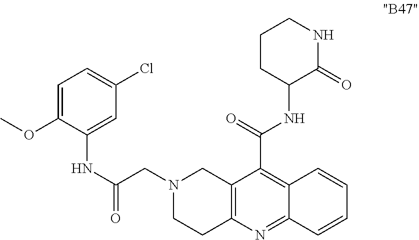

### N-(1-Methylpiperidin-4-yl)-2-(6-chlorochroman-3-ylmethyl)-1,2,3,4-tetrahydrobenzo[b][1,6]naphthyridine-10-carboxamide (“B48” rt[min] 2.23)

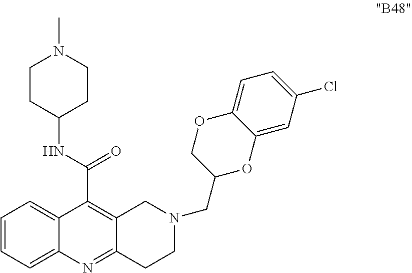

### N-(1-Methylpiperidin-4-yl)-2-(6-chloro-2H-chromen-3-ylmethyl)-1,2,3,4-tetrahydrobenzo[b][1,6]naphthyridine-10-carboxamide (“B49”, rt[min] 2.23)

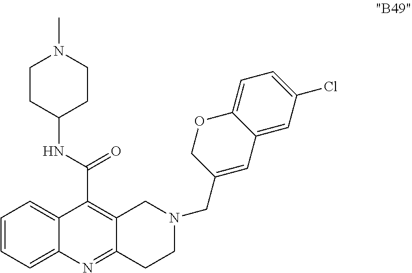

### N-(1-Methylpiperidin-4-yl)-2-(6,8-dichloro-2-oxo-2H-chromene-3-carbonyl)-1,2,3,4-tetrahydrobenzo[b][1,6]naphthyridine-10-carboxamide (“B50”, rt[min] 2.49)

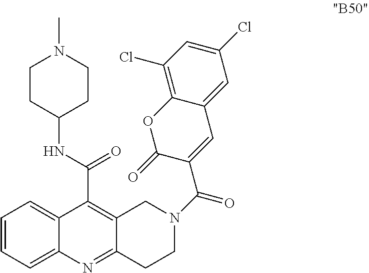

### 1-(5-Chloro-2-hydroxyphenyl)-2-[10-(morpholine-4-carbonyl)-3,4-dihydro-1H-benzo[b][1,6]naphthyridin-2-yl]ethanone (“B51”, rt[min] 2.50)

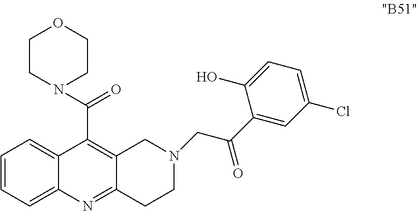

### [2-(2-Chloropyridin-4-ylmethyl)-1,2,3,4-tetrahydrobenzo[b][1,6]naphthyridin-10-yl]morpholin-4-ylmethanone (“B52”, rt[min] 1.87)

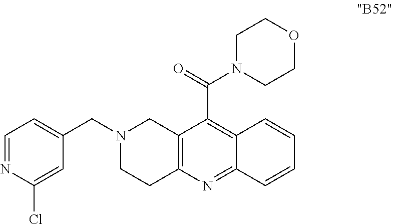

### N-Piperidin-3-yl-2-[(5-chloro-2-methoxyphenylcarbamoyl)methyl]-1,2,3,4-tetrahydrobenzo[b][1,6]naphthyridine-10-carboxamide (“B53”, rt[min] 2.35)

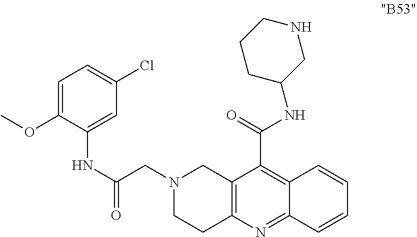

### N-(1-Methylpiperidin-4-yl)-2-[2-(5-bromo-2-methoxyphenyl)thiazol-4-yl-methyl]-1,2,3,4-tetrahydrobenzo[b][1,6]naphthyridine-10-carboxamide (“B54”, rt[min] 2.31)

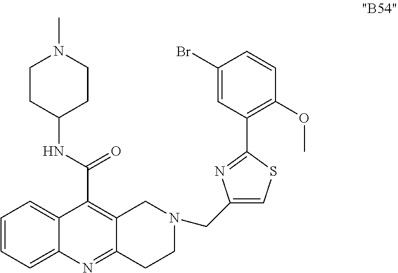

### N-(1-Methylpiperidin-4-yl)-2-[2-(5-bromo-2-methoxyphenyl)thiazol-4-yl-methyl]-1,2,3,4-tetrahydrobenzo[b][1,6]naphthyridine-10-carboxamide (“B55”, rt[min] 2.56)

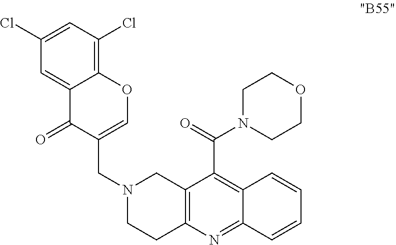

### {2-[2-(5-Chloro-2-methoxyphenyl)pyridin-4-ylmethyl]-1,2,3,4-tetrahydrobenzo[b][1,6]naphthyridin-10-yl}morpholin-4-ylmethanone (“B56”, rt[min] 2.60)

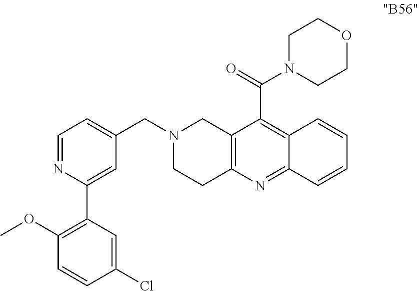

### N-(1-Methylpiperidin-4-yl)-2-(5,7-dichloro-8-hydroxyquinolin-2-ylmethyl)-1,2,3,4-tetrahydrobenzo[b][1,6]naphthyridine-10-carboxamide (“B57”, rt[min] 2.45)

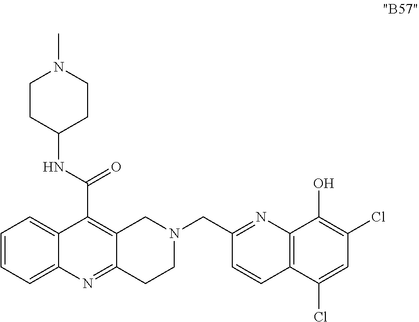

### Pharmacological Data

### Autotaxin Inhibition (Enzyme Test)

TABLE 1

| Compound | |
|---|---|
| No. | IC50 |
| “A1” | A |
| “A7” | A |
| “A13” | A |
| “A23” | A |
| “A24” | A |
| “A29” | A |
| “A66” | A |
| “A67” | A |
| “A68” | A |
| “A69” | A |
| “A12” | B |
| “A14” | B |
| “A15” | B |
| “A16” | B |
| “A17” | B |
| “A25” | B |
| “A26” | B |
| “A21” | B |
| “A27” | B |
| “A28” | B |
| “A30” | B |
| “A40” | B |
| “A42” | B |
| “A43” | B |
| “A48” | B |
| “A59” | B |
| “A53” | B |
| “A54” | B |
| “A61” | B |
| “A75” | B |
| “A78” | B |
| “A80” | B |
| “A87” | B |
| “A9” | C |
| “A10” | C |
| “A11” | C |
| “A33” | C |
| “A31” | C |
| “A41” | C |
| “A45” | C |
| “A52” | C |
| “A58” | C |
| “A60” | C |
| “A64” | C |
| “A65” | C |
| “A71” | C |
| “A73” | C |
| “A74” | C |
| “A76” | C |
| “A79” | C |

TABLE 2

| Compound | |
|---|---|
| No. | IC50 |
| “B2” | C |
| “B4” | C |
| “B5” | C |
| “B14” | C |
| “B17” | C |
| “B20” | C |
| “B21” | C |
| “B22” | B |
| “B24” | B |
| “B29” | C |
| “B30” | C |
| “B31” | C |
| “B32” | C |
| “B33” | C |
| “B35” | C |
| “B37” | C |
| “B40” | C |
| “B42” | C |
| “B43” | B |
| “B45” | B |
| “B47” | B |
| “B52” | C |
| “B53” | C |
| “B57” | C |

| | |
|---|---|
| IC50: | <100 nM = A |
| | 100 nM-1 μM = B |
| | >1 μM = C |

### EXAMPLE A

### Autotaxin Test (Enzyme Test)

### Test Description

The autotaxin activity is measured indirectly using Amplex Red reagent. Amplex Red is measured here as fluorogenic indicator for the H₂O₂ formed. In detail, autotaxin converts the substrate lysophosphatidylcholine (LPC) into phosphocholine and lysophosphatidylic acid (LPA). After this reaction, the phosphocholine is reacted with alkaline phosphatase to give inorganic phosphate and choline. In the next step, choline is oxidised by choline oxidase to give betaine, with formation of H₂O₂. H₂O₂ reacts with Amplex Red reagent in the presence of peroxidase (horseradish peroxidase) in a 1:1 stoichiometry and forms the highly fluorescent resorufin. The fluorescence is measured in a reaction-dependent kinetic mode in order that fluorescent signals from possible other fluorescent substances which are not involved in the reaction can be corrected out.

### Test Procedure

1.5 μl of a standard solution or of the test substances (substances with the name A(n))in individual concentrations dissolved in 20 mM Hepes pH 7.2 with a maximum of 7.7% of DMSO are pre-incubated together with 10 μl (16 ng) of highly purified recombinant autotaxin in a black microtitre plate provided with 384 wells at 22° C. for 30 min. The reaction is then initiated by addition of 5 μl of L-α-lysophosphatidylcholine (LPC), where the final concentration of LPC is 75 μM. The mixture is incubated at 37° C. for 90 min. After the incubation, Amplex Red reagent, peroxidase (horseradish peroxidase) and choline oxidase is added, and the fluorescence is immediately measured at 612 nm with excitation of 485 nm in a “Tecan Ultra multimode” reader. The activity of autotaxin is calculated indirectly via detection of the H₂O₂ formed.

### Material:

Microtitre plate: PS microplate, 384 wells, small volume, black Corning, Cat#3677
Protein: recombinant autotaxin (Baculovirale Hi5 Expression)
Substrate: L-α-lysophosphatidylcholine (chicken egg)); Avanti Polar Lipids # 830071 P
Standard: C14 LPA, Avanti Polar Lipids, Cat# 857120P
Detection reagent: Amplex Red reagent; Invitrogen # A12222; dissolved in 1.923 ml of DMSO peroxidase type VI-A (horseradish) from Sigma # P6782; dissolved in 7.45 ml of test buffer, choline oxidase; Sigma # C5896; dissolved in 2.47 ml of test buffer
Detection reagent mix: 1:100 dilution of Amplex Red reagent in test buffer
Test buffer: 200 mM Tris HCl, Merck, Cat # 1.08219, pH 7.9, 0.1% of BSA, lipid-free, Roche Cat#775835

The following examples relate to medicaments:

### EXAMPLE B

### Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### EXAMPLE C

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### EXAMPLE D

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE E

### Ointment

500 mg of an active ingredient of the formula I are mixed with 99.5 g of Vaseline under aseptic conditions.

### EXAMPLE F

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE G

### Dragees

Tablets are pressed analogously to Example E and subsequently coated in a conventional manner with a coating of sucrose, potato starch, talc, tragacanth and dye.

### EXAMPLE H

### Capsules

2 kg of active ingredient of the formula I are introduced into hard gelatine capsules in a conventional manner in such a way that each capsule contains 20 mg of the active ingredient.

### EXAMPLE I

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 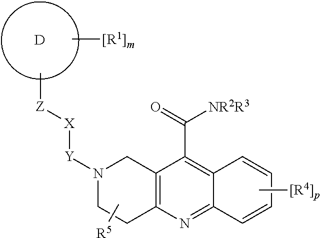
in which
D denotes Ar or Het¹,
Het¹ denotes a mono- or bicyclic saturated, unsaturated or aromatic heterocycle having 1 to 4 N, O and/or S atoms, which is unsubstituted or may be mono-, di or trisubstituted by Hal, A, OA, Ar, OH and or ═O,
R¹ in each case, independently of one another, denotes H, Hal, OA, OH, A, phenyl or CN may be mono- or polysubstituted,
Het² denotes a monocyclic, saturated heterocycle having 1-3N and/or O atoms, which is unsubstituted or may be mono- or disubstituted by ═O,
R⁴ in each case, independently of one another, denotes H, Hal, OA, OH, A, may be mono- or polysubstituted,
X, Y each, independently of one another, are absent, or denote —CH₂—, —(CH₂)₂—, —CO— or —CHOH—, where only one of the radicals X or Y may be absent,
R², R³ each, independently of one another, denote R; R² and R³ together also denote an alkyl chain having 2-6C atoms, in which, in addition, one CH₂ group may be replaced by O, NH or NA', A′ denotes alkyl having 1-6C atoms, or CH₂CH₂OH, COO(CH₂)ₙAr, (CH₂)ₙAr, (CH₂)ₙHet², (CH₂)ₙNA₂ or Cyc,
R⁵ denotes H, Hal, NH₂, OH, OA or A, may be,
R denotes H, A, Cyc, (CH₂)ₙAr or (CH₂)ₙHet mono- or polysubstituted,
Z denotes O, NH, —CH(CONHA)NH—, CH₂NHCONH, —CH═CH— or is absent,
Cyc denotes cyclic alkyl having 3-7C atoms,
A denotes linear or branched alkyl having 1-10C atoms, in which 1-7H atoms may be replaced by OR, CN, NR₂, F and/or Cl and/or in which one or two non-adjacent CH₂ groups may be replaced by O, NH, S, SO, SO₂ and/or by CH═CH groups,
or
cyclic alkyl having 3-7C atoms,
Ar denotes phenyl, indanyl, naphthyl or biphenyl, each of which is un-substituted or mono-, di-, tri-, tetra- or pentasubstituted by Hal, A, (CR₂)ₙOR, O(CR₂)ₙAr², (CR₂)ₙNR₂, SR, NO₂, CN, COOR, CONR₂, NRCOA, NRSO₂A, SO₂NR₂, S(O)ₘA, CO-Het, (CR₂)ₙHet, O(CR₂)ₙ—NR₂, O(CR₂)ₙHet, NHCOOA, NHCONR₂, NHCOO(CR₂)ₙNR₂, NHCOO(CR₂)ₙHet, CR═CRAr², SO₂Het, NHCONH(CR₂)ₙNR₂, NHCONH(CR₂)ₙHet, OCONH(CR₂)ₙNR₂, CONH(CR₂)ₙHet, CONR(CR₂)ₙNR₂, CONR(CR₂)ₙHet and/or COA,
Het denotes a mono-, bi- or tricyclic saturated, unsaturated or aromatic heterocycle having 1 to 4 N, O and/or S atoms, which may be unsubstituted or mono-, di- or trisubstituted by Hal, A, Ar², O(CR₂)ₙAr², (CR₂)ₙOR, (CR₂)ₙNR₂, SR, NO₂, CN, COOR, CONR₂, NRCOA, NRSO₂A, SO₂NR₂, S(O)_{q}A, CO-Het², (CR₂)ₙHet², O(CR₂)ₙNR₂, O(CR₂)ₙHet², NHCOOA, NHCONR₂, NHCOO(CR₂)ₙNR₂, NHCOO(CR₂)ₙHet², NHCONH(CR₂)ₙNR₂, NHCONH(CR₂)ₙHet², OCONH(CR₂)ₙNR₂, OCONH(CR₂)ₙHet², CO-Het², CHO, COA, ═S, ═NH, ═NA and/or ═O,
Hal denotes F, Cl, Br or I,
n denotes 0, 1 or 2,
m denotes 0, 1, 2, 3, 4, or 5
p denotes 1, 2, 3, or 4
and pharmaceutically usable salts and stereoisomers thereof, including mixtures thereof in all ratios.

2. Compounds according to claim 1 in which
denotes H, Hal, CN, phenyl, OA or OH,
and pharmaceutically usable salts and stereoisomers thereof, including mixtures thereof in all ratios.

3. Compounds according to claim 1 in which
R⁴ denotes H, Hal, A, OA or OH,
and pharmaceutically usable derivatives, solvates, tautomers, salts and stereoisomers thereof, including mixtures thereof in all ratios.

4. Compounds according to claim 1 in which
R⁵H
and pharmaceutically usable salts and stereoisomers thereof, including mixtures thereof in all ratios.

5. Compounds according to claim 1 in which
R², R³ together denotes morpholinyl, piperazinyl, 1-methylpiperazinyl, 1-ethyl-4-methylpiperazinyl, 2-(4-methylpiperazin-1-yl)ethyl, 1-methyl-4-propylpiperazinyl, 1-cyclopentyl-4-methylpiperazinyl, 1-benzyl-4-methyl-1,4-diazepanyl or 1-benzyl-4-methylpiperazinyl,
and pharmaceutically usable salts and stereoisomers thereof, including mixtures thereof in all ratios.

6. Compounds according to claim 1 in which
Het¹ particularly preferably denotes piperazyl, morpholinyl, piperidinyl, pyrrolidinyl, furanyl, thienyl, pyrrolyl, imidazolyl, pyrazolyl, oxazolyl, isoxazolyl, thiazolyl, isothiazolyl, pyridyl, pyrimidinyl, triazolyl, benzotriazylyl, benzofuranyl, 2,3-dihydrobenzoxazolyl, benzoxazolyl, dihydrobenzofuranyl or tetrazolyl, each of which is unsubstituted or mono-, di- or trisubstituted by A and/or (CH₂)ₙAr.

7. Compounds according to claim 1 in which
Het² particularly preferably denotes pyrrolidinyl, morpholinyl, piperidinyl or piperazinyl, each of which is unsubstituted or mono- or di substituted by Hal, OH, OA, A and/or ═O.

8. Compounds according to claim 1 in which
R¹ denotes H, Hal, CN, phenyl, OA or OH;
R⁴ denotes H, Hal, A, OA or OH;
R⁵ denotes H and
R², R³ together denote morpholinyl, piperazinyl, 1-methylpiperazinyl, 1-ethyl-4-methylpiperazinyl, 2-(4-methylpiperazin-1-yl)ethyl, 1-methyl-4-propylpiperazinyl, 1-cyclopentyl-4-methylpiperazinyl, 1-benzyl-4-methyl-1,4-diazepanyl or 1-benzyl-4-methylpiperazinyl,
Het¹ particularly preferably denotes piperazyl, morpholinyl, piperidinyl, pyrrolidinyl, furanyl, thienyl, pyrrolyl, imidazolyl, pyrazolyl, oxazolyl, isoxazolyl, thiazolyl, isothiazolyl, pyridyl, pyrimidinyl, triazolyl, benzotriazylyl, benzofuranyl, 2,3-dihydrobenzoxazolyl, benzoxazolyl, dihydrobenzofuranyl or tetrazolyl, each of which is unsubstituted or mono-, di- or trisubstituted by A and/or (CH₂)ₙAr,
Het² particularly preferably denotes pyrrolidinyl, morpholinyl, piperidinyl or piperazinyl, each of which is unsubstituted or mono- or disubstituted by Hal, OH, OA, A and/or ═O,
and pharmaceutically usable salts and stereoisomers thereof, including mixtures thereof in all ratios.

9. Compounds according to claim 1 selected from the group

| No. | Name and/or structure |
|---|---|
| “A1” | N-(5-Chloro-2-methoxyphenyl)-2-[10-(4-methyl- |
| | piperazine-1-carbonyl)-3,4-dihydro-1H-benzo |
| | [b][1,6]naphthyridin-2-yl]acetamide |
| “A2” | (2-Benzyl-1,2,3,4-tetrahydrobenzo[b][1,6]naphthyridin- |
| | 10-yl)-(4-methylpiperazin-1-yl)methanone |
| “A3” | 1-[10-(4-Methylpiperazine-1-carbonyl)-3,4-dihydro-1H- |
| | benzo[b][1,6]naphthyridin-2-yl]-2-phenylaminoethanone |
| “A4” | 2-(2-Chloro-5-methoxyphenylamino)-1-[10-(4-methyl- |
| | piperazine-1-carbonyl)-3,4-dihydro-1H-benzo- |
| | [b][1,6]naphthyridin-2-yl]ethanone |
| “A5” | 4-{2-[10-(4-Methylpiperazine-1-carbonyl)-3,4-dihydro- |
| | 1H-benzo[b][1,6]naphthyridin-2-yl]-2-oxoethylamino}- |
| | benzonitrile |
| “A6” | 3-{2-[10-(4-Methylpiperazine-1-carbonyl)-3,4-dihydro- |
| | 1H-benzo[b][1,6]naphthyridin-2-yl]-2-oxoethylamino}- |
| | benzonitrile |
| “A7” | N-(5-Chloro-2-methoxyphenyl)-2-[10-(morpholine-4- |
| | carbonyl)-3,4-dihydro-1H-benzo[b][1,6]naphthyridin-2- |
| | yl]acetamide |
| “A8” | (2-Benzyl-1,2,3,4-tetrahydrobenzo[b][1,6]naphthyridin- |
| | 10-yl)morpholin-4-ylmethanone |
| “A9” | N-(3-Chlorophenyl)-2-[10-(morpholine-4-carbonyl)-3,4- |
| | dihydro-1H-benzo[b][1,6]naphthyridin-2-yl]acetamide |
| “A10” | N-(2-Methoxyphenyl)-2-[10-(morpholine-4-carbonyl)- |
| | 3,4-dihydro-1H-benzo[b][1,6]naphthyridin-2-yl]- |
| | acetamide |
| “A11” | N-(5-Chloro-2-methylphenyl)-2-[10-(morpholine-4- |
| | carbonyl)-3,4-dihydro-1H-benzo[b][1,6]naphthyridin-2- |
| | yl]acetamide |
| “A12” | N-(2-Methoxy-5-methylphenyl)-2-[10-(morpholine-4- |
| | carbonyl)-3,4-dihydro-1H-benzo[b][1,6]naphthyridin-2- |
| | yl]acetamide |
| “A13” | N-(5-Bromo-2-methoxyphenyl)-2-[10-(morpholine-4- |
| | carbonyl)-3,4-dihydro-1H-benzo[b][1,6]naphthyridin-2- |
| | yl]acetamide |
| “A14” | N-(4-Methoxybiphenyl-3-yl)-2-[10-(morpholine-4- |
| | carbonyl)-3,4-dihydro-1H-benzo[b][1,6]naphthyridin-2- |
| | yl]acetamide |
| “A15” | -(5-Chloro-2-methoxyphenyl)-2-[7-chloro-10- |
| | (morpholine-4-carbonyl)-3,4-dihydro-1H- |
| | benzo[b][1,6]naphthyridin-2-yl]acetamide |
| “A16” | N-(5-Chloro-2-methoxyphenyl)-2-[6-chloro-10- |
| | (morpholine-4-carbonyl)-3,4-dihydro-1H- |
| | benzo[b][1,6]naphthyridin-2-yl]acetamide |
| “A17” | N-(5-Chloro-2-methoxyphenyl)-2-[7-ethyl-10- |
| | (morpholine-4-carbonyl)-3,4-dihydro-1H- |
| | benzo[b][1,6]naphthyridin-2-yl]acetamide |
| “A18” | 1-[10-(Morpholine-4-carbonyl)-3,4-dihydro-1H- |
| | benzo[b][1,6]naphthyridin-2-yl]-2-phenylaminoethanone |
| “A19” | 4-{2-[10-(Morpholine-4-carbonyl)-3,4-dihydro-1H- |
| | benzo[b][1,6]naphthyridin-2-yl]-2-oxoethylamino}- |
| | benzonitrile |
| “A20” | 3-{2-[10-(Morpholine-4-carbonyl)-3,4-dihydro-1H- |
| | benzo[b][1,6]naphthyridin-2-yl]-2-oxoethylamino}- |
| | benzonitrile |
| “A21” | 3-{2-[10-(Morpholine-4-carbonyl)-3,4-dihydro-1H- |
| | benzo[b][1,6]naphthyridin-2-yl]-2-oxoethylamino}- |
| | benzonitrile |
| “A22” | 3-(2,5-Dimethoxyphenyl)-1-[10-(morpholine-4-carbonyl)- |
| | 3,4-dihydro-1H-benzo[b][1,6]naphthyridin-2-yl]propan-1- |
| | one |
| “A23” | N-(5-Chloro-2-methoxyphenyl)-2-[10-(4-ethylpiperazine- |
| | 1-carbonyl)-3,4-dihydro-1H-benzo[b][1,6]naphthyridin-2- |
| | yl]acetamide |
| “A24” | N-(5-Chloro-2-methoxyphenyl)-2-[10-(4-ethylpiperazine- |
| | 1-carbonyl)-3,4-dihydro-1H-benzo[b][1,6]naphthyridin-2- |
| | yl]acetamide |
| “A25” | Benzyl 4-{2-[(5-chloro-2-methoxyphenylcarbamoyl)- |
| | methyl]-1,2,3,4-tetrahydrobenzo[b][1,6]naphthyridine-10- |
| | carbonyl}piperazine-1-carboxylate |
| “A26” | 2-[10-(4-Benzyl-1,4-diazepan-1-carbonyl)-3,4-dihydro- |
| | 1H-benzo[b][1,6]naphthyridin-2-yl]-N-(5-chloro-2- |
| | methoxyphenyl)acetamide |
| “A27” | N-(5-Chloro-2-methoxyphenyl)-2-[10-(piperazine-1- |
| | carbonyl)-3,4-dihydro-1H-benzo[b][1,6]naphthyridin-2- |
| | yl]acetamide |
| “A28” | N-(5-Chloro-2-methoxyphenyl)-2-[10-(4-cyclopentyl- |
| | piperazine-1-carbonyl)-3,4-dihydro-1H- |
| | benzo[b][1,6]naphthyridin-2-yl]acetamide |
| “A29” | N-(5-Chloro-2-methoxyphenyl)-2-[10-(4-propyl- |
| | piperazine-1-carbonyl)-3,4-dihydro-1H- |
| | benzo[b][1,6]naphthyridin-2-yl]acetamide |
| “A30” | 2-[10-(4-Benzylpiperazine-1-carbonyl)-3,4-dihydro-1H- |
| | benzo[b][1,6]naphthyridin-2-yl]-N-(5-chloro-2- |
| | methoxyphenyl)acetamide |
| “A31” | 3-(2,4-Dichlorophenyl)-1-[10-(4-methylpiperazine-1- |
| | carbonyl)-3,4-dihydro-1H-benzo[b][1,6]naphthyridin-2- |
| | yl]propan-1-one |
| “A32” | 3-(2,5-Dimethoxyphenyl)-1-[10-(4-methylpiperazine-1- |
| | carbonyl)-3,4-dihydro-1H-benzo[b][1,6]naphthyridin-2- |
| | yl]propan-1-one |
| “A33” | 3-(4-Chloro-2-fluorophenyl)-1-[10-(4-methylpiperazine- |
| | 1-carbonyl)-3,4-dihydro-1H-benzo[b][1,6]naphthyridin-2- |
| | yl]propan-1-one |
| “A34” | 3-(4-Chloro-2-fluorophenyl)-1-[10-(morpholine-4- |
| | carbonyl)-3,4-dihydro-1H-benzo[b][1,6]naphthyridin-2- |
| | yl]propan-1-one |
| “A35” | 3-(2,4-Dichlorophenyl)-1-[10-(morpholine-4-carbonyl)- |
| | 3,4-dihydro-1H-benzo[b][1,6]naphthyridin-2-yl]propan-1- |
| | one |
| “A36” | 3-(2,5-Dimethoxyphenyl)-1-[10-(morpholine-4-carbonyl)- |
| | 3,4-dihydro-1H-benzo[b][1,6]naphthyridin-2-yl]propan-1- |
| | one |
| “A37” | 3-(3-Chlorophenyl)-1-[10-(morpholine-4-carbonyl)-3,4- |
| | dihydro-1H-benzo[b][1,6]naphthyridin-2-yl]propan-1-one |
| “A38” | 3-(3,4-Dichlorophenyl)-1-[10-(morpholine-4-carbonyl)- |
| | 3,4-dihydro-1H-benzo[b][1,6]naphthyridin-2-yl]propan-1- |
| | one |
| “A39” | 1-[10-(Morpholine-4-carbonyl)-3,4-dihydro-1H-benzo- |
| | [b][1,6]naphthyridin-2-yl]-3-phenylpropan-1-one |
| “A40” | 2-[6-Bromo-10-(morpholine-4-carbonyl)-3,4-dihydro-1H- |
| | benzo[b][1,6]naphthyridin-2-yl]-N-(5-chloro-2- |
| | methoxyphenyl)acetamide |
| “A41” | N-(5-Chloro-2-ethoxyphenyl)-2-[10-(morpholine-4- |
| | carbonyl)-3,4-dihydro-1H-benzo[b][1,6]naphthyridin-2- |
| | yl]acetamide |
| “A42” | 2-[7-Bromo-10-(morpholine-4-carbonyl)-3,4-dihydro-1H- |
| | benzo[b][1,6]naphthyridin-2-yl]-N-(5-chloro-2- |
| | methoxyphenyl)acetamide |
| “A43” | N-(5-Bromobenzofuran-7-yl)-2-[10-(morpholine-4- |
| | carbonyl)-3,4-dihydro-1H-benzo[b][1,6]naphthyridin-2- |
| | yl]acetamide |
| “A44” | 2-[10-(Morpholine-4-carbonyl)-3,4-dihydro-1H- |
| | benzo[b][1,6]naphthyridin-2-yl]-N-(2-trifluoromethoxy- |
| | 5-trifluoromethylphenyl)acetamide |
| “A45” | N-Benzofuran-7-yl-2-[10-(morpholine-4-carbonyl)-3,4- |
| | dihydro-1H-benzo[b][1,6]naphthyridin-2-yl]acetamide |
| “A46” | N-(2,3-Dihydrobenzofuran-7-yl)-2-[10-(morpholine-4- |
| | carbonyl)-3,4-dihydro-1H-benzo[b][1,6]naphthyridin-2- |
| | yl]acetamide |
| “A47” | N-(4-Chloropyridin-2-yl)-2-[10-(morpholine-4-carbonyl)- |
| | 3,4-dihydro-1H-benzo[b][1,6]naphthyridin-2-yl]- |
| | acetamide |
| “A48” | N-(5-Chlorobenzofuran-7-yl)-2-[10-(morpholine-4- |
| | carbonyl)-3,4-dihydro-1H-benzo[b][1,6]naphthyridin-2- |
| | yl]acetamide |
| “A49” | N-(5-Chloro-2-isopropoxyphenyl)-2-[10-(morpholine-4- |
| | carbonyl)-3,4-dihydro-1H-benzo[b][1,6]naphthyridin-2- |
| | yl]acetamide |
| “A50” | N-(5-Chloropyridin-3-yl)-2-[10-(morpholine-4-carbonyl)- |
| | 3,4-dihydro-1H-benzo[b][1,6]naphthyridin-2-yl]- |
| | acetamide |
| “A51” | N-(5-Chloro-2-methoxypyridin-3-yl)-2-[10-(morpholine- |
| | 4-carbonyl)-3,4-dihydro-1H-benzo[b][1,6]naphthyridin-2- |
| | yl]acetamide |
| “A52” | N-(5-Chloro-2-ethoxypyridin-3-yl)-2-[10-(morpholine-4- |
| | carbonyl)-3,4-dihydro-1H-benzo[b][1,6]naphthyridin-2- |
| | yl]acetamide |
| “A53” | N-(4-Chloro-2-hydroxyphenyl)-2-[10-(morpholine-4- |
| | carbonyl)-3,4-dihydro-1H-benzo[b][1,6]naphthyridin-2- |
| | yl]acetamide |
| “A54” | N-[5-Chloro-2-(2-hydroxyethoxy)phenyl]-2-[10- |
| | (morpholine-4-carbonyl)-3,4-dihydro-1H- |
| | benzo[b][1,6]naphthyridin-2-yl]acetamide |
| “A55” | N-(5-Chloro-2-methoxyphenyl)-2-[10-(morpholine-4- |
| | carbonyl)-3,4-dihydro-1H-benzo[b][1,6]naphthyridin-2- |
| | yl]-2-oxoacetamide |
| “A56” | N-(5-Chloro-2-methoxyphenyl)-2-{10-[4-(2- |
| | hydroxyethyl)piperazine-1-carbonyl]-3,4-dihydro-1H- |
| | benzo[b][1,6]naphthyridin-2-yl}-2-oxoacetamide |
| “A57” | {2-[(5-Chlorobenzofuran-7-ylamino)methyl]-1,2,3,4- |
| | tetrahydrobenzo[b][1,6]naphthyridin-10-yl}-[4-(2- |
| | hydroxyethyl)piperazin-1-yl]methanone |
| “A58” | 4-Chloro-2-({10-[4-(2-hydroxyethyl)piperazine-1- |
| | carbonyl]-3,4-dihydro-1H-benzo[b][1,6]naphthyridin-2- |
| | ylmethyl}amino)benzonitrile |
| “A59” | Methyl 4-chloro-2-({10-[4-(2-hydroxyethyl)piperazine-1- |
| | carbonyl]-3,4-dihydro-1H-benzo[b][1,6]naphthyridin-2- |
| | ylmethyl}amino)benzoate |
| “A60” | N-(5-Chloro-2-isopropoxyphenyl)-2-{10-[4-(2- |
| | hydroxyethyl)piperazine-1-carbonyl]-3,4-dihydro-1H- |
| | benzo[b][1,6]naphthyridin-2-yl}acetamide |
| “A61” | N-[5-Chloro-2-(2-hydroxyethoxy)phenyl]-2-{10-[4-(2- |
| | hydroxyethyl)piperazine-1-carbonyl]-3,4-dihydro-1H- |
| | benzo[b][1,6]naphthyridin-2-yl}acetamide |
| “A62” | N-(5-Fluoro-2-methoxyphenyl)-2-{10-[4-(2-hydroxy- |
| | ethyl)piperazine-1-carbonyl]-3,4-dihydro-1H-benzo- |
| | [b][1,6]naphthyridin-2-yl}acetamide |
| “A63” | 4-Chloro-2-(2-{10-[4-(2-hydroxyethyl)piperazine-1- |
| | carbonyl]-3,4-dihydro-1H-benzo[b][1,6]naphthyridin-2- |
| | yl}acetylamino)benzoic acid |
| “A64” | N-[3-Chloro-4-(2-oxopyrrolidin-1-yl)phenyl]-2-{10-[4- |
| | (2-hydroxyethyl)piperazine-1-carbonyl]-3,4-dihydro-1H- |
| | benzo[b][1,6]naphthyridin-2-yl}acetamide |
| “A65” | N-[3-Chloro-4-(3-oxomorpholin-4-yl)phenyl]-2-{10-[4- |
| | (2-hydroxyethyl)piperazine-1-carbonyl]-3,4-dihydro-1H- |
| | benzo[b][1,6]naphthyridin-2-yl}acetamide |
| “A66” | N-(5-Chloro-2-methoxyphenyl)-2-{10-[4-(2-piperidin-1- |
| | ylethyl)piperazine-1-carbonyl]-3,4-dihydro-1H- |
| | benzo[b][1,6]naphthyridin-2-yl}acetamide |
| “A67” | N-(5-Chloro-2-methoxyphenyl)-2-{10-[4-(2-dimethyl- |
| | aminoethyl)piperazine-1-carbonyl]-3,4-dihydro-1H- |
| | benzo[b][1,6]naphthyridin-2-yl}acetamide |
| “A68” | N-(5-Chloro-2-methoxyphenyl)-2-{10-[4-(2-morpholin-4- |
| | ylethyl)piperazine-1-carbonyl]-3,4-dihydro-1H- |
| | benzo[b][1,6]naphthyridin-2-yl}acetamide |
| “A69” | N-(5-Bromo-2-methoxyphenyl)-2-{10-[4-(2-hydroxy- |
| | ethyl)piperazine-1-carbonyl]-3,4-dihydro-1H- |
| | benzo[b][1,6]naphthyridin-2-yl}acetamide |
| “A70” | 1-(6-Chloro-2,3-dihydrobenzo-1,4-oxazin-4-yl)-2-{10-[4- |
| | (2-hydroxyethyl)piperazine-1-carbonyl]-3,4-dihydro-1H- |
| | benzo[b][1,6]naphthyridin-2-yl}ethanone |
| “A71” | N-(5-Chloro-2,4-dimethoxyphenyl)-2-{10-[4-(2-hydroxy- |
| | ethyl)piperazine-1-carbonyl]-3,4-dihydro-1H- |
| | benzo[b][1,6]naphthyridin-2-yl}acetamide |
| “A72” | N-(3-Chloro-4-methoxyphenyl)-2-{10-[4-(2-hydroxy- |
| | ethyl)piperazine-1-carbonyl]-3,4-dihydro-1H- |
| | benzo[b][1,6]naphthyridin-2-yl}acetamide |
| “A73” | N-(3-Chloro-4-methylphenyl)-2-{10-[4-(2-hydroxyethyl)- |
| | piperazine-1-carbonyl]-3,4-dihydro-1H- |
| | benzo[b][1,6]naphthyridin-2-yl}acetamide |
| “A74” | N-(3-Chloro-4-fluorophenyl)-2-{10-[4-(2-hydroxyethyl)- |
| | piperazine-1-carbonyl]-3,4-dihydro-1H- |
| | benzo[b][1,6]naphthyridin-2-yl}acetamide |
| “A75” | N-(2,5-Dichlorophenyl)-2-{10-[4-(2-hydroxyethyl)- |
| | piperazine-1-carbonyl]-3,4-dihydro-1H- |
| | benzo[b][1,6]naphthyridin-2-yl}acetamide |
| “A76” | N-(3,4-Dichlorophenyl)-2-{10-[4-(2-hydroxyethyl)- |
| | piperazine-1-carbonyl]-3,4-dihydro-1H- |
| | benzo[b][1,6]naphthyridin-2-yl}acetamide |
| “A77” | N-(3-Chloro-2-fluorophenyl)-2-{10-[4-(2-hydroxyethyl)- |
| | piperazine-1-carbonyl]-3,4-dihydro-1H- |
| | benzo[b][1,6]naphthyridin-2-yl}acetamide |
| “A78” | N-(5-Chloro-2-fluorophenyl)-2-{10-[4-(2-hydroxyethyl)- |
| | piperazine-1-carbonyl]-3,4-dihydro-1H- |
| | benzo[b][1,6]naphthyridin-2-yl}acetamide |
| “A79” | N-(5-Chlorobenzooxazol-7-yl)-2-{10-[4-(2-hydroxy- |
| | ethyl)piperazine-1-carbonyl]-3,4-dihydro-1H-benzo- |
| | [b][1,6]naphthyridin-2-yl}acetamide |
| “A80” | N-(3,5-Dichloro-2-methoxyphenyl)-2-{10-[4-(2-hydroxy- |
| | ethyl)piperazine-1-carbonyl]-3,4-dihydro-1H- |
| | benzo[b][1,6]naphthyridin-2-yl}acetamide |
| “A81” | N-(5-Chloro-2-oxo-2,3-dihydrobenzooxazol-7-yl)-2-{10- |
| | [4-(2-hydroxyethyl)piperazine-1-carbonyl]-3,4-dihydro- |
| | 1H-benzo[b][1,6]naphthyridin-2-yl}acetamide |
| “A82” | N-(6-Chloro-3H-benzotriazol-4-yl)-2-{10-[4-(2- |
| | hydroxyethyl)piperazine-1-carbonyl]-3,4-dihydro-1H- |
| | benzo[b][1,6]naphthyridin-2-yl}acetamide |
| “A83” | 1-(6-Chloro-2,3-dihydroindol-1-yl)-2-[10-(morpholine-4- |
| | carbonyl)-3,4-dihydro-1H-benzo[b][1,6]naphthyridin-2- |
| | yl]ethanone |
| “A84” | 1-(3,4-Dihydro-2H-quinolin-1-yl)-2-[10-(morpholine-4- |
| | carbonyl)-3,4-dihydro-1H-benzo[b][1,6]naphthyridin-2- |
| | yl]ethanone |
| “A85” | 1-(2,3-Dihydrobenzo-1,4-oxazin-4-yl)-2-[10- |
| | (morpholine-4-carbonyl)-3,4-dihydro-1H- |
| | benzo[b][1,6]naphthyridin-2-yl]ethanone |
| “A86” | N-(5-Chloro-2-methoxyphenyl)-N-methyl-2-[10- |
| | (morpholine-4-carbonyl)-3,4-dihydro-1H- |
| | benzo[b][1,6]naphthyridin-2-yl]acetamide |
| “A87” | N-(5-Chloro-2-methoxyphenyl)-N-methyl-2-[10- |
| | (morpholine-4-carbonyl)-3,4-dihydro-1H- |
| | benzo[b][1,6]naphthyridin-2-yl]acetamide |
| “B1” | 2-(4-Chlorophenyl)-2-(2-{10-[4-(2-hydroxyethyl)- |
| | piperazine-1-carbonyl]-3,4-dihydro-1H- |
| | benzo[b][1,6]naphthyridin-2-yl}acetylamino)-N- |
| | methylacetamide |
| “B2” | 2-(4-Chlorophenyl)-2-(2-{10-[4-(2-hydroxyethyl)- |
| | piperazine-1-carbonyl]-3,4-dihydro-1H- |
| | benzo[b][1,6]naphthyridin-2-yl}acetylamino)-N- |
| | methylacetamide |
| “B3” | 2-(2-{10-[4-(2-Hydroxyethyl)piperazine-1-carbonyl]-3,4- |
| | dihydro-1H-benzo[b][1,6]naphthyridin-2-yl}- |
| | acetylamino)-N-methyl-2-phenylacetamide |
| “B4” | N-(5-Chloro-2-trifluoromethoxyphenyl)-2-{10-[4-(2- |
| | hydroxyethyl)piperazine-1-carbonyl]-3,4-dihydro-1H- |
| | benzo[b][1,6]naphthyridin-2-yl}acetamide |
| “B5” | N-(5-Chloro-2-trifluoromethoxyphenyl)-2-[10- |
| | (morpholine-4-carbonyl)-3,4-dihydro-1H- |
| | benzo[b][1,6]naphthyridin-2-yl]acetamide |
| “B6” | {2-[2-(1H-Benzoimidazol-2-yl)ethyl]-1,2,3,4-tetra- |
| | hydrobenzo[b][1,6]naphthyridin-10-yl}-[4-(2-hydroxy- |
| | ethyl)piperazin-1-yl]methanone |
| “B7” | [2-(1H-Benzoimidazol-2-ylmethyl)-1,2,3,4-tetra- |
| | hydrobenzo[b][1,6]naphthyridin-10-yl]-[4-(2-hydroxy- |
| | ethyl)piperazin-1-yl]methanone |
| “B8” | N-(5-Chlorobenzothiazol-7-yl)-2-{10-[4-(2-hydroxy- |
| | ethyl)piperazine-1-carbonyl]-3,4-dihydro-1H- |
| | benzo[b][1,6]naphthyridin-2-yl}acetamide |
| “B9” | Acetic acid 4-chloro-2-(2-{10-[4-(2-hydroxyethyl)- |
| | piperazine-1-carbonyl]-3,4-dihydro-1H- |
| | benzo[b][1,6]naphthyridin-2-yl}acetylamino)benzyl ester |
| “B10 | N-(5-Chloro-2-hydroxymethylphenyl)-2-{10-[4-(2- |
| | hydroxyethyl)piperazine-1-carbonyl]-3,4-dihydro-1H- |
| | benzo[b][1,6]naphthyridin-2-yl}acetamide |
| “B11” | {2-[5-(5-Chloro-2-methoxyphenyl)-1,2,4-oxadiazol-3-yl- |
| | methyl]-1,2,3,4-tetrahydrobenzo[b][1,6]naphthyridin-10- |
| | yl}-[4-(2-hydroxyethyl)piperazin-1-yl]methanone |
| “B12” | 2-{10-[4-(2-Hydroxyethyl)piperazine-1-carbonyl]-3,4- |
| | dihydro-1H-benzo[b][1,6]naphthyridin-2-yl}-N- |
| | phenylacetamide |
| “B13” | 2-{10-[4-(2-Hydroxyethyl)piperazine-1-carbonyl]-3,4- |
| | dihydro-1H-benzo[b][1,6]naphthyridin-2-yl}-N-(2- |
| | trifluoromethylsulfanylphenyl)acetamide |
| “B14” | {2-[2-(5-Chloro-2-methoxyphenylamino)ethyl]-1,2,3,4- |
| | tetrahydrobenzo[b][1,6]naphthyridin-10-yl}-[4-(2- |
| | hydroxyethyl)piperazin-1-yl]methanone |
| “B15” | [2-(6-Chloro-1H-benzoimidazol-2-ylmethyl)-1,2,3,4- |
| | tetrahydrobenzo[b][1,6]naphthyridin-10-yl]-[4-(2- |
| | hydroxyethyl)piperazin-1-yl]methanone |
| “B16” | 1-(4-Chlorobenzyl)-3-{2-[10-(morpholine-4-carbonyl)- |
| | 3,4-dihydro-1H-benzo[b][1,6]naphthyridin-2-yl]ethyl}- |
| | urea |
| “B17” | N-(5-Chloro-2-methylsulfanylphenyl)-2-{10-[4-(2- |
| | hydroxyethyl)piperazine-1-carbonyl]-3,4-dihydro-1H- |
| | benzo[b][1,6]naphthyridin-2-yl}acetamide |
| “B18” | [4-(2-Hydroxyethyl)piperazin-1-yl]-[2-(5-methylamino- |
| | 1,3,4-oxadiazol-2-ylmethyl)-1,2,3,4- |
| | tetrahydrobenzo[b][1,6]naphthyridin-10-yl]methanone |
| “B19” | N-(1-Methylpiperidin-4-yl)-2-(4-chloro-3H-imidazo[4,5- |
| | c]pyridin-2-ylmethyl)-1,2,3,4-tetrahydrobenzo- |
| | [b][1,6]naphthyridine-10-carboxamide |
| “B20 | N-(1-Methylpiperidin-4-yl)-2-[5-(5-chloro-2-methoxy- |
| | phenyl)-1,2,4-oxadiazol-3-ylmethyl]-1,2,3,4-tetra- |
| | hydrobenzo[b][1,6]naphthyridine-10-carboxamide |
| “B21” | N-Biphenyl-3-yl-2-{10-[4-(2-hydroxyethyl)piperazine-1- |
| | carbonyl]-3,4-dihydro-1H-benzo[b][1,6]naphthyridin-2- |
| | yl}acetamide |
| “B22” | N-(1H-Benzotriazol-5-yl)-2-[(5-chloro-2-methoxy- |
| | phenylcarbamoyl)methyl]-1,2,3,4-tetrahydrobenzo- |
| | [b][1,6]naphthyridine-10-carboxamide |
| “B23” | {2-[2-(3-Chlorophenyl)-2H-pyrazol-3-ylmethyl]-1,2,3,4- |
| | tetrahydrobenzo[b][1,6]naphthyridin-10-yl}morpholin-4- |
| | ylmethanone |
| “B24” | N-(5-Bromo-3-fluoro-2-methoxyphenyl)-2-[10- |
| | (morpholine-4-carbonyl)-3,4-dihydro-1H- |
| | benzo[b][1,6]naphthyridin-2-yl]acetamide |
| “B25” | N-(1-Methylpiperidin-4-yl)-2-[2-(5-fluoro-2-methoxy- |
| | phenyl)-1H-imidazol-4-ylmethyl]-1,2,3,4-tetrahydro- |
| | benzo[b][1,6]naphthyridine-10-carboxamide |
| “B26” | N-(1-Methylpiperidin-4-yl)-2-(5-chlorobenzothiazol-2- |
| | ylmethyl)-1,2,3,4-tetrahydrobenzo[b][1,6]naphthyridine- |
| | 10-carboxamide |
| “B27” | N-(1-Methylpiperidin-4-yl)-2-[3-(5-chloro-2-methoxy- |
| | phenyl)-1,2,4-oxadiazol-5-ylmethyl]-1,2,3,4-tetra- |
| | hydrobenzo[b][1,6]naphthyridine-10-carboxamide |
| “B28” | N-(1-Methylpiperidin-4-yl)-2-(7-chloro-1H-benzo- |
| | imidazol-2-ylmethyl)-1,2,3,4-tetrahydro- |
| | benzo[b][1,6]naphthyridine-10-carboxamide |
| “B29” | N-(2-Dimethylaminoethyl)-2-[5-(5-chloro-2-methoxy- |
| | phenyl)-1,3,4-oxadiazol-2-ylmethyl]-1,2,3,4-tetrahydro- |
| | benzo[b][1,6]naphthyridine-10-carboxamide |
| “B30 | [5-(5-Chloro-2-methoxyphenyl)-1,3,4-oxadiazol-2- |
| | ylmethyl]-[2-({2-[5-(5-chloro-2-methoxyphenyl)-1,3,4- |
| | oxadiazol-2-ylmethyl]-1,2,3,4-tetrahydro- |
| | benzo[b][1,6]naphthyridine-10-carbonyl}amino)ethyl]- |
| | dimethylammonium |
| “B31” | N-(2-Methoxyethyl)-2-[5-(5-chloro-2-methoxyphenyl)- |
| | 1,3,4-oxadiazol-2-ylmethyl]-1,2,3,4-tetrahydrobenzo- |
| | [b][1,6]naphthyridine-10-carboxamide |
| “B32” | N-Ethyl-2-[5-(5-chloro-2-methoxyphenyl)-1,3,4- |
| | oxadiazol-2-ylmethyl]-1,2,3,4-tetrahydro- |
| | benzo[b][1,6]naphthyridine-10-carboxamide |
| “B33” | N-(1-Methylpiperidin-4-yl)-2-[5-(5-chloro-2- |
| | methoxyphenyl)-1,3,4-oxadiazol-2-ylmethyl]-1,2,3,4- |
| | tetrahydrobenzo[b][1,6]naphthyridine-10-carboxamide |
| “B34” | N-(1-Methylpiperidin-4-yl)-2-{2-[N′-(5-chloro-2- |
| | methoxybenzoyl)hydrazino]-2-oxoethyl}-1,2,3,4-tetra- |
| | hydrobenzo[b][1,6]naphthyridine-10-carboxamide |
| “B35” | N-(4′-Amino-5-fluoro-4-methoxybiphenyl-3-yl)-2-[10- |
| | (morpholine-4-carbonyl)-3,4-dihydro-1H- |
| | benzo[b][1,6]naphthyridin-2-yl]acetamide |
| “B36” | 2-{2-[5-(5-Chloro-2-methoxyphenyl)-1,3,4-oxadiazol-2- |
| | yl]ethyl}-1,2,3,4-tetrahydrobenzo[b][1,6]naphthyridin-10- |
| | yl)morpholin-4-ylmethanone |
| “B37” | N-(1-Methylpiperidin-4-yl)-2-[2-(2-amino-5-chloro- |
| | phenylcarbamoyl)ethyl]-1,2,3,4-tetrahydrobenzo[b][1,6]- |
| | naphthyridine-10-carboxamide |
| “B38” | N-(1-Methylpiperidin-4-yl)-2-[3-(5-chloro-2-methoxy- |
| | phenyl)propionyl]-1,2,3,4-tetrahydrobenzo[b][1,6]- |
| | naphthyridine-10-carboxamide |
| “B39” | 2-[5-(5-Chloro-2-methoxyphenyl)-1,3,4-oxadiazol-2- |
| | ylmethyl]-1,2,3,4-tetrahydrobenzo[b][1,6]naphthyridine- |
| | 10-carboxamide |
| “B40 | N-(1-Methylpiperidin-4-yl)-2-[(E)-3-(5-chloro-2- |
| | methoxyphenyl)acryloyl]-1,2,3,4-tetrahydro- |
| | benzo[b][1,6]naphthyridine-10-carboxamide |
| “B41” | 5-Chloro-2-methoxybenzyl 10-(1-methylpiperidin-4-yl- |
| | carbamoyl)-3,4-dihydro-1H-benzo[b][1,6]naphthyridine- |
| | 2-carboxylate |
| “B42” | N-(2-Fluoroethyl)-2-[(5-chloro-2-methoxyphenyl- |
| | carbamoyl)methyl]-1,2,3,4-tetrahydrobenzo[b][1,6]- |
| | naphthyridine-10-carboxamide |
| “B43” | N-(Tetrahydropyran-4-yl)-2-[(5-chloro-2-methoxy- |
| | phenylcarbamoyl)methyl]-1,2,3,4-tetrahydrobenzo- |
| | [b][1,6]naphthyridine-10-carboxamide |
| “B44” | N-(1-Methylpiperidin-4-yl)-2-[1-(5-chloro-2-methoxy- |
| | phenyl)-2-oxopyrrolidin-3-ylmethyl]-1,2,3,4-tetra- |
| | hydrobenzo[b][1,6]naphthyridine-10-carboxamide |
| “B45” | N-(5-Chloro-2-methoxyphenyl)-2-[10-(4-dimethyl- |
| | aminopiperidine-1-carbonyl)-3,4-dihydro-1H-benzo- |
| | [b][1,6]naphthyridin-2-yl]acetamide |
| “B46” | N-(1-Methylpiperidin-4-yl)-2-((S)-7-chloro-2,3- |
| | dihydrobenzo-1,4-dioxin-2-ylmethyl)-1,2,3,4-tetrahydro- |
| | benzo[b][1,6]naphthyridine-10-carboxamide |
| “B47” | N-(2-Oxopiperidin-3-yl)-2-[(5-chloro-2-methoxy- |
| | phenylcarbamoyl)methyl]-1,2,3,4-tetrahydrobenzo- |
| | [b][1,6]naphthyridine-10-carboxamide |
| “B48” | N-(1-Methylpiperidin-4-yl)-2-(6-chlorochroman-3-yl- |
| | methyl)-1,2,3,4-tetrahydrobenzo[b][1,6]naphthyridine-10- |
| | carboxamide |
| “B49” | N-(1-Methylpiperidin-4-yl)-2-(6-chloro-2H-chromen-3- |
| | ylmethyl)-1,2,3,4-tetrahydrobenzo[b][1,6]naphthyridine- |
| | 10-carboxamide |
| “B50 | N-(1-Methylpiperidin-4-yl)-2-(6,8-dichloro-2-oxo-2H- |
| | chromene-3-carbonyl)-1,2,3,4-tetrahydrobenzo[b][1,6]- |
| | naphthyridine-10-carboxamide |
| “B51” | 1-(5-Chloro-2-hydroxyphenyl)-2-[10-(morpholine-4- |
| | carbonyl)-3,4-dihydro-1H-benzo[b][1,6]naphthyridin-2- |
| | yl]ethanone |
| “B52” | [2-(2-Chloropyridin-4-ylmethyl)-1,2,3,4-tetrahydro- |
| | benzo[b][1,6]naphthyridin-10-yl]morpholin-4-yl- |
| | methanone |
| “B53” | N-Piperidin-3-yl-2-[(5-chloro-2-methoxyphenyl- |
| | carbamoyl)methyl]-1,2,3,4-tetrahydrobenzo[b][1,6]- |
| | naphthyridine-10-carboxamide |
| “B54” | N-(1-Methylpiperidin-4-yl)-2-[2-(5-bromo-2- |
| | methoxyphenyl)thiazol-4-ylmethyl]-1,2,3,4-tetra- |
| | hydrobenzo[b][1,6]naphthyridine-10-carboxamide |
| “B55” | N-(1-Methylpiperidin-4-yl)-2-[2-(5-bromo-2-methoxy- |
| | phenyl)thiazol-4-ylmethyl]-1,2,3,4-tetrahydrobenzo- |
| | [b][1,6]naphthyridine-10-carboxamide |
| “B56” | {2-[2-(5-Chloro-2-methoxyphenyl)pyridin-4-ylmethyl]- |
| | 1,2,3,4-tetrahydrobenzo[b][1,6]naphthyridin-10-yl}- |
| | morpholin-4-ylmethanone |
| “B57” | N-(1-Methylpiperidin-4-yl)-2-(5,7-dichloro-8-hydroxy- |
| | quinolin-2-ylmethyl)-1,2,3,4-tetrahydrobenzo[b][1,6]- |
| | naphthyridine-10-carboxamide |

and pharmaceutically usable salts and stereoisomers thereof, including mixtures thereof in all ratios.

10. Process for the preparation of compounds of the formula I and pharmaceutically usable salts, and stereoisomers thereof, characterised in that a compound of the formula II 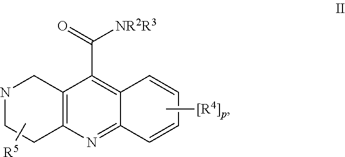
in which R², R³, R⁴, R⁵ and p, have the meanings indicated in claim 1,
is reacted with a compound of the formula III or formula IV 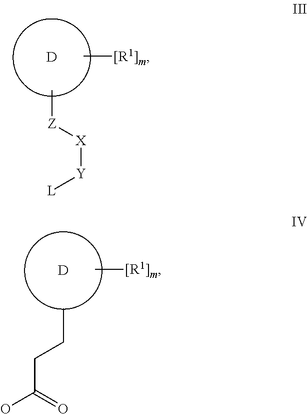
in which
R¹, m, D, Z, X and Y have the meanings indicated in claim 1 and L is a halogen, tosylate, mesylate or triflate,
and/or a base or acid of the formula I is converted into one of its salts.

11. Medicaments comprising at least one compound of the formula I according to claim 1 and/or pharmaceutically usable salts and stereoisomers thereof, including mixtures thereof in all ratios, and optionally excipients and/or adjuvants.

12. A method for the treatment of diseases in which the inhibition, regulation and/or modulation of phosphodiesterase or lysophospholipase autotaxin plays a role, comprising administering to a patient an effective amount of a compound according to claim 1.

13. A method for the treatment and prophylaxis of cancer diseases, comprising administering to a patient an effective amount of a compound according to claim 1.

14. A method according to claim 13, where the cancer diseases are associated with a tumour from the group of tumours of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx and/or of the lung.

15. A method according to claim 14, where the tumour originates from the group monocytic leukaemia, lung adenocarcinoma, small-cell lung carcinomas, pancreatic cancer, ovarian carcinoma, glioblastomas and breast carcinoma and colon carcinoma.

16. A method according to claim 15, where the disease to be treated is a tumour of the blood and immune system.

17. A method according to claim 16, where the tumour originates from the group of acute myeloid leukaemia, chronic myeloid leukaemia, acute lymphatic leukaemia and/or chronic lymphatic leukaemia.

18. A method for the treatment of tumours where a therapeutically effective amount of a compound according to claim 1 is administered in combination with radiotherapy and a compound from the group 1) oestrogen receptor modulator, 2) androgen receptor modulator, 3) retinoid receptor modulator, 4) cytotoxic agent, 5) antiproliferative agent, 6) prenyl-protein transferase inhibitor, 7) HMG-CoA reductase inhibitor, 8) HIV protease inhibitor, 9) reverse transcriptase inhibitor and 10) other angiogenesis inhibitors.
